(19) United States
(12) Patent Application Publication
(10) Pub. No.: US 2026/0245063 A1
(43) Pub. Date: Aug. 20, 2026

(54) APPARATUS AND METHOD FOR PROVIDING REAL-TIME PAYMENT TRANSACTIONS AND/OR PEER-TO-PEER PAYMENT TRANSACTIONS AND SECURITY FOR THE SAME

(71) Applicant: RAYMOND ANTHONY JOAO, YONKERS, NY (US)

(72) Inventor: RAYMOND ANTHONY JOAO, YONKERS, NY (US)

(21) Appl. No.: 19/444,332

(22) Filed: Jan. 9, 2026

Related U.S. Application Data
(60) Provisional application No. 63/760,684, filed on Feb. 20, 2025.

(51) Int. Cl. G06Q 20/22; G06Q 20/38; G16H 10/60

(52) U.S. Cl. CPC G06Q 20/223; G06Q 20/389; G16H 10/60

(57) ABSTRACT

An apparatus, including a computer, a distributed ledger system; and a payment system. The computer receives information regarding a requested payment from a counterparty device of a counterparty payee, verifies the counterparty payee account, and generates a payment request message. The computer transmits the payment request message to a user device of a user payor, and receives a payment request response message. The computer verifies the user payor account and instructs the payment system to execute a real-time payment or peer-to-peer payment, executes a real-time payment or peer-to-peer payment, or instructs the distributed ledger technology system to execute a real-time payment or peer-to-peer payment. The computer generates a message and transmits the message to the user device and counterparty device. The computer stores information regarding the real-time payment or peer-to-peer payment in a database, the distributed ledger technology system, or a database and the distributed ledger technology system.

### RELATED APPLICATIONS

This application claims the benefit of the priority of U.S. Provisional Patent Application Ser. No. 63/760,684, filed Feb. 20, 2025, and entitled “APPARATUS AND METHOD FOR PROVIDING REAL-TIME PAYMENT TRANSACTIONS AND/OR PEER-TO-PEER PAYMENT TRANSACTIONS AND SECURITY FOR THE SAME”, the subject matter and teachings of which are hereby incorporated by reference herein in their entirety.

### FIELD OF THE INVENTION

The present invention pertains to an apparatus and method for providing real-time payment transactions and/or peer-to-peer payment transactions and security for the same and, in particular, the present invention pertains to an apparatus and method for providing real-time payment transactions and/or peer-to-peer payment transactions and security for the same which provides security for ensuring that payments are effectuated in a secure manner.

### BACKGROUND OF THE INVENTION

There can be no doubt that payments and monetary transfers are part of everyday life, and that the use of various payment systems play a crucial role in our society and world. Various conventional payment systems include debit card payment systems, credit card payment systems, Automated Clearing House (ACH) payment systems, and wire transfer payment systems. These aforementioned payment systems have costs associated with the same and require time in their execution. While the aforementioned conventional payment systems have costs associated therewith and take time to effectuate funds availability to a respective payee, they do provide safeguards against mistaken transactions, payments to the incorrect parties, and/or fraud.

Real-time payments and peer-to-peer payment platforms and systems can effectuate payments in real-time (as for real-time payments) or can effectuate payments much quicker than the time needed in the aforementioned conventional payment systems (as for peer-to-peer payments) although not in real-time. Real-time payments and peer-to-peer payments, however, lack the safeguards associated with the aforementioned conventional payment systems. Although Real-time payments and peer-to-peer payments can be effectuated more quickly and provide for much cheaper transaction fees than the aforementioned conventional payment systems, they are typically irreversible in the cases of mistaken transactions, payments made to incorrect parties, and/or fraud.

The use of the distributed ledger technologies, which can include blockchain technologies and/or other substitute technologies can also be utilized in real-time payment platforms, peer-to-peer payment platforms, and/or other payment platforms. These distributed ledger payment platforms share the same shortcomings as in real-time payment platforms, peer-to-peer payment platforms as they are typically irreversible in the cases of mistaken transactions, payments made to incorrect parties, and/or fraud.

In view of the foregoing, there is a need for technological improvements in payment systems which address and overcome the shortcomings of the above-described prior art systems.

### SUMMARY OF THE INVENTION

The present invention pertains to an apparatus and method for providing real-time payment transactions and/or peer-to-peer payment transactions and security for the same which overcomes the shortfalls of the prior art. The present invention pertains to an apparatus and method for providing real-time payment transactions and/or peer-to-peer payment transactions and security for the same. In this regard, the present invention can be utilized to provide for secured real-time payments and/or secured peer-to-peer payments notwithstanding the fact that such payments are typically irreversible.

The present invention includes a central processing computer/distributed ledger technology system, which can include a central processing computer and a distributed ledger technology system. The central processing computer/distributed ledger technology system, the central processing computer, and/or the distributed ledger technology system by itself, can perform any of the processing routines and functionality typically performed by any transaction processing computer used in processing and/or facilitating real-time payments and/or peer-to-peer payments.

The central processing computer/distributed ledger technology system and/or the distributed ledger technology system by itself, can perform any of the processing routines and functionality typically performed by any transaction processing computer used in processing and/or facilitating real-time payments and/or peer-to-peer payments, and/or cryptocurrency payments involving a blockchain technology system, or a linear blockchain data structure, or a directed acyclic graph (DAG) technology system, and/or any combination of the same.

The central processing computer/distributed ledger technology system, and/or the central processing computer of the same, and/or the distributed ledger technology system of the same, can also be utilized to process any transaction or transactions involving, on, or regarding, credit card accounts, credit accounts, debit card accounts, debit accounts, charge card accounts, charge accounts, bank accounts, checking accounts, savings accounts, electronic money accounts, gambling accounts, gaming accounts, sports betting accounts, lottery gaming accounts, brokerage accounts, healthcare insurance accounts and/or healthcare payer or payor accounts, cryptocurrency accounts, cryptocurrencies, and/or any of other account(s) described herein or otherwise. The central processing computer/distributed ledger technology system can also perform any the processing routines and/or functionality described herein as being performed by the present invention.

Any number of central processing computers can be utilized in connection in or with the central processing computer/distributed ledger technology system and/or in or with the present invention. The distributed ledger technology system can include and/or can utilize a blockchain technology system and/or any number of blockchain technology systems, and/or the distributed ledger technology system can include and/or can utilize a directed acyclic graph (DAG) technology system and/or any number of directed acyclic graph (DAG) technology systems, and/or any combination of the above.

The present invention also includes a user communication device which can be utilized by any individual or account holder who or which utilizes the present invention. The user communication device can be utilized to communicate with, transmit signals, data, information, or a message, to, receive signals, data, information, or a message, from, or to access, or which can be linked with, or which can be wirelessly linked with, any of the central processing computer/distributed ledger technology system, and/or any central processing computer or distributed ledger technology system of the same.

The present invention can also include a user wallet which can be a component of, and/or integrated within, the user communication device, or the user wallet can be located separate and apart from and/or located externally from the user communication device. The user wallet can store any data and/or information and/or payment account data and/or information needed, desired, to required, for making or effectuating real-time payments, peer-to-peer payments, and/or cryptocurrency payments. The user wallet can be any suitable and/or commercially available wallet or wallet software or software application.

The user communication device can be connected to or with, and/or linked to or with, the user wallet, and/or the user wallet can be a component of, and/or integrated within, the user communication device. The user wallet can be connected to, linked with, and/or wirelessly connected to or linked with, the central processing computer/distributed ledger technology system, and/or any central processing computer or distributed ledger technology system of the same, and/or the user wallet can be connected to, linked with, and/or wirelessly connected to or linked with, the user communication device. The user wallet can store account information, can store funds or cryptocurrencies of and/or for the user, individual, or account holder, and/or can store any data and/or information regarding any accounts, funds, or cryptocurrencies, of or for the user, individual, or account holder.

The present invention can also include a user bank/financial institution records computer. The user bank/financial institution records computer can be a, or any, transaction processing computer(s) of, for, or associated with, any bank, thrift, credit union, financial intermediary, or cryptocurrency exchange or wallet, which is used for storing funds or cryptocurrency or cryptocurrencies of or for the user, individual, or account holder. The user bank/financial institution records computer can be connected to, or linked with the central processing computer/distributed ledger technology system, and/or any central processing computer or distributed ledger technology system of the same. The user bank/financial institution records computer can be connected to, or linked with, the user wallet and/or the user communication device, either via a wired connection, a wireless connection, and/or any combination of the same.

The present invention can also include a counterparty communication device which can be utilized by any counterparty who or which is involved in a transaction with any individual or account holder who or which utilizes the present invention. The counterparty communication device can be utilized to communicate with, transmit signals, data, information, or a message, to, receive signals, data, information, or a message, from, or to access, or which can be linked with, or which can be wirelessly linked with, any of the central processing computer/distributed ledger technology system, and/or any central processing computer or distributed ledger technology system of the same.

The present invention can also include a counterparty wallet which can be a component of, and/or integrated within, the counterparty communication device, or the counterparty wallet can be located separate and apart from and/or located externally from the counterparty communication device. The counterparty wallet can store any data and/or information and/or payment account data and/or information needed, desired, to required, for making or effectuating real-time payments, peer-to-peer payments, and/or cryptocurrency payments. The counterparty wallet can be any suitable and/or commercially available wallet or wallet software or software application.

The counterparty communication device can be connected to or with, and/or linked to or with, the counterparty wallet, and/or the counterparty wallet can be a component of, and/or integrated within, the counterparty communication device. The counterparty wallet can be connected to, linked with, and/or wirelessly connected to or linked with, the central processing computer/distributed ledger technology system, and/or any central processing computer or distributed ledger technology system of the same, and/or the counterparty wallet can be connected to, linked with, and/or wirelessly connected to or linked with, the counterparty communication device.

The counterparty wallet can store account information, can store funds or cryptocurrencies of and/or for the counterparty, and/or can store any data and/or information regarding any accounts, funds, or cryptocurrencies, of or for the counterparty.

The present invention can also include a counterparty bank/financial records computer. The counterparty bank/financial records computer can be a, or any, transaction processing computer(s) of, for, or associated with, any bank, thrift, credit union, financial intermediary, or cryptocurrency exchange or wallet, which is used for storing funds or cryptocurrency or cryptocurrencies of or for the counterparty. The counterparty bank/financial records computer can be connected to, or linked with the central processing computer/distributed ledger technology system, and/or any central processing computer or distributed ledger technology system of the same. The counterparty bank/financial records computer can be connected to, or linked with, the counterparty wallet and/or the counterparty communication device, either via a wired connection, a wireless connection, and/or any combination of the same.

The user communication device and/or the user wallet can be connected to, or linked with, the user bank/financial institution records computer so as to allow the user communication device and/or the user wallet to communicate with the user bank/financial institution records computer in a bi-directional manner. The counterparty communication device and/or the counterparty wallet can be connected to, or linked with, the counterparty bank/financial institution records computer so as to allow the counterparty communication device and/or the counterparty wallet to communicate with the counterparty bank/financial institution records computer in a bi-directional manner.

The present invention can also include a real-time/peer-to-peer payment provider system, which can be any commercially available real-time or peer-to-peer payment system of platform which can be operated by private entities, by banks, by central banks, or by any other entities. The real-time/peer-to-peer payment provider system can be operated by any financial services company, any bank, any financial institution, any financial intermediary, any brokerage firm, any financial services company or entity, any entity formed by one or more U.S. financial institutions and/or foreign financial institutions, the U.S. Federal Reserve System or a Federal Reserve Bank, and/or any other central bank of any country or territory.

The central processing computer/distributed ledger technology system and/or the central processing computer or distributed ledger technology system of the same, can be connected or linked to or with, or wirelessly linked to or with, the real-time/peer-to-peer payment provider system. The central processing computer/distributed ledger technology system, and/or any central processing computer or distributed ledger technology system of the same, the user communication device, the user wallet, the user bank/financial institution records computer, the counterparty communication device, the counterparty wallet, the counterparty bank/financial records computer, can communicate with, can communicate with, can transmit signals, messages, data, and/or information to, and/or can receive signals, messages, data, and/or information from, the real-time/peer-to-peer payment provider system, in a bi-directional manner.

The present invention can be utilized in effectuating any type of real-time payment of peer-to-peer payment involving or in including any type or kind of currency, fiat currency, cryptocurrency, or any other medium of exchange.

The central processing computer can include a central processing unit or CPU, a random access memory device(s) (RAM), a read only memory device(s) (ROM), a user input device, a display device, a transmitter, a receiver, a database, an output device, a global positioning device, and/or a video and/or audio recording device.

The user communication device can also include a central processing unit or CPU, a random access memory device(s) (RAM), a read only memory device(s) (ROM), a user input device, a display device, a transmitter, a receiver, a database, an output device, a global positioning device, and/or a video and/or audio recording device.

The counterparty communication device can also include a central processing unit or CPU, a random access memory device(s) (RAM), a read only memory device(s) (ROM), a user input device, a display device, a transmitter, a receiver, a database, an output device, a global positioning device, and/or a video and/or audio recording device.

The present invention can be utilized to in order to provide and/or to process real-time payments and/or real-time payment transactions and/or peer-to-peer payments and/or peer-to-peer payment transactions, and to provide for the security of the same. The present invention can be utilized to in order to provide and/or to process real-time payments and/or real-time payment transactions and/or peer-to-peer payments and/or peer-to-peer payment transactions, and to provide for the security of the same in transactions involving any two or more individuals and/or entities.

The present invention can also be utilized to in order to provide and/or to process real-time payments and/or real-time payment transactions and/or peer-to-peer payments and/or peer-to-peer payment transactions, and to provide for the security of the same in transactions involving any number or types of accounts serviced by any bank, financial institution, or financial intermediary, or for any number or types of accounts serviced by any number of banks, financial institutions, or financial intermediaries.

The present invention can also be utilized to in order to provide and/or to process real-time payments and/or real-time payment transactions and/or peer-to-peer payments and/or peer-to-peer payment transactions, and provide for the security of the same in transactions involving any number, or any type or kind, of an any, or any combination of, credit accounts, credit card accounts, debit accounts, debit card accounts, charge accounts, charge card accounts, bank accounts, checking accounts, savings accounts, electronic money accounts, cryptocurrency accounts and/or cryptocurrencies, gambling accounts, gaming accounts, sports betting accounts, lottery gaming accounts, brokerage accounts, healthcare insurance accounts, and/or any of the other accounts described herein, such as, but not limited to, pension accounts, individual retirement accounts (IRAs), or self-employed pension (SEP) accounts, “smart” card accounts, currency card accounts, healthcare accounts, Medicare accounts, Medicaid accounts, employee benefits accounts, cafeteria accounts, or spending accounts, subscription accounts for any goods, products, or services, or insurance accounts, healthcare spending accounts, life insurance accounts, or disability insurance accounts, or tuition accounts, pharmacy accounts, credit report accounts, cable television accounts, digital television accounts, or satellite television accounts, social security accounts, liability insurance accounts, or lease insurance accounts, ticket accounts, telephone calling card accounts, utility accounts, electrical utility accounts, gas utility accounts, or fuel oil utility accounts, accounts monitoring use of official seals, accounts monitoring use of private, individual, and/or organizational, seals or access codes, security access accounts, computer access code accounts, facility access accounts, or facility security accounts, financial accounts, or electronic cash accounts, communication accounts, telephone accounts, wireless communication device accounts, non-wireless communication device accounts, cellular communication device accounts, cellular telephone accounts, Internet accounts, or Internet service provider accounts, electronic signature accounts, e-mail accounts, membership accounts, club membership accounts, entertainment membership accounts, entertainment tickets accounts, sports tickets accounts, theatre tickets accounts, concert or opera tickets accounts, consumer or purchaser memberships accounts, sports club membership accounts, or health club membership accounts, merchant credit accounts for customers, merchant accounts, association membership accounts, professional association membership accounts, or trade association membership accounts, or any other accounts described herein, as well as any cards, devices, and/or other entities, which can be used with or which can be associated with any of the herein-described accounts.

The present invention can also be utilized to in order to provide and/or to process real-time payments and/or real-time payment transactions and/or peer-to-peer payments and/or peer-to-peer payment transactions, in and pursuant to healthcare claims payments to healthcare providers and/or other providers. The present invention can also be a component of, or can be integrated with or within, any apparatus or platform, including any and/or all of those which are disclosed in all of the herein-identified patents and/or patent applications which are incorporated by reference into this application as well as any healthcare platforms, electronic healthcare records and/or any other records and/or telehealth platforms or systems the teachings of which are hereby incorporated by reference herein.

The present invention can be used to effectuate a real-time payment or a peer-to-peer payment from a user to a counterparty while providing for the verification of the parties involved and their respective wallets and/or their respective bank account or financial account or cryptocurrency account.

The present invention can also be utilized so as to ensure, and to verify, before any payment is initiated from a payor's (or user's) wallet or a payor's (or user's) account, that the payment will be effectuated or made to a correct wallet or account of a payee (or counterparty). The present invention can utilize data and/or information regarding a position or location of the payor (or user) and/or a payee (or counterparty) and/or the respective communication devices of the payor (or user) or the payee (or counterparty) during a transaction.

The present invention can also be utilized to provide an escrow feature, which can be effectuated and/or provided by and/or with the central processing computer/distributed ledger technology system, and/or by or with the central processing computer and/or the distributed ledger technology system, so as to provide for funds security for and/or during a real-time payment transaction or funds transfer transaction and/or a peer-to-peer payment transaction or funds transfer transaction.

The present invention can also utilize one or any number of smart contracts in order to provide an escrow feature so as to provide for funds security for and/or during a real-time payment transaction or funds transfer transaction and/or a peer-to-peer payment transaction or funds transfer transaction.

The present invention can also be utilized in and/or as a component of, an electronic healthcare record (EHR) system or platform in order to process healthcare insurance payments or healthcare payor payments to a healthcare provider upon the providing of healthcare services or healthcare-related services to an individual or patient.

The present invention can also be utilized in connection with the real-time/peer-to-peer payment provider system or with any one or more, or any number of, real-time/peer-to-peer payment provider system(s), in order to effectuate a real-time payment or a peer-to-peer payment. The central processing computer/distributed ledger technology system, and/or the central processing computer and/or the distributed ledger technology system, can also be utilized in order to effectuate a real-time payment or a peer-to-peer payment.

The present invention can be utilized in order to process and/or effectuate a real-time payment and/or a peer-to-peer payment between a user and a counterparty in order to ensure that the payment of funds is securely made or effectuated made from the account of the user (the payor) to the account of the counterparty (the payee).

The present invention can also be utilized to process and to effectuate real-time payments and/or peer-to-peer payments involving payments requested by healthcare providers from healthcare insurers and/or healthcare payer pursuant to previously agreed upon payment allowances and/or payment schedules.

The present invention can also be integrated into an electronic healthcare record (EHR), and/or any an electronic medical record, an electronic dental record, an electronic pharmacy record, an electronic behavioral health record, or a personal healthcare record, which can, in addition to generating insurance claims forms or messages or request for payments forms or messages, can also provided for the real-time payments and/or the peer-to-peer payments of and for the same.

A smart contract or any number of smart contracts, utilized in the distributed ledger technology system can be utilized to perform any and/or all of the processing steps described herein as being performed by the central processing computer

The present invention also be utilized and can work in conjunction with, can be integrated into or with, and/or can perform all of the functions and functionalities disclosed and described as being performed by each of, the respective apparatuses and their various and respective central processing computers, computers, and/or communication devices, which are disclosed and described in the U.S. Patent Applications and U.S. Provisional Patent Applications which are also incorporated by reference herein.

The present invention can also be utilized by healthcare providers to request automatic payment upon automated approval of a healthcare insurance claim or a claim for payment from a healthcare insurer of a healthcare payer.

The central processing computer, and/or a smart contract or any number of smart contracts utilized in the distributed ledger technology system, can also process IP address information or position or location information for a respective user communication device and/or a counterparty communication device in or order to verify an identify and/or an authority of a respective user payor and/or a counterparty payee.

The central processing computer, and/or a smart contract or any number of smart contracts utilized in the distributed ledger technology system, can also process information obtained from a fingerprint recognition device, a voice recognition device, a retinal scanner, a fingerprint device, a thumbprint device, a voice recognition device, a handprint recognition device, a handprint geometry recognition device, a facial feature recognition device, and/or any one or more of the biometric devices, in or order to verify an identify and/or an authority of a respective user payor and/or a counterparty payee.

The central processing computer, and/or a smart contract or any number of smart contracts utilized in the distributed ledger technology system, can also process information for verifying a bank account, financial account, or other account or cryptocurrency, and/or respective user wallet or counterparty wallet in or order to verify an identify or authority of a respective user payor and/or a counterparty payee.

The central processing computer, and/or a smart contract or any number of smart contracts utilized in the distributed ledger technology system, can also process information regarding an limitation(s) and/or restriction(s) placed by a user payor on or involving any bank account, financial account, and/or other account, and/or any cryptocurrency, in order to determine if a real-time payment or a peer-to-peer payment is allowed or authorized.

The central processing computer can also process information for instructing, effectuating, and/or providing notification to a user payor and/or a counterparty payee, a real-time payment or a peer-to-peer payment.

The central processing computer can also process information for instructing, effectuating, and/or providing notification to a user payor and/or a counterparty payee, a real-time payment or a peer-to-peer payment.

A smart contract or any number of smart contracts utilized in the distributed ledger technology system can process information for instructing, effectuating, and/or providing notification to a user payor and/or a counterparty payee, a real-time payment or a peer-to-peer payment.

A smart contract or any number of smart contracts utilized in the distributed ledger technology system can process information for instructing, effectuating, and/or providing notification to a user payor and/or a counterparty payee, a real-time payment or a peer-to-peer payment.

The present invention can be utilized in conjunction with, and/or can be integrated with or within, any apparatus, system, or platform, which can involve any processing of any real-time payment or peer-to-peer payment.

The present invention provides for substantial technological improvements to and for real-time payment systems computers and/or platforms, and/or peer-to-peer payment systems computers and/or platforms. The present invention also provides for substantial technological improvements to and for electronic healthcare records computers, systems, and/or platforms and/or telehealth computers, systems, and/or platforms, which can provide for the processing of real-time payments and/or peer-to-peer payments, via the same.

The present invention provides for substantial technological improvements to and for real-time payment systems computers and/or platforms, and/or peer-to-peer payment systems computers and/or platforms which can provide enhanced security for and involving real-time payments and/or peer-to-peer payments. The present invention also provides for substantial technological improvements to and for electronic healthcare records computers, systems, and/or platforms and/or telehealth computers, systems, and/or platforms, which can provide enhanced security for and involving real-time payments and/or peer-to-peer payments.

### BRIEF DESCRIPTION OF THE DRAWINGS

In the Drawings:

FIG. 1 illustrates a preferred embodiment of the apparatus of the present invention, in block diagram form;

FIG. 2 illustrates a preferred embodiment of the central processing computer/distributed ledger technology system of FIG. 1, in block diagram form;

FIG. 3 illustrates a preferred embodiment of the user communication device of FIG. 1, in block diagram form;

FIG. 4 illustrates a preferred embodiment of the counterparty communication device of FIG. 1, in block diagram form; and

FIGS. 5A, 5B, and 5C illustrate a preferred embodiment method for utilizing the apparatus of the present invention, in flow diagram form; and

FIGS. 6A and 6B illustrate another preferred embodiment method for utilizing the apparatus and method of the present invention, in block diagram form.

### DESCRIPTION OF THE PREFERRED EMBODIMENTS

The present invention pertains to an apparatus and method for providing real-time payment transactions and/or peer-to-peer payment transactions and security for the same. In this regard, the present invention can be utilized to provide for secured real-time payments and/or secured peer-to-peer payments notwithstanding the fact that such payments are typically irreversible.

Applicant hereby incorporates by reference herein the subject matter and teachings of U.S. Provisional Patent Application Ser. No. 63/760,684, filed Feb. 20, 2025, and entitled “APPARATUS AND METHOD FOR PROVIDING REAL-TIME PAYMENT TRANSACTIONS AND/OR PEER-TO-PEER PAYMENT TRANSACTIONS AND SECURITY FOR THE SAME”, the subject matter and teachings of which are hereby incorporated by reference herein in their entirety.

Applicant hereby incorporates by reference herein the subject matter and teachings of U.S. patent application Ser. No. 16/509,026, filed Jul. 14, 2019, and entitled “APPARATUS AND METHOD FOR PROVIDING TRANSACTION SECURITY AND/OR ACCOUNT SECURITY”, the subject matter and teachings of which are hereby incorporated by reference herein in their entirety.

Applicant hereby incorporates by reference herein the subject matter and teachings of U.S. patent application Ser. No. 16/029,550, filed Jul. 7, 2018, and entitled “APPARATUS AND METHOD FOR PROVIDING TRANSACTION SECURITY AND/OR ACCOUNT SECURITY”, the subject matter and teachings of which are hereby incorporated by reference herein in their entirety.

Applicant hereby incorporates by reference herein the subject matter and teachings of U.S. Provisional Patent Application Ser. No. 62/716,779, filed Aug. 9, 2018, and entitled “APPARATUS AND METHOD FOR PROVIDING TRANSACTION SECURITY AND/OR ACCOUNT SECURITY”, the subject matter and teachings of which are hereby incorporated by reference herein in their entirety.

Applicant also hereby incorporates by reference herein the subject matter and teachings of U.S. Provisional Patent Application Ser. No. 62/715,110, filed Aug. 6, 2018, and entitled “APPARATUS AND METHOD FOR PROVIDING TRANSACTION SECURITY AND/OR ACCOUNT SECURITY”, the subject matter and teachings of which are hereby incorporated by reference herein in their entirety.

Applicant also hereby incorporates by reference herein the subject matter and teachings of each of U.S. patent application Ser. No. 14/458,316, filed Aug. 13, 2014, and entitled “APPARATUS AND METHOD FOR PROVIDING TRANSACTION SECURITY AND/OR ACCOUNT SECURITY”, U.S. patent application Ser. No. 14/328,434, filed Jul. 10, 2014, and entitled “APPARATUS AND METHOD FOR PROVIDING TRANSACTION SECURITY AND/OR ACCOUNT SECURITY”, U.S. patent application Ser. No. 14/283,820, filed May 21, 2014, and entitled “APPARATUS AND METHOD FOR PROVIDING TRANSACTION SECURITY AND/OR ACCOUNT SECURITY”, U.S. patent application Ser. No. 14/289,673, filed May 29, 2014, and entitled “APPARATUS AND METHOD FOR PROVIDING TRANSACTION SECURITY AND/OR ACCOUNT SECURITY”, U.S. Provisional Patent Application Ser. No. 62/035,541, filed Aug. 11, 2014, and entitled “APPARATUS AND METHOD FOR PROVIDING TRANSACTION SECURITY AND/OR ACCOUNT SECURITY”, U.S. Provisional Patent Application Ser. No. 61/880,757, filed Sep. 20, 2013, and entitled “APPARATUS AND METHOD FOR PROVIDING TRANSACTION SECURITY AND/OR ACCOUNT SECURITY”, U.S. Provisional Patent Application Ser. No. 61/957,360, filed Jul. 1, 2013, and entitled “APPARATUS AND METHOD FOR PROVIDING TRANSACTION SECURITY AND/OR ACCOUNT SECURITY”, and U.S. Provisional Patent Application Ser. No. 61/956,801, filed Jun. 17, 2013, and entitled “APPARATUS AND METHOD FOR PROVIDING TRANSACTION SECURITY AND/OR ACCOUNT SECURITY”, the subject matter and teachings of all of which herein-identified applications are hereby incorporated by reference herein in their entirety.

Applicant also hereby incorporates by reference herein the subject matter and teachings of each of U.S. Provisional Patent Application Ser. No. 63/634,841, filed Apr. 16, 2024, and entitled “APPARATUS AND METHOD FOR PROVIDING HEALTHCARE SERVICES REMOTELY OR VIRTUALLY WITH OR USING AN ELECTRONIC HEALTHCARE RECORD AND/OR A COMMUNICATION NETWORK AND/OR WITH OR USING A PERSONAL MONITORING DEVICE”, filed April 2024, and U.S. Provisional Patent Application Ser. No. 63/643,488, filed May 7, 2025, and entitled “APPARATUS AND METHOD FOR PROVIDING HEALTHCARE SERVICES REMOTELY OR VIRTUALLY WITH OR USING AN ELECTRONIC HEALTHCARE RECORD AND/OR A COMMUNICATION NETWORK AND/OR WITH OR USING A PERSONAL MONITORING DEVICE”, the subject matter and teachings of all of which herein-identified applications are hereby incorporated by reference herein in their entirety.

Applicant also hereby incorporates by reference herein the subject matter and teachings of U.S. patent application Ser. No. 17/224,838, filed Apr. 7, 2021, and entitled “APPARATUS AND METHOD FOR PROVIDING HEALTHCARE SERVICES REMOTELY OR VIRTUALLY WITH OR USING AN ELECTRONIC HEALTHCARE RECORD AND/OR A COMMUNICATION NETWORK”, the subject matter and teachings of which are hereby incorporated by reference herein in their entirety.

Applicant also hereby incorporates by reference herein the subject matter and teachings of U.S. Provisional Patent Application Ser. No. 63/634,841, filed Apr. 16, 2024, and entitled “APPARATUS AND METHOD FOR PROVIDING HEALTHCARE SERVICES REMOTELY OR VIRTUALLY WITH OR USING AN ELECTRONIC HEALTHCARE RECORD AND/OR A COMMUNICATION NETWORK AND/OR WITH OR USING A PERSONAL MONITORING DEVICE”, filed April 2024, and U.S. Provisional Patent Application Ser. No. 63/643,488, filed May 7, 2025, and entitled “APPARATUS AND METHOD FOR PROVIDING HEALTHCARE SERVICES REMOTELY OR VIRTUALLY WITH OR USING AN ELECTRONIC HEALTHCARE RECORD AND/OR A COMMUNICATION NETWORK AND/OR WITH OR USING A PERSONAL MONITORING DEVICE”, the subject matter and teachings of all of which are hereby incorporated by reference herein in their entirety.

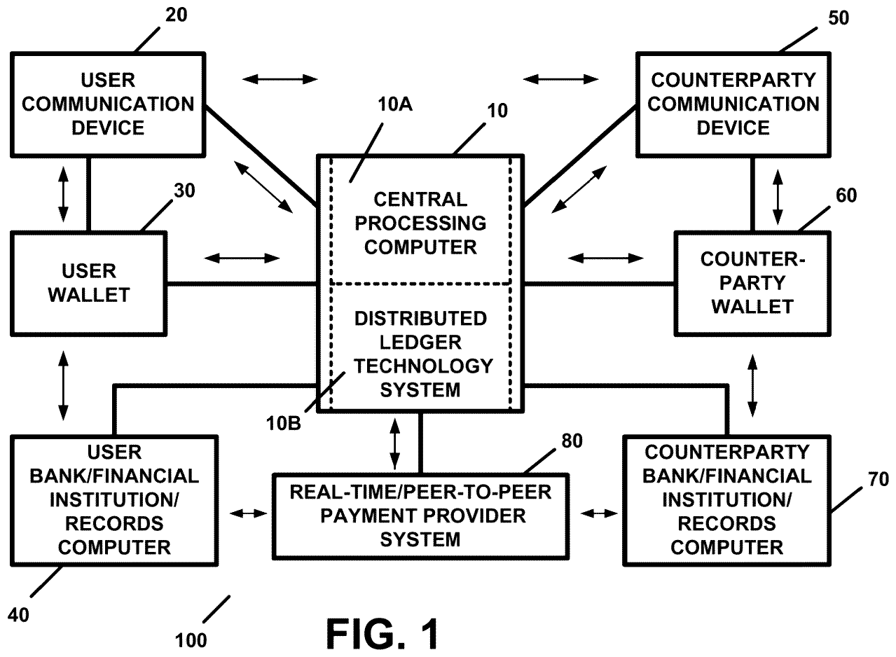

FIG. 1 illustrates a preferred embodiment of the apparatus of the present invention which is designated by the reference numeral 100, in block diagram form.

With reference to FIG. 1, the apparatus 100 includes a central processing computer/distributed ledger technology system 10. In a preferred embodiment, the central processing computer/distributed ledger technology system 10 includes a central processing computer 10A and a distributed ledger technology system 10B. In a preferred embodiment, central processing computer/distributed ledger technology system 10, the central processing computer 10A by itself, and/or the distributed ledger technology system 10B by itself, can perform any of the processing routines and functionality typically performed by any transaction processing computer used in processing and/or facilitating real-time payments and/or peer-to-peer payments.

In a preferred embodiment, the central processing computer/distributed ledger technology system 10 and/or the distributed ledger technology system 10B by itself, can perform any of the processing routines and functionality typically performed by any transaction processing computer used in processing and/or facilitating real-time payments and/or peer-to-peer payments, and/or cryptocurrency payments involving a blockchain technology system, or a linear blockchain data structure, or a directed acyclic graph (DAG) technology system, and/or any combination of the same.

To the extent needed or required, in a preferred embodiment, the central processing computer/distributed ledger technology system 10, and/or the central processing computer 10A of the same, and/or the distributed ledger technology system 10B of the same, can also be utilized to process any transaction or transactions involving, on, or regarding, credit card accounts, credit accounts, debit card accounts, debit accounts, charge card accounts, charge accounts, bank accounts, checking accounts, savings accounts, electronic money accounts, gambling accounts, gaming accounts, sports betting accounts, lottery gaming accounts, brokerage accounts, healthcare insurance accounts and/or healthcare payer or payor accounts, cryptocurrency accounts, cryptocurrencies, and/or any of other account(s) described herein or otherwise. In a preferred embodiment, the central processing computer/distributed ledger technology system 10 can also perform any the processing routines and/or functionality described herein as being performed by the apparatus 100 of the present invention.

In a preferred embodiment, any number of central processing computers 10A can be utilized in connection in or with the central processing computer/distributed ledger technology system 10 and/or in or with the apparatus 100. In the preferred embodiment, any central processing computer 10A can be dedicated to performing transaction authorization processing for a given type of account or any number of types of accounts. In a preferred embodiment, any number of distributed ledger technology system 10B can be utilized in connection in or with the central processing computer/distributed ledger technology system 10 and/or in or with the apparatus 100. In the preferred embodiment, any distributed ledger technology system 10B can be dedicated to performing transaction authorization processing for a given type of account or any number of types of accounts.

In a preferred embodiment, the distributed ledger technology system 10B can include and/or can utilize a blockchain technology system and/or any number of blockchain technology systems, and/or the distributed ledger technology system 10B can include and/or can utilize a directed acyclic graph (DAG) technology system and/or any number of directed acyclic graph (DAG) technology systems, and/or any combination of the above.

With reference once again to FIG. 1, the apparatus 100 also includes a user communication device 20 (also referred to as “user device 20” or “communication device 20”) which can be utilized by any individual or account holder who or which utilizes the apparatus 100 of the present invention. In a preferred embodiment, the user communication device 20 can be utilized to communicate with, transmit signals, data, information, or a message, to, receive signals, data, information, or a message, from, or to access, or which can be linked with, or which can be wirelessly linked with, any of the central processing computer/distributed ledger technology system 10, and/or any central processing computer 10A or distributed ledger technology system 10B of the same.

In a preferred embodiment, the user communication device 20 can be connected with, linked to, wirelessly connected with, or wirelessly linked to or with, the central processing computer/distributed ledger technology system 10. In a preferred embodiment, the user communication device 20 can also be connected with, linked to, wirelessly connected with, or wirelessly linked to or with, the central processing computer/distributed ledger technology system 10 during operation of the apparatus 100 of the present invention as described herein, and/or at any desired time or times.

In a preferred embodiment, the user communication device 20 can be a personal computer, a laptop computer, a notebook computer, a tablet, a tablet computer, a cellular telephone, a personal digital assistant, a wireless telephone, a wireless communication device, a personal communication device, a personal communications services device, a smart phone, a Smartphone, a mobile telephone, a hand-held device or computer, a palm-top device or computer, a watch, a telephone, a television, an interactive television, a digital television, a smart television or entertainment device, an internet-enabled television or entertainment device, or any other suitable device, which can be equipped to perform the functions described herein as being performed by the user communication device 20.

In a preferred embodiment, the user communication device 20 can include a central processing unit or device, an input device, a card reader, a barcode reader, a barcode scanner, a two-dimensional barcode reader, a two-dimensional barcode scanner, a QR code reader, a QR code scanner, an imaging device, a camera for obtaining an image of a barcode, a two-dimensional barcode, and/or a QR code, a two-dimensional image sensor, an account information data entry device, a card swiping device, a retinal scanning device, a fingerprint recognition device, a voice recognition device, a retinal scanner, a fingerprint device, a thumbprint device, a voice recognition device, a handprint recognition device, a handprint geometry recognition device, a facial feature recognition device, and/or any one or more of the biometric devices used to control access to a computer or a computer network which are known to those skilled in the art at the time of the filing of this patent application, a pointing device, a mouse, an output device, a database or a memory device and/or system, a random access memory (RAM) device, a read only memory (ROM) device, a video recording system or equipment, a camera(s), an audio recording system, device, or equipment, a microphone, a receiver or any number of receivers, a transmitter or any number of transmitters, a network interface device, an information or content gathering device, and/or any other devices, equipments, or systems, typically found in and/or utilized by any of the herein-described user communication devices 20 described herein as being utilized in connection with the apparatus 100 of the present invention. In a preferred embodiment, the user communication device 20 can also be equipped with a global positioning device which can be utilized to calculate, determine, or ascertain, the position or location of the user communication device 20.

In a preferred embodiment, the user communication device 20 can also contain, include, or be equipped with, a transmitter(s), a receiver(s), or any other network interface devices or equipment for facilitating bi-directional communication with, and/or data and/or information exchange with, the central processing computer/distributed ledger technology system 10.

In a preferred embodiment, the user communication device 20 can also include, contain, or be equipped with a camera, a digital video recording system or equipment, a microphone, a digital audio recording system or equipment, or any another digital video and audio recording device or equipment or other digital media recoding equipment, that can allow the user communication device 20 to record and store, for later play-back, any of the video and/or audio information which can or may be obtained using the apparatus 100 of the present invention. The user communication device 20 can also be used to take or record a photograph, picture, video, a video clip, audio, or an audio clip, of the account holder or any other user, individual, or entity.

In a preferred embodiment, the user communication device 20 can communicate, in bi-directional manner, with the central processing computer/distributed ledger technology system 10 and/or with any central processing computer 10A of the same and/or with any distributed ledger technology system 10B of the same.

In a preferred embodiment, any number of communication devices 20 can be assigned to, utilized by, or associated with, any individual or account holder.

In a preferred embodiment, the apparatus 100 also includes a user wallet 30. In a preferred embodiment, the user wallet 30 can be a component of, and/or integrated within, the user communication device 20 or the user wallet 30 can be located separate and apart from and/or located externally from the user communication device 20. In a preferred embodiment, the user wallet 30 can store any data and/or information and/or payment account data and/or information needed, desired, to required, for making or effectuating real-time payments, peer-to-peer payments, and/or cryptocurrency payments. In a preferred embodiment, the user wallet 30 can be any suitable and/or commercially available wallet or wallet software or software application.

In a preferred embodiment, the user communication device 20 can be connected to or with, and/or linked to or with, the user wallet 30, and/or the user wallet 30 can be a component of, and/or integrated within, the user communication device 20. In a preferred embodiment, the user wallet 30 can be connected to, linked with, and/or wirelessly connected to or linked with, the central processing computer/distributed ledger technology system 10, and/or any central processing computer 10A or distributed ledger technology system 10B of the same, and/or the user wallet 30 can be connected to, linked with, and/or wirelessly connected to or linked with, the user communication device 20.

In a preferred embodiment, the user wallet 30 can store account information, can store funds or cryptocurrencies of and/or for the user, individual, or account holder, and/or can store any data and/or information regarding any accounts, funds, or cryptocurrencies, of or for the user, individual, or account holder.

In a preferred embodiment, the apparatus 100 also includes a user bank/financial institution records computer 40. In a preferred embodiment, the user bank/financial institution records computer 40 can be a, or any, transaction processing computer(s) of, for, or associated with, any bank, thrift, credit union, financial intermediary, or cryptocurrency exchange or wallet, which is used for storing funds or cryptocurrency or cryptocurrencies of or for the user, individual, or account holder. In a preferred embodiment, the user bank/financial institution records computer 40 can be connected to, or linked with the central processing computer/distributed ledger technology system 10, and/or any central processing computer 10A or distributed ledger technology system 10B of the same. In another preferred embodiment, the user bank/financial institution records computer 40 can be connected to, or linked with, the user wallet 30 and/or the user communication device 20, either via a wired connection, a wireless connection, and/or any combination of the same.

With reference once again to FIG. 1, the apparatus 100 also includes a counterparty communication device 50 which can be utilized by any counterparty who or which is involved in a transaction with any individual or account holder who or which utilizes the apparatus 100 of the present invention. In a preferred embodiment, the counterparty communication device 50 can be utilized to communicate with, transmit signals, data, information, or a message, to, receive signals, data, information, or a message, from, or to access, or which can be linked with, or which can be wirelessly linked with, any of the central processing computer/distributed ledger technology system 10, and/or any central processing computer 10A or distributed ledger technology system 10B of the same.

In a preferred embodiment, the counterparty communication device 50 can be connected with, linked to, wirelessly connected with, or wirelessly linked to or with, the central processing computer/distributed ledger technology system 10. In a preferred embodiment, the counterparty communication device 50 can also be connected with, linked to, wirelessly connected with, or wirelessly linked to or with, the central processing computer/distributed ledger technology system 10 during operation of the apparatus 100 of the present invention as described herein, and/or at any desired time or times.

In a preferred embodiment, the counterparty communication device 50 can be a personal computer, a laptop computer, a notebook computer, a tablet, a tablet computer, a cellular telephone, a personal digital assistant, a wireless telephone, a wireless communication device, a personal communication device, a personal communications services device, a smart phone, a Smartphone, a mobile telephone, a hand-held device or computer, a palm-top device or computer, a watch, a telephone, a television, an interactive television, a digital television, a smart television or entertainment device, an internet-enabled television or entertainment device, or any other suitable device, which can be equipped to perform the functions described herein as being performed by the counterparty communication device 50.

In a preferred embodiment, the counterparty communication device 50 can include a central processing unit or device, an input device, a card reader, a barcode reader, a barcode scanner, a two-dimensional barcode reader, a two-dimensional barcode scanner, a QR code reader, a QR code scanner, an imaging device, a camera for obtaining an image of a barcode, a two-dimensional barcode, and/or a QR code, a two-dimensional image sensor, an account information data entry device, a card swiping device, a retinal scanning device, a fingerprint recognition device, a voice recognition device, a retinal scanner, a fingerprint device, a thumbprint device, a voice recognition device, a handprint recognition device, a handprint geometry recognition device, a facial feature recognition device, and/or any one or more of the biometric devices used to control access to a computer or a computer network which are known to those skilled in the art at the time of the filing of this patent application, a pointing device, a mouse, an output device, a database or a memory device and/or system, a random access memory (RAM) device, a read only memory (ROM) device, a video recording system or equipment, a camera(s), an audio recording system, device, or equipment, a microphone, a receiver or any number of receivers, a transmitter or any number of transmitters, a network interface device, an information or content gathering device, and/or any other devices, equipments, or systems, typically found in and/or utilized by any of the herein-described counterparty communication devices 50 described herein as being utilized in connection with the apparatus 100 of the present invention. In a preferred embodiment, the counterparty communication device 50 can also be equipped with a global positioning device which can be utilized to calculate, determine, or ascertain, the position or location of the counterparty communication device 50.

In a preferred embodiment, the counterparty communication device 50 can also contain, include, or be equipped with, a transmitter(s), a receiver(s), or any other network interface devices or equipment for facilitating bi-directional communication with, and/or data and/or information exchange with, the central processing computer/distributed ledger technology system 10.

In a preferred embodiment, the counterparty communication device 50 can also include, contain, or be equipped with a camera, a digital video recording system or equipment, a microphone, a digital audio recording system or equipment, or any another digital video and audio recording device or equipment or other digital media recoding equipment, that can allow the counterparty communication device 50 to record and store, for later play-back, any of the video and/or audio information which can or may be obtained using the apparatus 100 of the present invention. The counterparty communication device 50 can also be used to take or record a photograph, picture, video, a video clip, audio, or an audio clip, of the account holder or any other user, individual, or entity.

In a preferred embodiment, the counterparty communication device 50 can communicate, in bi-directional manner, with the central processing computer/distributed ledger technology system 10 and/or with any central processing computer 10A of the same and/or with any distributed ledger technology system 10B of the same.

In a preferred embodiment, any number of counterparty communication device 50 can be assigned to, utilized by, or associated with, any individual or account holder.

In a preferred embodiment, the apparatus 100 also includes a counterparty wallet 60. In a preferred embodiment, the counterparty wallet 60 can be a component of, and/or integrated within, the counterparty communication device 50 or the counterparty wallet 60 can be located separate and apart from and/or located externally from the counterparty communication device 50. In a preferred embodiment, the counterparty wallet 60 can store any data and/or information and/or payment account data and/or information needed, desired, to required, for making or effectuating real-time payments, peer-to-peer payments, and/or cryptocurrency payments. In a preferred embodiment, the counterparty wallet 60 can be any suitable and/or commercially available wallet or wallet software or software application.

In a preferred embodiment, the counterparty communication device 50 can be connected to or with, and/or linked to or with, the counterparty wallet 60, and/or the counterparty wallet 60 can be a component of, and/or integrated within, the counterparty communication device 50. In a preferred embodiment, the counterparty wallet 60 can be connected to, linked with, and/or wirelessly connected to or linked with, the central processing computer/distributed ledger technology system 10, and/or any central processing computer 10A or distributed ledger technology system 10B of the same, and/or the counterparty wallet 60 can be connected to, linked with, and/or wirelessly connected to or linked with, the counterparty communication device 50.

In a preferred embodiment, the counterparty wallet 60 can store account information, can store funds or cryptocurrencies of and/or for the counterparty, and/or can store any data and/or information regarding any accounts, funds, or cryptocurrencies, of or for the counterparty.

In a preferred embodiment, the apparatus 100 also includes a counterparty bank/financial records computer 70. In a preferred embodiment, the counterparty bank/financial records computer 70 can be a, or any, transaction processing computer(s) of, for, or associated with, be any bank, thrift, credit union, financial intermediary, or cryptocurrency exchange or wallet, which is used for storing funds or cryptocurrency or cryptocurrencies of or for the counterparty. In a preferred embodiment, the counterparty bank/financial records computer 70 can be connected to, or linked with the central processing computer/distributed ledger technology system 10, and/or any central processing computer 10A or distributed ledger technology system 10B of the same. In another preferred embodiment, the counterparty bank/financial records computer 70 can be connected to, or linked with, the counterparty wallet 60 and/or the counterparty communication device 50, either via a wired connection, a wireless connection, and/or any combination of the same.

In a preferred embodiment, the user communication device 20 and/or the user wallet 30 can be connected to, or linked with, the user bank/financial institution records computer 40 so as to allow the user communication device 20 and/or the user wallet 30 to communicate with the user bank/financial institution records computer 40 in a bi-directional manner.

In a preferred embodiment, the counterparty communication device 50 and/or the counterparty wallet 60 can be connected to, or linked with, the counterparty bank/financial institution records computer 70 so as to allow the counterparty communication device 50 and/or the counterparty wallet 60 to communicate with the counterparty bank/financial institution records computer 70 in a bi-directional manner.

In a preferred embodiment, the user communication device 20 and the counterparty communication device 50 can communicate with each other, can transmit signals, messages, data, and/or information to each other, and/or can receive signals, messages, data, and/or information from each other, in a bi-directional manner.

With reference once again to FIG. 1, the apparatus 100 can also include a real-time/peer-to-peer payment provider system 80, which can be any commercially available real-time or peer-to-peer payment system of platform which can be operated by private entities, by banks, by central banks, or by any other entities. In a preferred embodiment, the real-time/peer-to-peer payment provider system 80 can be operated by any financial services company, any bank, any financial institution, any financial intermediary, any brokerage firm, any financial services company or entity, any entity formed by one or more U.S. financial institutions and/or foreign financial institutions, the U.S. Federal Reserve System or a Federal Reserve Bank, and/or any other central bank of any country or territory. In a preferred embodiment, the central processing computer/distributed ledger technology system 10 and/or the central processing computer 10A or distributed ledger technology system 10B of the same, can be connected or linked to or with, or wirelessly linked to or with, the real-time/peer-to-peer payment provider system 80.

In a preferred embodiment, the central processing computer/distributed ledger technology system 10, and/or any central processing computer 10A or distributed ledger technology system 10B of the same, the user communication device 20, the user wallet 30, the user bank/financial institution records computer 40, the counterparty communication device 50, the counterparty wallet 60, the counterparty bank/financial records computer 70, can communicate with, can communicate with, can transmit signals, messages, data, and/or information to, and/or can receive signals, messages, data, and/or information from, the real-time/peer-to-peer payment provider system 80, in a bi-directional manner.

In a preferred embodiment, the apparatus 100 can be utilized in effectuating any type of real-time payment of peer-to-peer payment involving or in including any type or kind of currency, fiat currency, cryptocurrency, or any other medium of exchange.

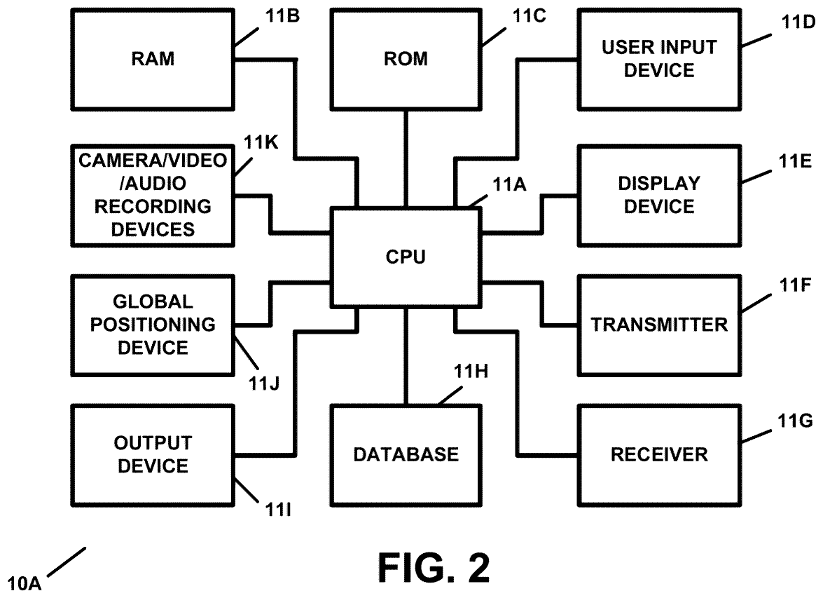

FIG. 2 illustrates a preferred embodiment of the central processing computer 10A of the central processing computer/distributed ledger technology system 10 of FIG. 1, in block diagram form. The central processing computer 10A, in the preferred embodiment, is a computer, a computer system, a group of computers, a network computer, or a network computer system, or any other communication device which can provide the functionality of, and which can be utilized as a central processing computer 10A. In the preferred embodiment, the central processing computer 10A can be adapted to process transaction authorization data and/or information for any of the accounts described herein in order to effectuate the processing and execution of real-time payments and/or peer-to-peer payments. In a preferred embodiment, the central processing computer 10A can also be an Internet computer, an Internet server computer, and/or a web site server computer. In the preferred embodiment, the central processing computer 10A includes a central processing unit or CPU 11A, which in the preferred embodiment, is a microprocessor. The CPU 11A may also be a microcomputer, a minicomputer, a macro-computer, and/or a mainframe computer, depending upon the application.

The central processing computer 10A also includes a random access memory device(s) 11B (RAM) and a read only memory device(s) 11C (ROM), each of which is connected to the CPU 11A, and a user input device 11D, for entering data, information, and/or commands, into the central processing computer 10A, which includes any one or more of a keyboard, a scanner, a card reader, a barcode reader, a barcode scanner, a two-dimensional barcode reader, a two-dimensional barcode scanner, a QR code reader, a QR code scanner, an imaging device, a camera for obtaining an image of a barcode, a two-dimensional barcode, and/or a QR code, a two-dimensional image sensor, an account information data entry device, a card swiping device, a touch screen, and/or a user pointing device, such as, for example, a mouse, a touch pad, and/or an audio input device and/or a video input device, a microphone or an audio recording device, a camera or a video recording device, and/or any device, electronic and/or otherwise which can be utilized for inputting and/or entering data and/or information, of any kind or type pertinent to the operation of the apparatus 100 of the present invention, into the central processing computer 10A. The input device 11D can also be any other input device(s) which are or can be utilized with or in connection with any of the central processing computer(s) 10A described herein as being utilized in connection with the apparatus 100 of the present invention. The input devices 11D are also connected to or with, or linked to or with, the CPU 11A. In a preferred embodiment, the input device 11D can also include a retinal scanner, a fingerprint recognition device, a voice recognition device, or any other type or kind of biometric device which can be used for determining whether or not a user or operator of the central processing computer 10A is an authorized user, individual, or person. The central processing computer 10A also includes a display device 11E for displaying data and/or information to a user or operator.

The central processing computer 10A also includes a transmitter(s) 11F, for transmitting signals and/or data and/or information, or a message(s), to any one or more of the user communication devices(s) 20, the counterparty communication device(s) 50, the user wallet(s) 30, the counterparty wallet(s) 60, the user bank/financial institution records computer(s) 40, the counterparty bank/financial records computer(s) 70, and/or the real-time/peer-to-peer payment provider system(s) 80, and/or any other central processing computer(s) 10A, described herein. The central processing computer 10 can also be equipped with transmitters, receivers, network interface devices, and/or any other appropriate hardware and/or software, so as to communicate, in a bi-directional manner with, so as to transmit signals, data, information, or a message to, and/or so as to receive signals, data, information, or a message from, any of aforementioned devices, computers, or systems 20, 30, 40, 50, 60, 70, and/or 80.

The central processing computer 10A also includes a receiver(s) 11G, for receiving signals and/or data and/or information, or a message(s), from any one or more of the user communication devices(s) 20, the counterparty communication device(s) 50, the user wallet(s) 30, the counterparty wallet(s) 60, the user bank/financial institution records computer(s) 40, the counterparty bank/financial records computer(s) 70, and/or the real-time/peer-to-peer payment provider system(s) 80, and/or any other central processing computer(s) 10A, described herein.

The central processing computer 10A also includes a database(s) 11H, which is also connected to or linked with the CPU 11A, which can contain and/or include any and/or all of the data and/or information needed or desired for performing any and/or all of the functions and/or functionality described herein as being performed by the apparatus 100 and method of the present invention and/or the central processing computer 10A.

In a preferred embodiment, the database 11H can contain and/or include any and/or all data and/or information disclosed and described as being stored in any of the respective central processing computer databases of the central processing computers disclosed and described in U.S. patent application Ser. No. 16/509,026, filed Jul. 14, 2019, U.S. patent application Ser. No. 16/029,550, and U.S. patent application Ser. No. 17/224,838, the subject matter and teachings of which are hereby incorporated by reference herein in their entirety. In a preferred embodiment, the database 11H can also contain or include any and/or all of the data, information, software, software applications, needed or desired to performing all of the herein-described functions and functionalities of the central processing computer(s) 10A, the central processing computer/distributed ledger technology system 10, and/or the apparatus 100.

For example, the database 11H contains and/or includes data and/or information regarding each user, individual, counterparty, or account holder, who or which utilizes the apparatus 100 of the present invention, and for each, his, her, or its, name, address, contact information, telephone number(s), cellular telephone number(s), wireless telephone number(s), personal communication device telephone number(s), e-mail address(es), IP address(es), text message number(s) or information, SMS message(s) or information, MMS message(s) or information, employer information, work information, emergency contact information, and/or any other contact or other information, account information, information regarding any and/or all accounts of the account holder, account numbers, account expiration dates, security codes or numbers, personal identification numbers (PINs), password(s), access code(s), social security numbers, account credit limit(s), account spending limit(s), account deductible(s), and/or any other data and/or information regarding each account holder and his, her, or its, accounts. In the case of individuals, the database 11H can also contain and/or include data and/or information regarding the account holder's relatives, friends, next of kin, or other contact information or emergency contact information.

The database 11H can also contain and/or include, for each user, individual, counterparty, or account holder, a photograph or video clip of the user, individual, counterparty, or account holder, data and/or information regarding a digital voiceprint of the user, individual, counterparty, or account holder, or data and/or information for verifying an identity of the user, individual, counterparty, or account holder, by his or her voiceprint, data and/or information regarding a retinal scan of the user, individual, counterparty, or account holder, or data and/or information for verifying an identity of the user, individual, counterparty, or account holder, by his or her retinal scan, data and/or information regarding a fingerprint of the user, individual, counterparty, or account holder, or data and/or information for verifying an identity of the user, individual, counterparty, or account holder, by his or her fingerprint, and/or any other data and/or information for identifying and identity of the user, individual, counterparty, or account holder, using biometric data and/or information.

In a preferred embodiment, the database 11H can also contain and/or include, for each user, individual, counterparty, or account holder, data and/or information regarding each user communication device 20 or counterparty communication device 50 which is or can be used by the user, individual, counterparty, or account holder, in utilizing the apparatus 100 and method of the present invention, including, but not limited to, data and/or information regarding an identification of each user communication device 20 or counterparty communication device 50, a description of, or type or kind of, same, the manufacturer, model number, and/or serial number or any other identification information, of, for, or regarding the same, the and/or the assigned telephone number, e-mail address, text messaging or SMS messaging number, MMS messaging number, and/or IP address, or any network identification information, of, for, or associated with, the same.

The database 11H can also contain and/or include, for each account serviced by the apparatus 100, data and/or information about the account holder, the account holder's account number(s), bank account number(s), real-time payment account number(s), peer-to-peer payment account number(s), healthcare payment account number(s), credit and/or account limit(s), spending limit(s), previous transactions, previous purchases, previous unauthorized transactions made on or involving the account or attempted to be made on or involving the account, previous unauthorized purchases made on or involving the account or attempted to be made on or involving the account, number of authorized and/or unauthorized transaction or purchases, account statements, historical account statements, and/or any other data and/or information needed, desired, and/or necessary, to manage and/or process an account transaction as described herein. The database 11H also contains and/or includes data and/or information regarding account statements, historical account statements, pending transactions, pending authorizations, and/or any other data and/or information regarding account activity and/or account activities.

The database 11H can also contain and/or include, for each account serviced by the apparatus 100, information regarding a user's, individual's, counterparty's, or account holder's, requests or instructions to receive alerts or alert messages regarding any transactions occurring on any of his, her, or its accounts.

The database 11H can also contain or include, for each account, the phone number, telephone number, uniform resource locator (url), or IP address of the respective central processing computer 10A which performs transaction authorization processing for the respective account. The database 11H can also contain or include a link(s) or hyperlink(s) to any central processing computer(s) 10A associated with any of the accounts held by, owned by, or associated with, each user, individual, counterparty, or account holder, serviced by the apparatus 100 of the present invention. The database 11H can also contain or include a link(s) or hyperlink(s) to any transaction page or web page or any transaction pages or web pages associated with the central processing computer(s) 10A associated with any of the accounts held by, owned by, or associated with, each user, individual, counterparty, or account holder.

The database 11H can also contain or include a link(s) or hyperlink(s) to each of the herein-described user communication devices 20, the counterparty communication devices 50, the user wallets 30, the counterparty wallets 60, the user bank/financial institution records computers 40, the counterparty bank/financial records computers 70, and/or the real-time/peer-to-peer payment provider systems 80, which are utilized in connection with the apparatus 100 of the present invention.

The database 11H can also contain and/or include data and/or information regarding each counterparty, merchant, store, wholesale store, retailer, retail store, vendor, supplier, customer, client, bank, financial institution, financial intermediary, service provider, goods provider, third party, or any other individual, person, or entity, who or which utilizes the apparatus 100 and method of the present invention and/or who or which is or can be a party to, enters into, engages in, or participates in, any transaction with any account holder who or which utilizes the apparatus 100 and method of the present invention (hereinafter referred to as “counterparty” or “merchant”). The database 11H can also contain and/or include, for each counterparty or merchant described herein, data and/or information regarding his, her, or its, name and/or counterparty identifier or counterparty identifying information and/or merchant identifier or merchant identifying information for or associated with each account type(s), account, bank account(s), credit card type(s), credit card account(s), charge card type(s), charge card account, debit card type(s), debit card account, bank account(s), checking account(s), savings account(s), payment account(s), electronic payment account(s), third party payment account(s), payment identifier, member identifier, customer identifier, client identifier, or other account information and/or counterparty identifier or counterparty identifying information and/or merchant identifier or merchant identifying information the respective counterparty or merchant has with, or has been assigned by or with, each account servicing entity, account servicing bureau, credit card serving company, charge card servicing company, debit card servicing company, bank, financial institution, financial intermediary, insurance company, healthcare insurance company, healthcare payer, or any other provider of any goods, products, or services (also referred to herein as “account servicing entity”). In this regard, the database 11H contains data and/or information regarding each respective account or identifier which the respective counterparty or merchant has with each account serving entity and/or the database 11H contains data and/or information regarding each respective account or identifier associated with each membership, subscription, or account or identifier, which the respective counterparty or merchant has with each account servicing entity or has been assigned by each account servicing entity.

The database 11H can also contain and/or include data and/or information regarding each of the herein-described counterparties or merchants, including, but not limited to, his, her, or its, name, address, contact information, which contact information can include, but is not limited to, telephone number(s), e-mail address(es), IP address(es), text message number(s) or information, SMS message(s) number(s) or information, MMS message(s) number(s) or information, cellular telephone number(s), and/or any other data and/or information for facilitating contact or communication with a respective counterparty or merchant.

The database 11H can also contain and/or include data and/or information regarding each the counterparty communication device 50 or counterparty communication devices 50, of, associated with, or used by any of the herein-described counterparties or merchants, including, but not limited to, for each counterparty communication device 50, which can include any of the herein-described counterparty communication devices 50, point-of-sale (POS) devices, point-of-transaction devices, transaction devices, transaction authorization devices, transaction authorization processing devices, or any other transaction devices, the type or kind or device, the manufacturer and model of same, and contact information, telephone number(s), e-mail address(es), IP address(es), text message number(s) or information, SMS message(s) number(s) or information, MMS message(s) number(s) or information, cellular telephone number(s), and/or any other data and/or information for facilitating contact or communication with the respective counterparty communication device 50.

In this regard, the database 11H can contain and/or include data and/or information regarding each counterparty communication device 50 utilized by each respective counterparty or merchant, including, but not limited to, for each communication device 50, whether the communication device 50 be any of the devices described herein as being a counterparty communication device 50, or a point-of-sale device, a point-of-transaction device, a card swiping device equipped for use in processing transactions or for processing information for processing transaction payments, a transaction device, a transaction authorization device, a transaction authorization processing device, or any other transaction device or other device, the telephone number(s), e-mail address(es), IP address(es) or information, text messaging number(s) or information, SMS messaging number(s) or information, MMS messaging number(s) or information, and/or any other contact information, for or associated with each counterparty communication device 50 which can or may be utilized for facilitating communication with the respective counterparty communication device 50 and/or which can or may be used for facilitating communication with and/or between the respective counterparty communication device 50 and any of the central processing computer(s) 10A, the user communication device(s) 20, described herein.

The database 11H can also contain and/or include, for each of the herein-described counterparties or merchants, any data and/or information regarding any of the herein-described counterparty transaction identifying information and/or merchant transaction identifying information, and/or any other data and/or information described herein as being contained in or included in same for that respective counterparty or merchant. The database 11H can also contain and/or include, for each of the herein-described counterparties or merchants, data and/or information regarding any payment account information, telephone number(s), e-mail address(es), IP address(es), text message number(s) or information, SMS message(s) number(s) or information, MMS message(s) number(s) or information, wireless telephone number(s), personal communication device telephone number(s), point-of-sale transaction devices telephone number(s), e-mail address(es), IP address(es), text message number(s) or information, SMS message(s) number or information, MMS message(s) number or information, emergency contact information, and/or any other contact or other information, account information, information regarding any and/or all accounts of the respective counterparty or merchant, account number(s), account identifier(s), account expiration dates, security codes or numbers, personal identification numbers (PINs), password(s), access code(s), payment instructions, employer identification numbers, account credit limit(s), account spending limit(s), account deductible(s), and/or any other data and/or information regarding each counterparty or merchant and/or his, her, or its, respective accounts.

The database 11H can also contain and/or include, for each of the herein-described counterparties and/or merchants, a description of the respective counterparty or merchant, a photograph or video clip of the counterparty's or merchant's principal(s), employee(s), or agent(s), authorized to engage in transactions or to utilize the apparatus 100, data and/or information regarding a digital voiceprint of the of the counterparty's or merchant's principal(s), employee(s), or agent(s), authorized to engage in transactions or to utilize the apparatus 100, or data and/or information for verifying an identity of the counterparty's or merchant's principal(s), employee(s), or agent(s), by his or her voiceprint, data and/or information regarding a retinal scan of the counterparty's or merchant's principal(s), employee(s), or agent(s), or data and/or information for verifying an identity of the counterparty's or merchant's principal(s), employee(s), or agent(s), by his or her retinal scan, data and/or information regarding a fingerprint of the counterparty's or merchant's principal(s), employee(s), or agent(s), or data and/or information for verifying an identity of the counterparty's or merchant's principal(s), employee(s), or agent(s), by his or her fingerprint, and/or any other data and/or information for identifying and identity of the counterparty's or merchant's principal(s), employee(s), or agent(s), using biometric data and/or information.

In a preferred embodiment, the database 11H can also contain and/or include, for each of the herein-described counterparties or merchants, data and/or information regarding each counterparty communication device 50 which is or can be used by the respective counterparty or merchant in utilizing the apparatus 100 and method of the present invention, including, but not limited to, data and/or information regarding an identification of each counterparty communication device 50, a description of, or type or kind of, the counterparty communication device 50, manufacturer, model number, and/or serial number or any other identification information, of, for, or regarding, the c counterparty communication device 50, and/or the assigned telephone number, e-mail address, text messaging or SMS messaging number, MMS messaging number, and/or IP address, or any network identification information, of, for, or associated with, the respective counterparty communication device 50.

The database 11H can also contain and/or include, for each counterparty or merchant account serviced by the apparatus 100, information about the respective counterparty or merchant, the counterparty's or merchant's account number(s), bank account number(s), credit and/or account limit(s), spending limit(s), previous transactions, previous purchases, previous unauthorized transactions made on the account or attempted to be made on the account, previous unauthorized purchases made on the account or attempted to be made on the account, number of authorized and/or unauthorized transaction or purchases, account statements, historical account statements, and/or any other data and/or information needed, desired, and/or necessary, to manage and/or process an account transaction as described herein. The database 11H also contains and/or includes data and/or information regarding account statements, historical account statements, pending transactions, pending authorizations, and/or any other data and/or information regarding account activity and/or account activities for any counterparty or merchant accounts.

The database 11H can also contain and/or include, for each account serviced by the apparatus 100, information regarding a counterparty's or merchant's requests or instructions to receive alerts or alert messages regarding any transactions occurring on any of his, her, or its, accounts.

The database 11H can also contain or include, for each counterparty or merchant account, the phone number or IP address of the respective central processing computer 10A which performs transaction authorization processing for the respective account. The database 11H can also contain or include a link(s) or hyperlink(s) to any central processing computer(s) 10A associated with any of the counterparty or merchant accounts held by, owned by, or associated with, each counterparty or merchant serviced by the apparatus 100 of the present invention. The database 11H can also contain or include a link(s) or hyperlink(s) to any transaction page or pages associated with the central processing computer(s) 10A associated with any of the accounts held by, owned by, or associated with, each counterparty or merchant.

The database 11H can also contain or include, for each account, the times or hours of operation for each of the respective central processing computer(s) 10A which perform transaction authorization processing for any of the user, individuals, counterparties, and/or accounts, serviced by the apparatus 100 of the present invention.

The database 11H can also contain or include, for each user, individual, or counterparty, and/or for each account, data and/or information regarding specific limitations and/or restrictions which may be placed on each user, individual, or counterparty, and/or on each account, which may be pre-selected and/or programmed by the user, individual, or counterparty, and/or which may include limitations and/or restrictions on the usage of the account or any account numbers associated with the account and/or which may be placed on the account by the entity which issued the account or which services the account. The limitations and/or restrictions may include the types of transactions which are allowed and/or authorized, the goods and/or services which may be purchased with the account, the vendors, stores and/or service providers, which may be authorized to accept the payment via the account, limits on the dollar or other monetary amounts of transactions pertaining to each authorized vendor, seller, and/or service provider, daily spending limits, and/or the geographical area or location wherein authorized account use may be limited, and/or authorized times for account usage, such as, but not limited to, specific days, dates, time of day, time of month, year, and/or any other time of use, and/or any other limitation and/or restriction regarding amount of transaction, parties involved, geographical area, and/or times of allowed usage.

The database 11H can also contain or include, for each account, data and/or information regarding transactions processed on or involving the account. The data and/or information can also include, for each of any number of transactions for each or any number of accounts, a picture, a photograph, or a video clip, of the account holder or other user or individual involved in a transaction, and/or audio information, a voice message or sample, and/or a voice print, which is a digital representation of a voice message or sample, obtained from the account holder or other user or individual involved in the transaction, for each of any number of transactions processed by the central processing computer 10A. For example, for any given account, the central processing computer 10A can store information for any number of transactions occurring on a given account. In this manner, a picture, a photograph, or a video clip, of the user, individual, or counterparty, involved in a transaction, and/or audio information, a voice message or sample, and/or a voice print, which is a digital representation of a voice message or sample, obtained from the user, individual, or counterparty, involved in the transaction, can be stored for any number of transactions on a respective account, in order to perform identity verification for an user, individual, or counterparty, involved in a transaction.

The database 11H can also contain or include copies of any of the images, digital copies, photographs, or pictures, of any of the respective documents, authorization forms, checks, forms, receipts, request forms, or other entities, which have been, or which were, offered, presented, submitted, processed, involved, or used, in or involving, or related to, a transaction or transactions on or involving any of the accounts serviced by the apparatus 100.

In another preferred embodiment, the database 11H can also contain any electronic healthcare records, electronic medical records, electronic dental records, electronic pharmacy records, electronic behavioral health records, and/or any other healthcare information, and/or healthcare-related information, regarding any users, individuals, counterparties, and/or others, who or which utilized the apparatus 100 and method of the present invention. In this regard, Applicant also hereby incorporates by reference herein the subject matter and teachings of each of U.S. patent application Ser. No. 17/224,838, filed Apr. 7, 2021, and entitled “APPARATUS AND METHOD FOR PROVIDING HEALTHCARE SERVICES REMOTELY OR VIRTUALLY WITH OR USING AN ELECTRONIC HEALTHCARE RECORD AND/OR A COMMUNICATION NETWORK”, U.S. Provisional Patent Application Ser. No. 63/634,841, filed Apr. 16, 2024, and entitled “APPARATUS AND METHOD FOR PROVIDING HEALTHCARE SERVICES REMOTELY OR VIRTUALLY WITH OR USING AN ELECTRONIC HEALTHCARE RECORD AND/OR A COMMUNICATION NETWORK AND/OR WITH OR USING A PERSONAL MONITORING DEVICE”, filed April 2024, and U.S. Provisional Patent Application Ser. No. 63/643,488, filed May 7, 2025, and entitled “APPARATUS AND METHOD FOR PROVIDING HEALTHCARE SERVICES REMOTELY OR VIRTUALLY WITH OR USING AN ELECTRONIC HEALTHCARE RECORD AND/OR A COMMUNICATION NETWORK AND/OR WITH OR USING A PERSONAL MONITORING DEVICE”, the subject matter and teachings of all of which herein-identified applications are hereby incorporated by reference herein in their entirety.

In a preferred embodiment, the database 11H can contain and/or include any of the data and/or information, and/or software programs or software applications, described in any and/or all of the databases, central processing computers, computers, and/or communication devices described in any and/or all of the patent applications described herein and the subject matter and teachings of which are described herein in this patent application.

Applicant also hereby incorporates by reference herein the subject matter and teachings of U.S. Provisional Patent Application Ser. No. 62/716,779, filed Aug. 9, 2018, and entitled “APPARATUS AND METHOD FOR PROVIDING TRANSACTION SECURITY AND/OR ACCOUNT SECURITY”, U.S. Provisional Patent Application Ser. No. 62/715,110, filed Aug. 6, 2018, and entitled “APPARATUS AND METHOD FOR PROVIDING TRANSACTION SECURITY AND/OR ACCOUNT SECURITY”, U.S. patent application Ser. No. 14/458,316, filed Aug. 13, 2014, and entitled “APPARATUS AND METHOD FOR PROVIDING TRANSACTION SECURITY AND/OR ACCOUNT SECURITY”, U.S. patent application Ser. No. 14/328,434, filed Jul. 10, 2014, and entitled “APPARATUS AND METHOD FOR PROVIDING TRANSACTION SECURITY AND/OR ACCOUNT SECURITY”, U.S. patent application Ser. No. 14/283,820, filed May 21, 2014, and entitled “APPARATUS AND METHOD FOR PROVIDING TRANSACTION SECURITY AND/OR ACCOUNT SECURITY”, U.S. patent application Ser. No. 14/289,673, filed May 29, 2014, and entitled “APPARATUS AND METHOD FOR PROVIDING TRANSACTION SECURITY AND/OR ACCOUNT SECURITY”, U.S. Provisional Patent Application Ser. No. 62/035,541, filed Aug. 11, 2014, and entitled “APPARATUS AND METHOD FOR PROVIDING TRANSACTION SECURITY AND/OR ACCOUNT SECURITY”, U.S. Provisional Patent Application Ser. No. 61/880,757, filed Sep. 20, 2013, and entitled “APPARATUS AND METHOD FOR PROVIDING TRANSACTION SECURITY AND/OR ACCOUNT SECURITY”, U.S. Provisional Patent Application Ser. No. 61/957,360, filed Jul. 1, 2013, and entitled “APPARATUS AND METHOD FOR PROVIDING TRANSACTION SECURITY AND/OR ACCOUNT SECURITY”, and U.S. Provisional Patent Application Ser. No. 61/956,801, filed Jun. 17, 2013, and entitled “APPARATUS AND METHOD FOR PROVIDING TRANSACTION SECURITY AND/OR ACCOUNT SECURITY”, the subject matter and teachings of which are hereby incorporated by reference herein in their entirety, so as to incorporate by reference herein any and/or all data and/or information described as being stored in each of the databases, computers, and/or communication devices described herein.

It is envisioned that a user, individual, or counterparty, or an authorized user or individual, of a respective account can submit a photograph or picture or his or her face which can be stored in the database 11H as a “file photograph” or a “file picture” associated with the respective account. In a preferred embodiment, the “file photograph” or “file picture” can be stored as a digital file. It is also envisioned that the user, individual, or counterparty or authorized user or individual can also submit a pre-recorded voice message which can be digitized into a voice print which can be stored as a “file voiceprint”.

In processing a transaction involving a respective account, the central processing computer 10A can process a picture, a photograph, or a video clip, of the account holder or user or individual involved in the transaction, and/or audio information, a voice message or sample, and/or a voice print, obtained from the user, individual, or counterparty involved in the transaction using the respective “file photograph”, “file picture”, and/or “file voice print”, and/or can provide the “file photograph”, “file picture”, and/or “file voice print”, to a user communication device 20 or to a counterparty communication device 50 so that the operator of same can use the same in verifying that the user, individual, or counterparty is the account holder associated with the account or is an authorized user or individual associated with the account.

The database 11H can also contain or include account statements or periodic transaction records, for each of the account holder accounts or user accounts serviced by the apparatus 100. The database 11H can also contain or include account statements or periodic transaction records for each of the counterparty accounts or merchant accounts serviced by the apparatus 100.

The database 11H can also contain and/or include, for each account holder account or user account, and for each user communication device(s) 20 associated with the user, individual, or counterparty, data and/or information regarding account statements, historical statements, periodic transaction records, and/or any other data and/or information regarding past transactions which have occurred on the respective account, data and/or information regarding the user communication device 20 utilized in connection with a particular transaction, and/or any information for providing periodic transaction statements showing or listing activities, and/or attempted transactions which have occurred on an account, the user communication device 20 involved, the counterparty communication device 50 involved, the date and time of the transaction, the user, individual, or counterparty involved in the transaction, and/or the counterparty or merchant involved in the transaction, and/or the transaction amount and the subject of the transaction, such as, but not limited to, the products, goods, or services, which are the subject of the transaction.

The database 11H can also contain and/or include data and/or information for providing periodic transaction reports showing or listing, for each user communication device 20 associated with an account, or attempted activities, on or involving the account, including transactions which have occurred on or using the user communication device 20, including, but not limited to, data and/or information regarding the transaction, the counterparty communication device 50 involved, the date and time of the transaction, the account holder or user involved in the transaction, or the counterparty or merchant involved in the transaction, and/or the transaction amount and the subject of the transaction, such as, but not limited to, the products, goods, or services, which are the subject of the transaction or activities that were authorized or completed and/or which were not authorized or disallowed.

The database 11H can also contain or include, for each user, individual, or counterparty, and/or for each account, data and/or information regarding a barcode containing information regarding the user, individual, or counterparty or the account number associated with the respective user, individual, or counterparty, account, data and/or information regarding a two-dimensional barcode containing information regarding the user, individual, or counterparty, and/or the account number associated with the respective account, and/or data and/or information regarding a QR code or Quick Response code containing information regarding the user, individual, or counterparty, and/or the account number associated with the respective account. The database 11H can also contain data and/or information and/or software processing routines for scanning, reading, and/or deciphering, data and/or information contained in a barcode, a two-dimensional barcode, and/or a QR code or Quick Response code.

In a preferred embodiment, the database 11H contains and/or includes data and/or information regarding each merchant, vendor, seller, supplier, or any provider of any good(s), product(s), or service(s), service provider, professional service provider, or any other person, individual, business, or other entity (hereinafter also referred to as a “counterparty”) who or which utilizes the apparatus 100 of the present invention, and for each counterparty, his, her, or its, name, address, contact information, telephone number(s), cellular telephone number(s), wireless telephone number(s), personal communication device telephone number(s), e-mail address(es), IP address(es), text message number(s) or information, SMS message(s) or information, SMS message(s) or information, and/or any other contact or other information, account information for any account into which payments are received for the counterparty and/or any other accounts of the counterparty, information regarding any and/or all accounts of the counterparty, account numbers, account expiration dates, security codes or numbers, personal identification numbers (PINs), password(s), access code(s), social security numbers or employer identification numbers, account credit limit(s), account spending limit(s), account deductible(s), and/or any other data and/or information regarding each counterparty and his, her, or its, accounts. In the case of individuals, the database 11H can also contain and/or include data and/or information regarding the counterparty's contact information or emergency contact information.

The database 11H can also contain and/or include, for each counterparty or for each user of an account of a counterparty, a description of the counterparty, an employee or agent of the counterparty, or user, a photograph or video clip of the counterparty, an employee or agent of the counterparty, or user, data and/or information regarding a digital voiceprint of the counterparty, an employee or agent of the counterparty, or user, or data and/or information for verifying an identity of the counterparty, an employee or agent of the counterparty, or user, by his or her voiceprint, data and/or information regarding a retinal scan of the counterparty, an employee or agent of the counterparty, or user, or data and/or information for verifying an identity of the counterparty, an employee or agent of the counterparty, or user, by his or her retinal scan, data and/or information regarding a fingerprint of the counterparty, an employee or agent of the counterparty, or user, or data and/or information for verifying an identity of the counterparty, an employee or agent of the counterparty, or user, by his or her fingerprint, and/or any other data and/or information for identifying and identity of the counterparty, an employee or agent of the counterparty, or user, using biometric data and/or information.

In a preferred embodiment, the database 11H can also contain and/or include, for each counterparty, an employee or agent of the counterparty, or user, data and/or information regarding each counterparty communication device 50 which is or can be used by the counterparty, an employee or agent of the counterparty, or user, in utilizing the apparatus 100 and method of the present invention, including, but not limited to, data and/or information regarding an identification of each counterparty communication device 50, a description of, or type or kind of, the counterparty communication device 50, manufacturer, model number, and/or serial number or any other identification information, of, for, or regarding, the counterparty communication device 50, and/or the assigned telephone number, e-mail address, text messaging or SMS messaging number, MMS messaging number, and/or IP address, or any network identification information, of, for, or associated with, the counterparty communication device 50.

The database 11H can also contain and/or include, for each account serviced by the apparatus 100 for the counterparty, information about the counterparty, the counterparty's account number(s) for any and/or all accounts of the counterparty, previous transactions made or involving the account, previous unauthorized transactions made on or involving the account or attempted to be made on or involving the account, previous unauthorized sales made on or involving the account or attempted to be made on or involving the account, number of authorized and/or unauthorized transactions or sales, account statements, historical account statements, and/or any other data and/or information needed, desired, and/or necessary, to manage and/or process an account transaction for a counterparty as described herein. The database 11H can also contain and/or include data and/or information regarding account statements, historical account statements, pending transactions, pending authorizations, and/or any other data and/or information regarding account activity and/or account activities for the counterparty.

The database 11H can also contain and/or include, for each account serviced by the apparatus 100 for a counterparty, information regarding an counterparty's requests or instructions to receive alerts or alert messages regarding any transactions occurring on any of his, her, or its accounts.

The database 11H can also contain or include, for each account serviced by the apparatus 100, the phone number or IP address of the respective central processing computer 10A which performs transaction authorization processing for the respective account. The database 11H can also contain or include a link(s) or hyperlink(s) to any central processing computer(s) 10A associated with any of the accounts held by, owned by, or associated with, each counterparty serviced by the apparatus 100 of the present invention. The database 11H can also contain or include a link(s) or hyperlink(s) to any transaction page or web page or pages or web pages associated with the central processing computer(s) 10A associated with any of the accounts held by, owned by, or associated with, each counterparty who or which utilizes the apparatus 100 of the present invention.

The database 11H can also contain or include, for each account of a counterparty, the times or hours of operation for each of the respective central processing computer(s) 10A which perform transaction authorization processing for any of the accounts serviced for the counterparty by the apparatus 100 of the present invention.

The database 11H can also contain or include, for each account of a counterparty, data and/or information regarding specific limitations and/or restrictions which may be placed on a particular account, which may be pre-selected and/or programmed by the counterparty and/or which may include limitations and/or restrictions on the usage of the account or any bank account(s), or any cards or account numbers, associated with the account and/or which may be placed on the account by the entity which issued the account or which services the account. The limitations and/or restrictions may include the types of transactions which are allowed and/or authorized, the goods and/or services which may be sold with the account, the employees or agents of the counterparty, or other users, who or which may be authorized to perform transactions on or involving the account, limits on the dollar or other monetary amounts of transactions which can be entered into involving the account, daily use limitations, and/or the geographical area or location wherein authorized account use may be limited, and/or authorized times for account usage, such as, but not limited to, specific days, dates, time of day, time of month, year, and/or any other time of use, and/or any other limitation and/or restriction regarding amount of transaction, parties involved, geographical area, and/or times of allowed usage.

The database 11H can also contain or include, for each account of a counterparty, data and/or information regarding transactions processed on or involving the account. The data and/or information can also include, for each of any number of transactions for each or any number of accounts of a counterparty, a picture, a photograph, or a video clip, of the counterparty, or of an employee or agent of same, or other authorized user, who or which can be involved in a transaction on or involving the account, and/or audio information, a voice message or sample, and/or a voice print, which is a digital representation of a voice message or sample, obtained from the counterparty, or of an employee or agent of same, or other authorized user, who or which can be involved in a transaction on or involving the account, for each of any number of transactions processed by the central processing computer 10A. For example, for any given account, the central processing computer 10A can store information for any number of transactions occurring on a given account. In this manner, a picture, a photograph, or a video clip, of the counterparty, or of an employee or agent of same, or other authorized user, who or which can be involved in a transaction on or involving the account, and/or audio information, a voice message or sample, and/or a voice print, which is a digital representation of a voice message or sample, obtained from the counterparty, or of an employee or agent of same, or other authorized user, who or which can be involved in a transaction on or involving the account, can be stored for any number of transactions on a respective counterparty account, in order to perform identity verification for a counterparty or other user or individual involved in a transaction on or involving an account of the counterparty.

The database 11H can also contain or include copies of any of the images, digital copies, photographs, or pictures, of any of the respective documents, authorization forms, checks, forms, receipts, request forms, or other entities, which have been, or which were, offered, presented, submitted, processed, involved, or used, in or involving, or related to, a transaction or transactions on or involving any of the accounts of a counterparty serviced by the apparatus 100.

It is envisioned that a counterparty, or an employee or agent of the counterparty, or any other authorized user or individual, of a respective counterparty account can submit a photograph or picture or his or her face which can be stored in the database 11H as a “file photograph” or a “file picture” associated with the respective counterparty account. In a preferred embodiment, the “file photograph” or “file picture” can be stored as a digital file. It is also envisioned that the counterparty, or an employee or agent of the counterparty, or any other authorized user or individual, of a respective counterparty account can also submit a pre-recorded voice message which can be digitized into a voice print which can be stored as a “file voiceprint”.

In processing a transaction involving a respective counterparty account, the central processing computer 10A can process a picture, a photograph, or a video clip, of the counterparty, or an employee or agent of the counterparty, or any other authorized user or individual involved in the transaction, and/or audio information, a voice message or sample, and/or a voice print, obtained from the counterparty, or an employee or agent of the counterparty, or any other authorized user or individual involved in the transaction using the respective “file photograph”, “file picture”, and/or “file voice print”, and/or can provide the “file photograph”, “file picture”, and/or “file voice print”, to a user communication device 20 so that the account holder or other authorized user of the account holder's account can use same in verifying that the counterparty, or an employee or agent of the counterparty, or any other authorized user or individual is, in fact, the counterparty, or an employee or agent of the counterparty, or any other authorized user or individual with whom the account holder or other authorized user or individual is intending to conduct the transaction.

The database 11H can also contain or include, for each counterparty account, data and/or information regarding account statements, historical statements, periodic transaction records, and/or any other data and/or information regarding past transactions and/or activities, and/or attempted transactions or attempted activities, on or involving the counterparty account, including transactions or activities that were authorized or completed and/or which were not authorized or disallowed.

The database 11H can also contain or include, for each counterparty account, data and/or information regarding a barcode containing information regarding the counterparty account number associated with the respective counterparty account, data and/or information regarding a two-dimensional barcode containing information regarding the account number associated with the respective counterparty account, and/or data and/or information regarding a QR code or Quick Response code containing information regarding the account number associated with the respective counterparty account. The database 11H can also contain data and/or information and/or software processing routines for scanning, reading, and/or deciphering, data and/or information contained in a barcode, a two-dimensional barcode, and/or a QR code or Quick Response code.

The database 11H can also contain or include, for each account held by a user, individual, or counterparty, who or which is serviced by the apparatus 100 of the present invention, data and/or information regarding or pertaining to the account, the type of the account, such as, but not limited to, whether the account is a bank account, a merchant account, a credit account, a credit card account, a charge card account, a charge account, a debit card account, or a debit account, or a bank account, a checking account, or a savings account, or a brokerage account, a pension account, an individual retirement account (IRA), or a self-employed pension (SEP) account, or a “smart” card account, a currency card account, a healthcare account, a Medicare account, or a Medicaid account, or an employee benefits account, a cafeteria account, or a spending account, or a subscription account for any goods, products, or services, or an insurance account, a healthcare insurance account, a healthcare spending account, a life insurance account, or a disability insurance account, or a tuition account, a pharmacy account, or credit report account, a financial account, an electronic money account, or an electronic cash account, or a communication account, a telephone account, a wireless communication device account, a non-wireless communication device account, a cellular communication device account, a cellular telephone account, an Internet account, or an Internet service provider account, an electronic signature account, an e-mail account, a membership account, a text messaging account, a customer loyalty membership account, a club membership account, a social network membership account, or any other account of the type or variety described herein, and/or the account number or an account identifier for the account, the telephone number, e-mail address, or the IP address, identified with the account, and/or any and/or all data and/or information regarding the account issuer or the account service provider, servicing entity, or transaction authorizing processing provider or entity, including, but not limited to, the telephone number, e-mail address, or the IP address, for, associated with, or identified with, the account, and/or with the account issuer or the account service provider, servicing entity, or transaction authorizing processing provider or entity, or any other entity which issued and/or which services the account, whether the account issuer or the account service provider, servicing entity, or transaction authorizing processing provider or entity, or any other entity, is the VISA®, MASTERCARD®, DISCOVER®, or AMERICAN EXPRESS®, financial services company, or any bank, financial institution, financial intermediary, brokerage firm, financial services company or entity, an entity formed by U.S. financial institutions and/or foreign financial institutions, the U.S. Federal Reserve System, and/or any other central bank of any country or territory, and/or an insurance company, or any provider of any good(s), product(s), or service(s), or any other entity which issues or which services any account described herein, or any entity which provides transaction authorization processing for or which provides transaction authorization processing services for any of the herein-described accounts, the telephone number, uniform resource locator (url), IP address, e-mail address, and/or web address, of the respective central processing computer 10A which processes transaction authorization requests and/or transactions for, on, or involving the respective account, and/or the telephone number, uniform resource locator (url), IP address, e-mail address, and/or web address, needed to perform transaction authorization processing by the pertinent and/or the respective central processing computer 10A which processes transaction authorization requests and/or transactions for, on, or involving the respective account.

In this regard, the data and/or information regarding the account also includes any needed and/or desired data and/or information for allowing an user communication device 20 to access and/or communicate with the respective central processing computer 10A, which performs transaction authorization processing for a transaction on or involving the account, in order to obtain transaction authorization processing for a transaction. For example, in the case of a bank account, the data and/or information for that account can include a telephone number or IP address which be utilized by the user communication device 20 to access the central processing computer 10A, which performs transaction authorization processing for that bank account, with his or her communication device 20. The data and/or information for that bank account can include a telephone number or IP address which be utilized by the user communication device 20 to access and/or communicate with, automatically and/or otherwise, the central processing computer 10A, which performs transaction authorization processing for that credit account.

The database 11H can also contain and/or include, for each account, any access code(s), security code(s), password(s), or any other data and/or information regarding the account. The database 11H can also contain and/or include, for each account and for each user communication device 20 registered to be utilized with the account, the manufacturer, model number, and/or serial number, and/or telephone number, e-mail address, IP address, text messaging number, SMS messaging number, MMS messaging number, or any other identifying information and/or contact information for or regarding each such user communication device 20.

The database 11H can also contain or include, for each user, individual, or counterparty, or for account held by each user, individual, or counterparty which is serviced by the apparatus 100 of the present invention, data and/or information regarding or pertaining to the account, the type or kind of the account, such as, but not limited to, a financial account or a non-financial account, or any other of the types or variety of accounts described herein, the account number or an account identifier for the account, the telephone number, e-mail address, or the IP address, identified with the account, and/or data and/or information for or regarding the account issuer or the account service provider, servicing entity, or transaction authorizing processing provider or entity, including, but not limited to, the telephone number, e-mail address, or the IP address, for, associated with, or identified with, the account, and/or with the account issuer or the account service provider, servicing entity, or transaction authorizing processing provider or entity, or any other entity which issued and/or which services the account.

The database 11H can also contain or include, for each account held by the counterparty, data and/or information regarding the counterparty communication device(s) 50 associated with the counterparty account, and the telephone number, e-mail address, or the IP address, or other contact information, identified with each counterparty communication device 50. In this regard, the data and/or information regarding the counterparty account also includes any needed and/or desired data and/or information for allowing any respective central processing computer 10A to access and/or communicate with the respective counterparty communication device 50 in or during, or as part of, a processing of a transaction authorization processing operation or activity and/or to obtain transaction authorization processing information from the counterparty communication device 50 and/or to transmit any of the herein-described messages, transaction authorized messages, transaction not authorized messages, or any other messages, signals, data and/or information, from the pertinent central processing computer 10A, to the counterparty communication device 50. For example, in the case of a real-time payment or a peer-to-peer payment transaction, the data and/or information for the counterparty's account can include a telephone number or IP address which can be utilized by the central processing computer 10A, which performs the transaction processing for that transaction, to access and/or communicate with the counterparty communication device 50. The data and/or information for that account can include a telephone number or IP address which be utilized by the central processing computer 10A, which performs transaction authorization processing for that transaction, to access and/or communicate with, automatically and/or otherwise, the counterparty communication device 50.

The database 11H can also contain and/or include, for each user, individual, or counterparty, or for each account, any access code(s), security code(s), password(s), or any other data and/or information regarding the user, individual, or counterparty, or the account. The database 11H can also contain and/or include, for each account and for each counterparty communication device 50 registered to be utilized with the counterparty account, the manufacturer, model number, and/or serial number, and/or telephone number, e-mail address, IP address, text messaging number, SMS messaging number, MMS messaging number, or any other identifying information and/or contact information for or regarding each such counterparty communication device 50.

The database 11H can also contain or include any of the data and/or information described herein as being stored in the databases of any of the user communication devices 20 and counterparty communication devices 50 described herein.

The database 11H can also contain or include any and/or all data and/or information and/or any software programs, routines, and/or software applications or “apps”, needed or desired for performing any and/or of the processing routines, functions, and/or functionality, described herein as being provided by or performed by the apparatus 100 of the present invention and/or by the central processing computer(s) 10A, the central processing computer/distributed ledger technology system 10, the user communication devices(s) 20, and/or the counterparty communication device(s) 50.

The database 11I can also contain or include data and/or information regarding the various users, individuals, account holders, and/or counterparties, who or which utilize the apparatus 100 and method of the present invention, including, but limited to, for each of the users, individuals, account holders, and/or counterparties, their respective accounts, transactions occurring on and/or relating to, each of their respective accounts, parties and counterparties involved in each transaction occurring on and/or relating to, each of their respective accounts, and for each transaction, the database 11I can include information regarding the respective users, individuals, account holders, and/or counterparties, involved, their names or other identifying information, the date and time of each transaction, the transaction amount, a description of the goods or services which are the subject of the transaction, the IP address(es) or position of location information of or for each user communication device 20 and/or counterparty communication device 50 utilized in the transaction, and/or a transaction identification number.

The database 11H can also contain or include any and/or all data and/or information and/or artificial intelligence (AI) software programs or AI software applications for processing any transactions between any of the respective users, individuals, account holders, and/or counterparties, using information regarding past transactions of any type or kind for any of the respective users, individuals, account holders, and/or counterparties, and/or any past transactions of any type or kind between any party or parties, which can involve any of the respective users, individuals, account holders, and/or counterparties, with any other users, individuals, account holders, and/or counterparties.

The database 11H can also contain or include any and/or all data and/or information and/or any software programs, routines, and/or software applications or “apps”, needed or desired for performing any and/or of the processing routines, functions, and/or functionality, described herein as being provided by any of the herein-described user communication devices 20 or counterparty communication devices 50, which such data and/or information and/or software applications or “apps”, being downloadable to the user communication device(s) 20 and/or counterparty communication device(s) 50 if and when needed or desired.

In a preferred embodiment, the database 11H can also contain or include any and/or all data and/or information needed, desired, or utilized, by the apparatus 100, or by the central processing computer(s) 10A and/or by the user communication devices(s) 20, the counterparty communication device(s) 50, described herein for or in performing any and/or all of the processing routines, operations, functions, and/or functionality, described herein as being performed by the apparatus 100 and method of the present invention.

The central processing computer 10A also includes an output device 11I, which is also connected to the CPU 11A, for outputting any data and/or information, described herein. In the preferred embodiment, the output device 11I can be a printer, a display, a transmitter, a modem, and/or any other device which can be used to output data or information.

The central processing computer 10A can also be equipped with a global positioning device 11J which can be connected to the CPU 11A and which can be utilized to calculate, determine, or ascertain, the position or location of the central processing computer 10A.

The central processing computer 10A can also include a video and/or audio recording device 11K which, in a preferred embodiment, can include a camera, a video recoding device, a microphone, and/or an audio recording device. The video and/or audio recording device 11K can be utilized to take a picture, record video, record a video clip, record sound, record audio, or record an audio clip, of a user of the central processing computer 10A and/or to record any picture, a sound or voice, video information, or audio information at the central processing computer 10A.

In a preferred embodiment, the central processing computer 10A, the central processing computer/distributed ledger technology system 10, and/or the a distributed ledger technology system 10B, can perform any of the functions and/or functionalities described in any and/or all of the patent applications and/or provisional patent applications and their respective subject matter and teachings of the same which are incorporated by reference herein in their entirety.

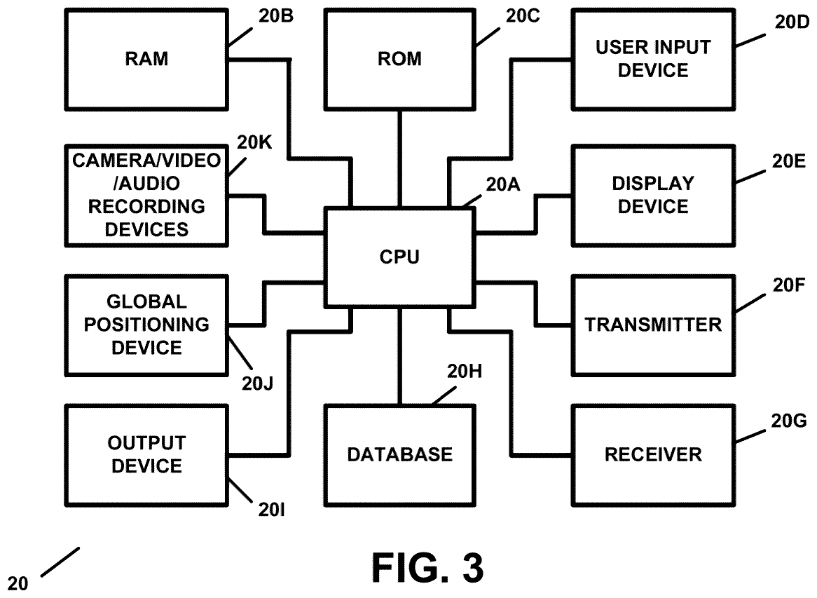

FIG. 3 illustrates a preferred embodiment of the user communication device 20 of FIG. 1, in block diagram form. In a preferred embodiment, the user communication device 20 is associated with or used by a user, individual, or account holder, who or which is utilizing the apparatus 100 to effectuate a real-time payment or a peer-to-peer payment. In another preferred embodiment, the user communication device 20 can also be associated with or used by any user or individual who or which is authorized to use effectuate a real-time payment or a peer-to-peer payment for or on behalf of the user, individual, or account holder.

In a preferred embodiment, the user communication device 20 can be a personal computer, a laptop computer, a notebook computer, a tablet, a tablet computer, a cellular telephone, a personal digital assistant, a wireless telephone, a wireless communication device, a personal communication device, a personal communications services device, a smart phone, a Smartphone, a mobile telephone, a hand-held device or computer, a palm-top device or computer, a watch, a telephone, a television, an interactive television, a digital television, a smart television or entertainment device, an internet-enabled television or entertainment device, or any other suitable device, which can be equipped to perform the functions described herein as being performed by the user communication device 20. In a preferred embodiment, the user communication device 20 can also be a cellular telephone, a personal digital assistant, or a Smartphone or smart phone which can be utilized as an electronic wallet.

In the preferred embodiment, the user communication device 20 includes a central processing unit or CPU 20A, which in the preferred embodiment, is a microprocessor. The CPU 20A may also be a microcomputer, a minicomputer, a macro-computer, and/or a mainframe computer, depending upon the application.

The user communication device 20 also includes a random access memory device(s) 20B (RAM) and a read only memory device(s) 20C (ROM), each of which is connected to the CPU 20A, and a user input device 20D, for entering data, information, and/or commands, into the user communication device 20, which includes any one or more of a keyboard, a scanner, a card reader, a barcode reader, a barcode scanner, a two-dimensional barcode reader, a two-dimensional barcode scanner, a QR code reader, a QR code scanner, an imaging device, a camera for obtaining an image of a barcode, a two-dimensional barcode, and/or a QR code, a two-dimensional image sensor, an account information data entry device, a card swiping device, a touch screen, and/or a user pointing device, such as, for example, a mouse, a touch pad, and/or an audio input device and/or a video input device, a microphone or an audio recording device, a camera or a video recording device, and/or any device, electronic and/or otherwise which can be utilized for inputting and/or entering data and/or information, of any kind or type pertinent to the operation of the apparatus 100 of the present invention, into the user communication device 20. The input device 20D can also be any other input device(s) which are or can be utilized with or in connection with any of the user communication device(s) 20 described herein as being utilized in connection with the apparatus 100 of the present invention.

The input devices 20D are also connected to or with, or linked to or with, the CPU 20A. In a preferred embodiment, the input device 20D can also include a retinal scanner, a fingerprint recognition device, a voice recognition device, or any other type or kind of biometric device which can be used for determining whether or not a user or operator of the user communication device 20 is an authorized user, individual, or person. The user communication device 20 also includes a display device 20E for displaying data and/or information to a user or operator.

The user communication device 20 also includes a transmitter(s) 20F, for transmitting signals and/or data and/or information, or a message(s), to any one or more of the central processing computer(s) 10A, the central processing computer/distributed ledger technology system 10, the counterparty communication device(s) 50, the user wallet(s) 30, the user bank/financial institution records computer(s) 40, the real-time/peer-to-peer payment provider system(s) 80, and/or any other user communication device(s) 20, described herein. The user communication device 20 can also be equipped with transmitters, receivers, network interface devices, and/or any other appropriate hardware and/or software, so as to communicate, in a bi-directional manner with, so as to transmit signals, data, information, or a message to, and/or so as to receive signals, data, information, or a message from, any of aforementioned devices, computers, or systems 20, 30, 40, 50, 60, 70, and/or 80.

The user communication device 20 also includes a receiver(s) 20G, for receiving signals and/or data and/or information, or a message(s), from any one or more of the central processing computer(s) 10A, the central processing computer/distributed ledger technology system 10, the counterparty communication device(s) 50, the user wallet(s) 30, the user bank/financial institution records computer(s) 40, the real-time/peer-to-peer payment provider system(s) 80, and/or any other user communication device(s) 20, described herein.

The user communication device 20 also includes a database(s) 20H, which is also connected to or linked with the CPU 20A, which can contain and/or include any and/or all of the data and/or information needed or desired for performing any and/or all of the functions and/or functionality described herein as being performed by the user communication device 20 and the apparatus 100 and method of the present invention, described herein or otherwise.

In a preferred embodiment, the database 20H can contain and/or include any and/or all data and/or information disclosed and described as being stored in any of the respective databases of any of the communication devices, user communication devices, and/or central processing computers, disclosed and described in U.S. patent application Ser. No. 16/509,026, filed Jul. 14, 2019, U.S. patent application Ser. No. 16/029,550, and U.S. patent application Ser. No. 17/224,838, and/or any and/or all of the other patent applications and/or provisional patent applications incorporated by reference herein, and the subject matter and teachings of the same, which are hereby incorporated by reference herein in their entirety, the subject matter and teachings of all of which are hereby incorporated by reference herein in their entirety. In a preferred embodiment, the database 20H can also contain or include any and/or all of the data, information, software, software applications, needed or desired to performing all of the herein-described functions and functionalities of the user communication device 20, the central processing computer(s) 10A, the central processing computer/distributed ledger technology system 10, and/or the apparatus 100.

In a preferred embodiment, the database 20H contains and/or includes, for each account with which the communication device 20 can be utilized, data and/or information regarding the user, individual, or account holder, of the account and any authorized user or individual who can utilize the account, including, but not limited to, his, her, or its, name, address, contact information, telephone number(s), cellular telephone number(s), wireless telephone number(s), personal communication device telephone number(s), e-mail address(es), IP address(es), text message number(s) or information, SMS message(s) or information, MMS message(s) or information, employer information, work information, emergency contact information, and/or any other contact or other information, account information, information regarding any and/or all accounts of the account holder, account numbers, account expiration dates, security codes or numbers, personal identification numbers (PINs), password(s), access code(s), social security numbers, account credit limit(s), account spending limit(s), account deductible(s), and/or any other data and/or information regarding the account holder of the account and any user(s) or individual(s) authorized to use the account. In the case of individuals, the database 20H can also contain and/or include data and/or information regarding the account holder's relatives, friends, next of kin, or other contact information or emergency contact information.

The database 20H can also contain or include, for each account, the phone number or IP address of the respective central processing computer 10A which performs transaction authorization processing for the respective account. The database 20H can also contain or include a link(s) or hyperlink(s) to each of any of the central processing computer(s) 10A associated with any of the accounts held by, owned by, or associated with, the user, individual, or account holder. The database 20H can also contain or include a link(s) or hyperlink(s) to any transaction page or pages associated with the central processing computer(s) 10A associated with any of the accounts held by, owned by, or associated with, the user, individual, or account holder. The database 20H can also contain or include a link(s) or hyperlink(s) to any counterparty communication device(s) 50 associated with any of counterparty who or with which the account can be utilized, and/or who or with which the user, individual, or account holder, or any authorized user or individual associated with a respective account can engage in a transaction(s). The database 20H can also contain or include, for each account, the times or hours of operation for each of the respective central processing computer 10A which performs transaction authorization processing for the respective account.

The database 20H can also contain or include, for each user, individual, or account holder, or for each account, contact information and/or customer service information, including, but not limited to telephone number(s), e-mail address(es), or instant messaging number or SMS messaging number, MMS messaging number, or account administrator information or customer service agent information, for the respective central processing computer 10A which performs transaction authorization processing for the respective account.

The database 20H can also contain or include a link(s) or hyperlink(s) to any user wallet 30 or user bank/financial institution records computer 40 associated with a respective user, individual, account holder, or account, or which administers a financial account associated with the respective user, individual, account holder, or account. The database 20H can also contain or include a link(s) or hyperlink(s) to any counterparty bank/financial institution records computer(s) 70 associated with a respective counterparty.

The database 20H can also contain and/or include, for each user, individual, or account holder, of the account and for any or each user or individual authorized to use the account, a description of the user, individual, or account holder, a photograph or video clip of the user, individual, or account holder, data and/or information regarding a digital voiceprint of the user, individual, or account holder, or data and/or information for verifying an identity of the user, individual, or account holder, by his or her voiceprint, data and/or information regarding a retinal scan of the user, individual, or account holder, or data and/or information for verifying an identity of the user, individual, or account holder by his or her retinal scan, data and/or information regarding a fingerprint of the user, individual, or account holder, or data and/or information for verifying an identity of the user, individual, or account holder, by his or her fingerprint, and/or any other data and/or information for identifying and identity of the user, individual, or account holder, using biometric data and/or information.

In a preferred embodiment, the database 20H can also contain and/or include, for each user, individual, or account holder, data and/or information regarding each user communication device 20 which is or can be used by the user, individual, or account holder, in utilizing the apparatus 100 and method of the present invention, including, but not limited to, data and/or information regarding an identification of each user communication device 20, a description of, or type or kind of, the communication device 20, manufacturer, model number, and/or serial number or any other identification information, of, for, or regarding, the user communication device 20, and/or the assigned telephone number, e-mail address, text messaging or SMS messaging number, MMS messaging number, and/or IP address, or any network identification information, of, for, or associated with, the user communication device 20.

The database 20H can also contain and/or include, for each user, individual, or account holder, or for each account, serviced by the user communication device 20, information about the user, individual, or account holder, and he/her/its respective account number(s), bank account numbers, payment account numbers, credit and/or account limit(s), spending limit(s), previous transactions, previous purchases, previous unauthorized transactions made on or involving the account or attempted to be made on or involving the account, previous unauthorized purchases made on or involving the account or attempted to be made on or involving the account, number of authorized and/or unauthorized transaction or purchases, account statements, historical account statements, and/or any other data and/or information needed, desired, and/or necessary, to manage and/or process an account transaction as described herein. The database 20H also contains and/or includes data and/or information regarding account statements, historical account statements, pending transactions, pending authorizations, and/or any other data and/or information regarding account activity and/or account activities.

The database 20H can also contain and/or include, for each account serviced by the user communication device 20, data and/or information about the user, individual, or account holder, and his/her/its respective account number(s), bank account numbers, payment account numbers, credit and/or account limit(s), spending limit(s), previous transactions, previous purchases, previous unauthorized transactions made on or involving the account or attempted to be made on or involving the account, previous unauthorized purchases made on or involving the account or attempted to be made on or involving the account, number of authorized and/or unauthorized transaction or purchases, account statements, historical account statements, and/or any other data and/or information needed, desired, and/or necessary, to manage and/or process an account transaction as described herein. The database 20H also contains and/or includes data and/or information regarding account statements, historical account statements, pending transactions, pending authorizations, and/or any other data and/or information regarding account activity and/or account activities.

The database 20H can also contain and/or include, for each account serviced by the user communication device 20, data and/or information regarding a user's, individual's, or account holder's, bank account numbers, payment account numbers, and/or or along with requests or instructions to receive alerts or alert messages regarding any transactions occurring on any of his, her, or its accounts.

The database 20H can also contain or include, for each user, individual, or account holder, or for each account serviced by the user communication device 20, the phone number, telephone number, url, or IP address of the respective central processing computer 10A which performs transaction processing or payment processing for the respective account. The database 20H can also contain or include a link(s) or hyperlink(s) to any central processing computer(s) 10A associated with any of the accounts held by, owned by, or associated with, each user, individual, or account holder, serviced by the apparatus 100 of the present invention and/or by the user communication device 20. The database 20H can also contain or include a link(s) or hyperlink(s) to any transaction page or web page or pages or web pages associated with the central processing computer(s) 10A associated with any of the accounts held by, owned by, or associated with, each user, individual or account holder.

The database 20H can also contain or include a link(s) or hyperlink(s) to each of the herein-described user communication devices 20, user wallet(s) 30, counterparty communication device(s) 50, user bank/financial institution records computer(s) 40, the real-time/peer-to-peer payment provider system(s) 80, and/or any other user communication device(s) 20, described herein.

The database 20H can also contain or include, for each account, the times or hours of operation of the user communication device 20 and/or for each of the respective central processing computer(s) 10A which perform transaction processing or payment processing for any of the accounts serviced by the apparatus 100 of the present invention and/or by the user communication device 20.

The database 20H can also contain or include, for each user, individual, or account holder, or for each account serviced by the user communication device 20, data and/or information regarding specific limitations and/or restrictions which may be placed on a particular account, which may be pre-selected and/or programmed by the user, individual, or account holder, and/or which may include limitations and/or restrictions on the usage of the account or any cards or account numbers associated with the account and/or which may be placed on the account by the entity which issued the account or which services the account. The limitations and/or restrictions may include the types of transactions which are allowed and/or authorized, the goods and/or services which may be purchased with the account, the vendors, stores and/or service providers, which may be authorized to accept the payment via the account, limits on the dollar amounts or other monetary amounts of transactions pertaining to each authorized vendor, seller, and/or service provider, daily spending limits, and/or the geographical area or location wherein authorized account use may be limited, and/or authorized times for account usage, such as, but not limited to, specific days, dates, time of day, time of month, year, and/or any other time of use, and/or any other limitation and/or restriction regarding amount of transaction, parties involved, geographical area, and/or times of allowed usage.

The database 20H can also contain or include, for each user, individual, or account holder, or for each account serviced by the communication device 20, data and/or information regarding transactions processed on or involving the account. The data and/or information can also include, for each of any number of transactions for each or any number of accounts, a picture, a photograph, or a video clip, of the user, individual, or account holder, or other user or individual involved in a transaction, and/or audio information, a voice message or sample, and/or a voice print, which is a digital representation of a voice message or sample, obtained from the user, individual, account holder, or other user or individual, involved in the transaction, for each of any number of transactions processed by the central processing computer 10A and/or the user communication device 20 for the account.

For example, for any given account, the central processing computer 10A and/or the user communication device 20 can store information for any number of transactions occurring on a given account. In this manner, a picture, a photograph, or a video clip, of the user, individual, or account holder, or other user or individual involved in a transaction, and/or audio information, a voice message or sample, and/or a voice print, which is a digital representation of a voice message or sample, obtained from the user, individual, or account holder, or other user or individual, involved in the transaction, can be stored for any number of transactions on a respective account, in order to perform identity verification for a user, individual, or account holder, or other user or individual, involved in a transaction.

The database 20H can also contain or include copies of any of the images, digital copies, photographs, or pictures, of any of the respective documents, authorization forms, checks, forms, receipts, request forms, or other entities, which have been, or which were, offered, presented, submitted, processed, involved, or used, in or involving, or related to, a transaction or transactions on or involving any of the accounts serviced by the user communication device 20.

It is envisioned that a user, individual, account holder, or other authorized user or individual, of a respective account can submit a photograph or picture or his or her face which can be stored in the database 20H as a “file photograph” or a “file picture” associated with the respective account. In a preferred embodiment, the “file photograph” or “file picture” can be stored as a digital file. It is also envisioned that the user, individual, account holder, or authorized user or individual, can also submit a pre-recorded voice message which can be digitized into a voice print which can be stored in the user communication device 20 as a “file voiceprint”.

In processing a transaction involving a respective account, the user communication device 20 can process a picture, a photograph, or a video clip, of the user, individual, account holder, or other individual, involved in the transaction, and/or audio information, a voice message or sample, and/or a voice print, obtained from the user, individual, account holder, or other individual, involved in the transaction using the respective “file photograph”, “file picture”, and/or “file voice print”, and/or can provide the “file photograph”, “file picture”, and/or “file voice print”, to a central processing computer 10A and/or to a counterparty communication device 50 so that the operator of same can use same in verifying that the user, individual, account holder, or other individual, is the account holder associated with the account or is an authorized user or individual associated with the account.

The database 20H can also contain or include, for each account serviced by the user communication device 20, data and/or information regarding account statements, historical statements, periodic transaction records, and/or any other data and/or information regarding past transactions and/or activities, and/or attempted transactions or attempted activities, on or involving the account, including transactions or activities that were authorized or completed and/or which were not authorized or disallowed.

The database 20H can also contain or include, for each account serviced by the user communication device 20, data and/or information regarding a barcode containing information regarding the account number associated with the respective account, data and/or information regarding a two-dimensional barcode containing information regarding the account number associated with the respective account, and/or data and/or information regarding a QR code or Quick Response code containing information regarding the account number associated with the respective account. The database 20H can also contain data and/or information and/or software processing routines for scanning, reading, and/or deciphering, data and/or information contained in a barcode, a two-dimensional barcode, and/or a QR code or Quick Response code.

The database 20H can also contain and/or include, for each user, individual, account holder, or for each account, serviced by the communication device 20, information regarding an account holder's requests or instructions to receive alerts or alert messages regarding any transactions occurring on any of his, her, or its account(s).

The database 20H can also contain or include data and/or information regarding specific limitations and/or restrictions which may be placed on a particular account, which may be pre-selected and/or programmed by the user, individual, or account holder, and which may include limitations and/or restrictions on the usage of the account or any account numbers associated with the account. The limitations and/or restrictions may include the types of transactions which are allowed and/or authorized, the goods and/or services which may be purchased with the account, the vendors, stores and/or service providers, which may be authorized to accept the payment via the account, limits on the dollar or other monetary amounts of transactions pertaining to each authorized vendor, seller, and/or service provider, daily spending limits, and/or the geographical area or location wherein authorized account use may be limited, and/or authorized times for account usage, such as, but not limited to, specific days, dates, time of day, time of month, year, and/or any other time of use, and/or any other limitation and/or restriction regarding amount of transaction, parties involved, geographical area, and/or times of allowed usage.

The database 20H can also contain or include, for each account, data and/or information regarding transactions processed on or involving the account. The data and/or information can also include, for each of any number of transactions for each or any number of accounts, a picture, a photograph, or a video clip, of the user, individual, or account holder, or other user or individual, involved in a transaction, and/or audio information, a voice message or sample, and/or a voice print, which is a digital representation of a voice message or sample, obtained from the user, individual, or account holder, or other user or individual, involved in the transaction, for each of any number of transactions processed by the user communication device 20.

For example, for any given account, the user communication device 20 can store information for any number of transactions occurring on a given account. In this manner, a picture, a photograph, or a video clip, of the user, individual, or account holder, or other user or individual, involved in a transaction, and/or audio information, a voice message or sample, and/or a voice print, which is a digital representation of a voice message or sample, obtained from the user, individual, or account holder, or other user or individual, involved in the transaction, can be stored for any number of transactions on a respective account, in order to perform identity verification for a user, individual, or account holder, or other user or individual involved in a transaction.

The database 20H can also contain or include copies of any of the images, digital copies, photographs, or pictures, of any of the respective documents, authorization forms, checks, forms, receipts, request forms, or other entities, which have been, or which were, offered, presented, submitted, processed, involved, or used, in or involving, or related to, a transaction or transactions on or involving any of the accounts serviced by the user communication device 20.

As noted herein, it is envisioned that a user, individual, or account holder, or other authorized user or individual, of a respective account can take a photograph or picture or his or her face which can be stored in the database 20H as a “file photograph” or a “file picture” associated with the respective account. In a preferred embodiment, the “file photograph” or “file picture” can be stored as a digital file in the user communication device database 20H. It is also envisioned that the user, individual, or account holder, or other authorized user or individual, can also record a voice message which can be digitized into a voice print which can be stored as a “file voiceprint”.

In processing a transaction involving a respective account, the user communication device 20 can process a picture, a photograph, or a video clip, of the user, individual, or account holder, or other authorized user or individual, involved in the transaction, and/or can process audio information, a voice message or sample, and/or a voice print, obtained from the user, individual, or account holder, or other authorized user or individual, involved in the transaction using the respective “file photograph”, “file picture”, and/or “file voice print”, and/or can provide the “file photograph”, “file picture”, and/or “file voice print”, to a counterparty communication device 50 so that the operator of same can use same in verifying that the user, individual, or account holder, or other authorized user or individual, is associated with the account or is an authorized user or individual associated with the account. In processing a transaction involving a respective account, the user communication device 20 can process a picture, a photograph, or a video clip, of the user, individual, or account holder, or other authorized user or individual, involved in the transaction, and/or can process audio information, a voice message or sample, and/or a voice print, obtained from the user, individual, or account holder, or other authorized user or individual, involved in the transaction, using the respective “file photograph”, “file picture”, and/or “file voice print”, and/or can provide the “file photograph”, “file picture”, and/or “file voice print”, to the central processing computer 10A.

The database 20H can also contain or include, for each user, individual, or account holder, or for each account, data and/or information regarding a barcode containing information regarding the account number associated with the respective user, individual, or account holder, or account, data and/or information regarding a two-dimensional barcode containing information regarding the account number associated with the respective user, individual, or account holder, or account, and/or data and/or information regarding a QR code or Quick Response code containing information regarding the account number associated with the respective user, individual, or account holder, or account. The database 20H can also contain data and/or information and/or software processing routines for scanning, reading, and/or deciphering, data and/or information contained in a barcode, a two-dimensional barcode, and/or a QR code or Quick Response code.

The database 20H can also contain or include any of the data and/or information described herein as being stored in the databases of any of the other user communication devices 20 and/or any counterparty communication devices 50 described herein. The database 20H can also contain or include any data and/or information stored in any of the user bank/financial institution records computer(s) 40 and/or counterparty bank/financial institution records computer(s) 70 described herein.

The database 20H can also contain or include any and/or all data and/or information and/or any software programs, routines, and/or software applications or “apps”, needed or desired for performing any and/or of the processing routines, functions, and/or functionality, described herein as being provided by or performed by the user communication device 20, and/or by the apparatus 100 of the present invention and/or by the central processing computer(s) 10A, the central processing computer/distributed ledger technology system 10, and/or by any counterparty communication device(s) 50, described herein.

The database 20H can also contain or include any and/or all data and/or information for or regarding the user, individual, or account holder, or other authorized user or individual for facilitating using the user communication device 20 as an electronic wallet or a user wallet 30. In this regard, the database 20H can contain or include an electronic version of the respective user's, individual's, or account holder's, driver's license, identification information, social security card, any professional license(s), vehicle registration(s), automobile insurance card(s), passport(s), home insurance policy, malpractice insurance policy, health insurance policy, life insurance policy, disability insurance policy, and/or an electronic version or any account card(s) associated with any of the user's, individual's, or account holder's, various accounts, which can be any one or more, or any combination, of the various accounts described herein.

The database 20H can also contain or include any and/or all data and/or information and/or any software programs, routines, and/or software applications or “apps”, needed or desired for performing any and/or of the processing routines, functions, and/or functionality, described herein as being performed by or provided by the user communication device 20, any other communication devices 20 associated with the user, individual, or account holder, and/or any counterparty communication devices 50, with such data and/or information and/or software applications or “apps”, being downloadable to the user communication device 20 if and when needed or desired.

In a preferred embodiment, the database 20H can also contain or include any and/or all data and/or information needed, desired, or utilized, by the apparatus 100, or by the central processing computer(s) 10A and/or the central processing computer/distributed ledger technology system 10, and/or by any other user communication device(s) 20 and/or counterparty communication device(s) 50, described herein for or in performing any and/or all of the processing routines, operations, functions, and/or functionality, described herein as being performed by the user communication device 20 and/or the apparatus 100 and method of the present invention.

The communication device 20 also includes an output device 20I, which is also connected to the CPU 20A, for outputting any data and/or information, described herein. In the preferred embodiment, the output device 20I can be a printer, a display, a transmitter, a modem, and/or any other device which can be used to output data or information.

The communication device 20 can also be equipped with a global positioning device 20J which can be connected to the CPU 20A and which can be utilized to calculate, determine, or ascertain, the position or location of the communication device 20.

The communication device 20 can also include a video and/or audio recording device 20K which, in a preferred embodiment, can include a camera, a video recoding device, a microphone, and/or an audio recording device. The video and/or audio recording device 20K can be utilized to take a picture, record video, record a video clip, record sound, record audio, or record an audio clip, of a user of the communication device 20 and/or to record any picture, a sound or voice, video information, or audio information at the communication device 20.

In a preferred embodiment, the user communication device 20 can serve as a transaction authorization processing device which can communicate, in bi-directional manner, with any central processing computer 10A and/or counterparty communication device 50 which can perform payment processing and/or transaction processing for any account described herein. In this regard, in a preferred embodiment, the user communication device 20 can also be equipped with the needed hardware and/or software to function as a point of sale (POS) transaction device which can communicate, in a bi-directional manner, with any central processing computer 10A and/or any transaction processing computer or any transaction authorization processing computer.

In another preferred embodiment, the user communication device 20 can include, and/or can be utilized in conjunction with, any of the herein-described user input devices 20D which can be separate and apart from the user communication device 20. In such an embodiment, such user input device(s) 20D can be wirelessly linked to the CPU 20A or to the communication device 20 with, using, or via, a Wi-Fi connection or Wi-Fi equipment, or a Bluetooth connection or Bluetooth equipment, or any combination of same.

In another preferred embodiment, the user communication device 20 can include, and/or can be utilized in conjunction with, any of the herein-described output devices 20I which can be separate and apart from the user communication device 20. In such an embodiment, such output device(s) 20I can be wirelessly linked to the CPU 20A or to the user communication device 20 with, using, or via, a Wi-Fi connection or Wi-Fi equipment, or a Bluetooth connection or Bluetooth equipment, or any combination of same.

In another preferred embodiment, the user communication device 20 can include, and/or can be utilized in conjunction with, any of the herein-described camera/video/audio equipment systems 20K which can be separate and apart from the user communication device 20. In such an embodiment, such camera/video/audio equipment system(s) 20K can be wirelessly linked to the CPU 20A or to the user communication device 20 with, using, or via, a Wi-Fi connection or Wi-Fi equipment, or a Bluetooth connection or Bluetooth equipment, or any combination of same.

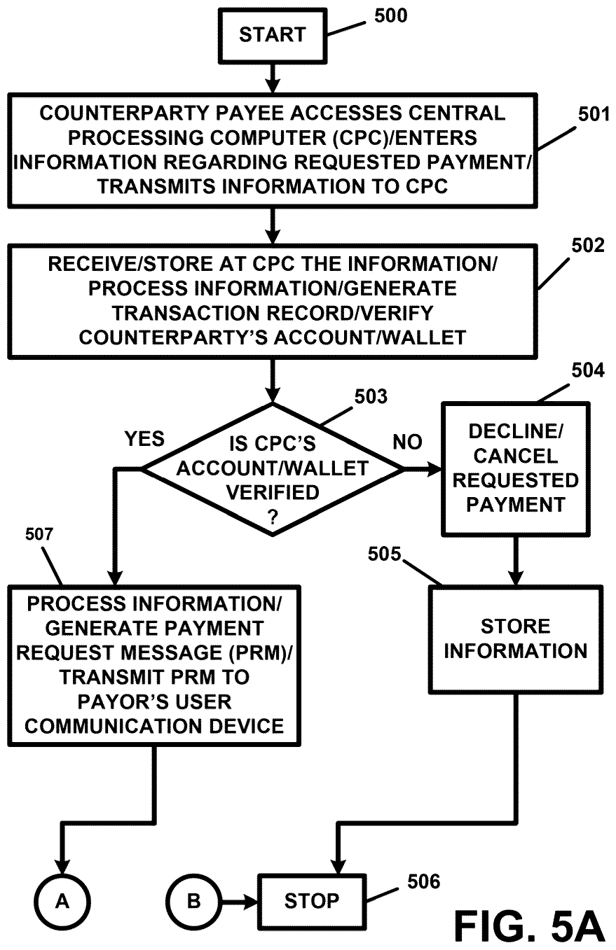

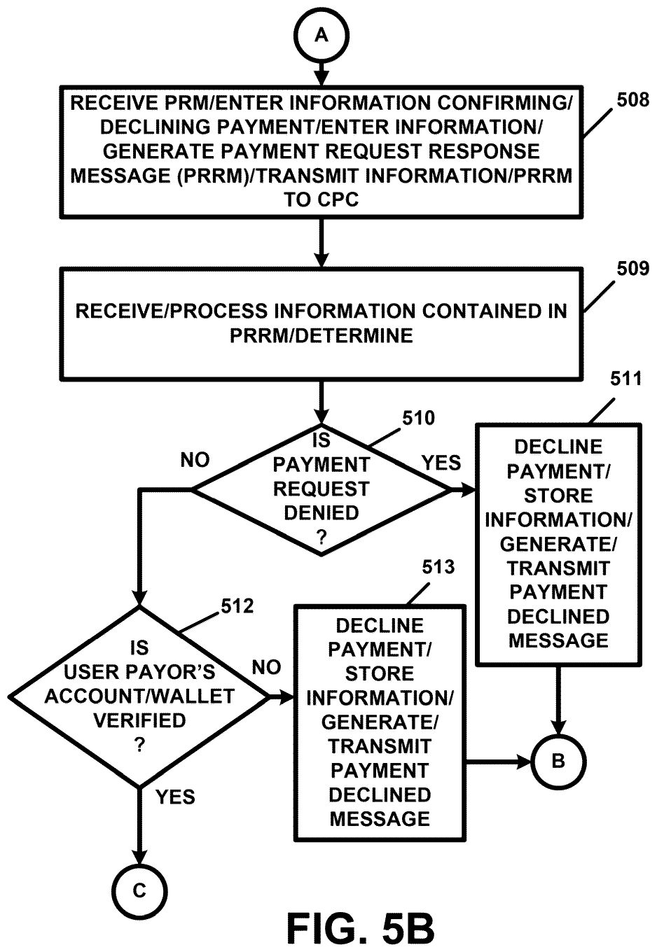

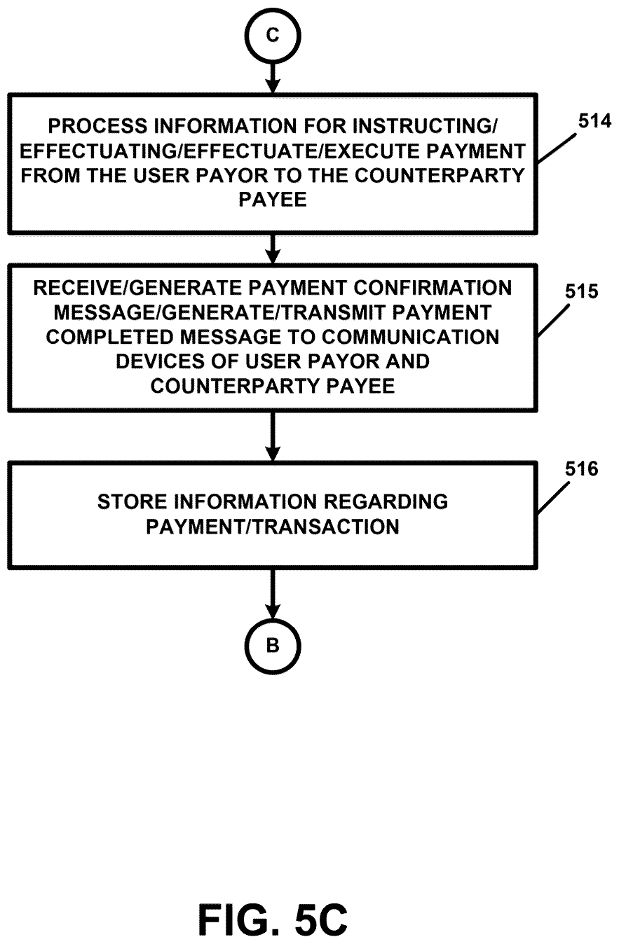

FIG. 5 illustrates a preferred embodiment of the counterparty communication device 50 of FIG. 1, in block diagram form. In a preferred embodiment, the counterparty communication device 50 is associated with or used by a counterparty user (or “counterparty”) who or which is utilizing the apparatus 100 to receive a real-time payment or a peer-to-peer payment. In another preferred embodiment, the counterparty communication device 50 can also be associated with or used by any individual or entity who or which is authorized to receive a real-time payment or a peer-to-peer payment for or on behalf of the counterparty.

In a preferred embodiment, the counterparty communication device 50 can be a personal computer, a laptop computer, a notebook computer, a tablet, a tablet computer, a cellular telephone, a personal digital assistant, a wireless telephone, a wireless communication device, a personal communication device, a personal communications services device, a smart phone, a Smartphone, a mobile telephone, a hand-held device or computer, a palm-top device or computer, a watch, a telephone, a television, an interactive television, a digital television, a smart television or entertainment device, an internet-enabled television or entertainment device, or any other suitable device, which can be equipped to perform the functions described herein as being performed by the counterparty communication device 50. In a preferred embodiment, the counterparty communication device 50 can also be a cellular telephone, a personal digital assistant, or a Smartphone or smart phone which can be utilized as an electronic wallet.

In the preferred embodiment, the counterparty communication device 50 includes a central processing unit or CPU 50A, which in the preferred embodiment, is a microprocessor. The CPU 50A may also be a microcomputer, a minicomputer, a macro-computer, and/or a mainframe computer, depending upon the application.

The counterparty communication device 50 also includes a random access memory device(s) 50B (RAM) and a read only memory device(s) 50C (ROM), each of which is connected to the CPU 50A, and a user input device 50D, for entering data, information, and/or commands, into the counterparty communication device 50, which includes any one or more of a keyboard, a scanner, a card reader, a barcode reader, a barcode scanner, a two-dimensional barcode reader, a two-dimensional barcode scanner, a QR code reader, a QR code scanner, an imaging device, a camera for obtaining an image of a barcode, a two-dimensional barcode, and/or a QR code, a two-dimensional image sensor, an account information data entry device, a card swiping device, a touch screen, and/or a user pointing device, such as, for example, a mouse, a touch pad, and/or an audio input device and/or a video input device, a microphone or an audio recording device, a camera or a video recording device, and/or any device, electronic and/or otherwise which can be utilized for inputting and/or entering data and/or information, of any kind or type pertinent to the operation of the apparatus 100 of the present invention, into the counterparty communication device 50. The input device 50D can also be any other input device(s) which are or can be utilized with or in connection with any of the counterparty communication device(s) 50 described herein as being utilized in connection with the apparatus 100 of the present invention.

The input devices 50D are also connected to or with, or linked to or with, the CPU 50A. In a preferred embodiment, the input device 50D can also include a retinal scanner, a fingerprint recognition device, a voice recognition device, or any other type or kind of biometric device which can be used for determining whether or not a user or operator of the counterparty communication device 50 is an authorized user, individual, or person. The counterparty communication device 50 also includes a display device 50E for displaying data and/or information to a user or operator.

The counterparty communication device 50 also includes a transmitter(s) 50F, for transmitting signals and/or data and/or information, or a message(s), to any one or more of the central processing computer(s) 10A, the central processing computer/distributed ledger technology system 10, the user communication device(s) 20, the counterparty wallet(s) 60, the counterparty bank/financial records computer(s) 70, the real-time/peer-to-peer payment provider system(s) 80, and/or any other counterparty communication device(s) 50, described herein. The counterparty communication device 50 can also be equipped with transmitters, receivers, network interface devices, and/or any other appropriate hardware and/or software, so as to communicate, in a bi-directional manner with, so as to transmit signals, data, information, or a message to, and/or so as to receive signals, data, information, or a message from, any of aforementioned devices, computers, or systems 20, 30, 40, 50, 60, 70, and/or 80.

The counterparty communication device 50 also includes a receiver(s) 50G, for receiving signals and/or data and/or information, or a message(s), from any one or more of the central processing computer(s) 10A, the central processing computer/distributed ledger technology system 10, the user communication device(s) 20, the counterparty wallet(s) 60, the counterparty bank/financial records computer(s) 70, the real-time/peer-to-peer payment provider system(s) 80, and/or any other counterparty communication device(s) 50, described herein.

The counterparty communication device 50 also includes a database(s) 50H, which is also connected to or linked with the CPU 50A, which can contain and/or include any and/or all of the data and/or information needed or desired for performing any and/or all of the functions and/or functionality described herein as being performed by the counterparty communication device 50 and the apparatus 100 and method of the present invention, described herein or otherwise.

In a preferred embodiment, the database 50H can contain and/or include any and/or all data and/or information disclosed and described as being stored in any of the respective databases of any of the communication devices, user communication devices, counterparty communication devices, and/or central processing computers, disclosed and described in U.S. patent application Ser. No. 16/509,026, filed Jul. 14, 2019, U.S. patent application Ser. No. 16/029,550, and U.S. patent application Ser. No. 17/224,838, and/or any and/or all of the other patent applications and/or provisional patent applications incorporated by reference herein, and the subject matter and teachings of the same, the subject matter and teachings of all of which are hereby incorporated by reference herein in their entirety.

In a preferred embodiment, the database 50H can also contain or include any and/or all of the data, information, software, software applications, needed or desired to performing all of the herein-described functions and functionalities of the counterparty communication device 50, the central processing computer(s) 10A, the central processing computer/distributed ledger technology system 10, and/or the apparatus 100.

In a preferred embodiment, the database 50H contains and/or includes, for each account with which the counterparty communication device 50 can be utilized, data and/or information regarding the user, individual, or account holder, of the account and any authorized user or individual who can utilize the account, including, but not limited to, his, her, or its, name, address, contact information, telephone number(s), cellular telephone number(s), wireless telephone number(s), personal communication device telephone number(s), e-mail address(es), IP address(es), text message number(s) or information, SMS message(s) or information, MMS message(s) or information, employer information, work information, emergency contact information, and/or any other contact or other information, account information, information regarding any and/or all accounts of the account holder, account numbers, account expiration dates, security codes or numbers, personal identification numbers (PINs), password(s), access code(s), social security numbers, account credit limit(s), account spending limit(s), account deductible(s), and/or any other data and/or information regarding the account holder of the account and any user(s) or individual(s) authorized to use the account. In the case of individuals, the database 50H can also contain and/or include data and/or information regarding the account holder's relatives, friends, next of kin, or other contact information or emergency contact information.

The database 50H can also contain or include, for each account, the phone number or IP address of the respective central processing computer 10A which performs transaction authorization processing for the respective account. The database 50H can also contain or include a link(s) or hyperlink(s) to each of any of the central processing computer(s) 10A associated with any of the accounts held by, owned by, or associated with, the counterparty. The database 50H can also contain or include a link(s) or hyperlink(s) to any transaction page or pages associated with the central processing computer(s) 10A associated with any of the accounts held by, owned by, or associated with, the counterparty. The database 50H can also contain or include a link(s) or hyperlink(s) to any counterparty communication device(s) 50 associated with any counterparty who or with which the account can be utilized, and/or who or with which the counterparty, or any authorized user or individual associated with a respective account can engage in a transaction(s). The database 50H can also contain or include, for each account, the times or hours of operation for each of the respective central processing computer 10A which performs transaction authorization processing for the respective account.

The database 50H can also contain or include, for each counterparty, user, individual, or account holder, or for each account, contact information and/or customer service information, including, but not limited to telephone number(s), e-mail address(es), or instant messaging number or SMS messaging number, MMS messaging number, or account administrator information or customer service agent information, for the respective central processing computer 10A which performs transaction authorization processing for the respective account.

The database 50H can also contain or include a link(s) or hyperlink(s) to any counterparty wallet 60 or user bank/financial institution records computer 70 associated with the counterparty, or which administers a financial account associated with the counterparty or account. The database 50H can also contain or include a link(s) or hyperlink(s) to any user bank/financial institution records computer(s) 40 associated with a respective user and/or any other counterparty bank/financial institution records computer(s) 70 associated with the counterparty or any other counterparty.

The database 50H can also contain and/or include, for each counterparty or for each user, individual, or account holder, of the account and for any or each user or individual authorized to use the account, a description of the counterparty or the user, individual, or account holder, a photograph or video clip of the counterparty or the user, individual, or account holder, data and/or information regarding a digital voiceprint of the counterparty or the user, individual, or account holder, or data and/or information for verifying an identity of the counterparty or the user, individual, or account holder, by his or her voiceprint, data and/or information regarding a retinal scan of the counterparty or the user, individual, or account holder, or data and/or information for verifying an identity of the counterparty or the user, individual, or account holder by his or her retinal scan, data and/or information regarding a fingerprint of the counterparty or the user, individual, or account holder, or data and/or information for verifying an identity of the counterparty or the user, individual, or account holder, by his or her fingerprint, and/or any other data and/or information for identifying and identity of the counterparty or the user, individual, or account holder, using biometric data and/or information.

In a preferred embodiment, the database 50H can also contain and/or include, for each counterparty for each user, individual, or account holder, data and/or information regarding each counterparty communication device 50 which is or can be used by the counterparty or the user, individual, or account holder, in utilizing the apparatus 100 and method of the present invention, including, but not limited to, data and/or information regarding an identification of the counterparty or each counterparty communication device 50, a description of, or type or kind of, the counterparty communication device 50, the manufacturer, model number, and/or serial number or any other identification information, of, for, or regarding, the counterparty communication device 50, and/or the assigned telephone number, e-mail address, text messaging or SMS messaging number, MMS messaging number, and/or IP address, or any network identification information, of, for, or associated with, the counterparty communication device 50.

The database 50H can also contain and/or include, for each counterparty or for user, individual, or account holder, or for each account, serviced by the counterparty communication device 50, information about the counterparty or the user, individual, or account holder, and he/her/its respective account number(s), bank account numbers, payment account numbers, credit and/or account limit(s), spending limit(s), previous transactions, previous purchases, previous unauthorized transactions made on or involving the account or attempted to be made on or involving the account, previous unauthorized purchases made on or involving the account or attempted to be made on or involving the account, number of authorized and/or unauthorized transaction or purchases, account statements, historical account statements, and/or any other data and/or information needed, desired, and/or necessary, to manage and/or process an account transaction as described herein. The database 50H also contains and/or includes data and/or information regarding account statements, historical account statements, pending transactions, pending authorizations, and/or any other data and/or information regarding account activity and/or account activities.

The database 50H can also contain and/or include, for each account serviced by the counterparty communication device 50, data and/or information about the counterparty or the user, individual, or account holder, and his/her/its respective account number(s), bank account numbers, payment account numbers, credit and/or account limit(s), spending limit(s), previous transactions, previous purchases, previous unauthorized transactions made on or involving the account or attempted to be made on or involving the account, previous unauthorized purchases made on or involving the account or attempted to be made on or involving the account, number of authorized and/or unauthorized transaction or purchases, account statements, historical account statements, and/or any other data and/or information needed, desired, and/or necessary, to manage and/or process an account transaction as described herein. The database 50H also contains and/or includes data and/or information regarding account statements, historical account statements, pending transactions, pending authorizations, and/or any other data and/or information regarding account activity and/or account activities.

The database 50H can also contain and/or include, for each account serviced by the counterparty communication device 50, data and/or information regarding a counterparty's or a user's, individual's, or account holder's, bank account numbers, payment account numbers, and/or or along with requests or instructions to receive alerts or alert messages regarding any transactions occurring on any of his, her, or its accounts.

The database 50H can also contain or include, for each counterparty or for each user, individual, or account holder, or for each account serviced by the counterparty communication device 50, the phone number, telephone number, url, or IP address of the respective central processing computer 10A which performs transaction processing or payment processing for the respective account. The database 50H can also contain or include a link(s) or hyperlink(s) to any central processing computer(s) 10A associated with any of the accounts held by, owned by, or associated with, each counterparty serviced by the apparatus 100 of the present invention and/or by the counterparty communication device 50. The database 50H can also contain or include a link(s) or hyperlink(s) to any transaction page or web page or pages or web pages associated with the central processing computer(s) 10A associated with any of the accounts held by, owned by, or associated with, each counterparty or account holder.

The database 50H can also contain or include a link(s) or hyperlink(s) to each of the herein-described counterparty communication devices 50, counterparty wallet(s) 60, user communication device(s) 20, counterparty bank/financial institution records computer(s) 70, the real-time/peer-to-peer payment provider system(s) 80, and/or any other counterparty communication device(s) 50, described herein.

The database 50H can also contain or include, for each account, the times or hours of operation of the counterparty communication device 50 and/or for each of the respective central processing computer(s) 10A which perform transaction processing or payment processing for any of the accounts serviced by the apparatus 100 of the present invention and/or by the counterparty communication device 50.

The database 50H can also contain or include, for each counterparty or for each user, individual, or account holder, or for each account serviced by the counterparty communication device 50, data and/or information regarding specific limitations and/or restrictions which may be placed on a particular account, which may be pre-selected and/or programmed by the counterparty or by each user, individual, or account holder, and/or which may include limitations and/or restrictions on the usage of the account or any cards or account numbers associated with the account and/or which may be placed on the account by the entity which issued the account or which services the account. The limitations and/or restrictions may include the types of transactions which are allowed and/or authorized, the goods and/or services which may be purchased with the account, the vendors, stores and/or service providers, which may be authorized to accept the payment via the account, limits on the dollar amounts or other monetary amounts of transactions pertaining to each authorized vendor, seller, and/or service provider, daily spending limits, and/or the geographical area or location wherein authorized account use may be limited, and/or authorized times for account usage, such as, but not limited to, specific days, dates, time of day, time of month, year, and/or any other time of use, and/or any other limitation and/or restriction regarding amount of transaction, parties involved, geographical area, and/or times of allowed usage.

The database 50H can also contain or include, for each counterparty or for each user, individual, or account holder, or for each account serviced by the counterparty communication device 50, data and/or information regarding transactions processed on or involving the account. The data and/or information can also include, for each of any number of transactions for each or any number of accounts, a picture, a photograph, or a video clip, of the counterparty or user, individual, or account holder, or other user or individual involved in a transaction, and/or audio information, a voice message or sample, and/or a voice print, which is a digital representation of a voice message or sample, obtained from the counterparty or from the user, individual, account holder, or other user or individual, involved in the transaction, for each of any number of transactions processed by the central processing computer 10A and/or the counterparty communication device 50 for the account.

For example, for any given account, the central processing computer 10A and/or the counterparty communication device 50 can store information for any number of transactions occurring on a given account. In this manner, a picture, a photograph, or a video clip, of the counterparty or the user, individual, or account holder, or other user or individual involved in a transaction, and/or audio information, a voice message or sample, and/or a voice print, which is a digital representation of a voice message or sample, obtained from the counterparty or from the user, individual, or account holder, or other user or individual, involved in the transaction, can be stored for any number of transactions on a respective account, in order to perform identity verification for a counterparty or for a user, individual, or account holder, or other user or individual, involved in a transaction.

The database 50H can also contain or include copies of any of the images, digital copies, photographs, or pictures, of any of the respective documents, authorization forms, checks, forms, receipts, request forms, or other entities, which have been, or which were, offered, presented, submitted, processed, involved, or used, in or involving, or related to, a transaction or transactions on or involving any of the accounts serviced by the counterparty communication device 50.

It is envisioned that a counterparty or other user, individual, account holder, or other authorized user or individual, of a respective account can submit a photograph or picture or his or her face which can be stored in the database 50H as a “file photograph” or a “file picture” associated with the respective account. In a preferred embodiment, the “file photograph” or “file picture” can be stored as a digital file. It is also envisioned that the counterparty or other user, individual, account holder, or authorized user or individual, can also submit a pre-recorded voice message which can be digitized into a voice print which can be stored in the counterparty communication device 50 as a “file voiceprint”.

In processing a transaction involving a respective account, the counterparty communication device 50 can process a picture, a photograph, or a video clip, of the user, individual, account holder, or other individual, involved in the transaction, and/or audio information, a voice message or sample, and/or a voice print, obtained from the user, individual, account holder, or other individual, involved in the transaction using the respective “file photograph”, “file picture”, and/or “file voice print”, and/or can provide the “file photograph”, “file picture”, and/or “file voice print”, to a central processing computer 10A and/or to a user communication device 20 so that the operator of same can use same in verifying that the counterparty is the account holder associated with the account or is an authorized user or individual associated with the account.

The database 50H can also contain or include, for each account serviced by the counterparty communication device 50, data and/or information regarding account statements, historical statements, periodic transaction records, and/or any other data and/or information regarding past transactions and/or activities, and/or attempted transactions or attempted activities, on or involving the account, including transactions or activities that were authorized or completed and/or which were not authorized or disallowed.

The database 50H can also contain or include, for each account serviced by the counterparty communication device 50, data and/or information regarding a barcode containing information regarding the account number associated with the respective account, data and/or information regarding a two-dimensional barcode containing information regarding the account number associated with the respective account, and/or data and/or information regarding a QR code or Quick Response code containing information regarding the account number associated with the respective account. The database 50H can also contain data and/or information and/or software processing routines for scanning, reading, and/or deciphering, data and/or information contained in a barcode, a two-dimensional barcode, and/or a QR code or Quick Response code.

The database 50H can also contain and/or include, for each counterparty or for each user, individual, account holder, or for each account, serviced by the counterparty communication device 50, information regarding an account holder's requests or instructions to receive alerts or alert messages regarding any transactions occurring on any of his, her, or its account(s).

The database 50H can also contain or include data and/or information regarding specific limitations and/or restrictions which may be placed on a particular account, which may be pre-selected and/or programmed by the counterparty or by a user, individual, or account holder, and which may include limitations and/or restrictions on the usage of the account or any account numbers associated with the account. The limitations and/or restrictions may include the types of transactions which are allowed and/or authorized, the goods and/or services which may be purchased with the account, the vendors, stores and/or service providers, which may be authorized to accept the payment via the account, limits on the dollar or other monetary amounts of transactions pertaining to each authorized vendor, seller, and/or service provider, daily spending limits, and/or the geographical area or location wherein authorized account use may be limited, and/or authorized times for account usage, such as, but not limited to, specific days, dates, time of day, time of month, year, and/or any other time of use, and/or any other limitation and/or restriction regarding amount of transaction, parties involved, geographical area, and/or times of allowed usage.

The database 50H can also contain or include, for each account, data and/or information regarding transactions processed on or involving the account. The data and/or information can also include, for each of any number of transactions for each or any number of accounts, a picture, a photograph, or a video clip, of the counterparty and/or a user, individual, or account holder, or other user or individual, involved in a transaction, and/or audio information, a voice message or sample, and/or a voice print, which is a digital representation of a voice message or sample, obtained from the counterparty or from the user, individual, or account holder, or other user or individual, involved in the transaction, for each of any number of transactions processed by the counterparty communication device 50.

For example, for any given account, the counterparty communication device 50 can store information for any number of transactions occurring on a given account. In this manner, a picture, a photograph, or a video clip, of the counterparty of any other user, individual, or account holder, involved in a transaction, and/or audio information, a voice message or sample, and/or a voice print, which is a digital representation of a voice message or sample, obtained from the counterparty or from the user, individual, or account holder, involved in the transaction, can be stored for any number of transactions on a respective account, in order to perform identity verification for a counterparty or for a user, individual, or account holder, involved in a transaction.

The database 50H can also contain or include copies of any of the images, digital copies, photographs, or pictures, of any of the respective documents, authorization forms, checks, forms, receipts, request forms, or other entities, which have been, or which were, offered, presented, submitted, processed, involved, or used, in or involving, or related to, a transaction or transactions on or involving any of the accounts serviced by the counterparty communication device 50.

As noted herein, it is envisioned that a counterparty or any other user, individual, or account holder, or other authorized user or individual, of a respective account can take a photograph or picture or his or her face which can be stored in the database 50H as a “file photograph” or a “file picture” associated with the respective account. In a preferred embodiment, the “file photograph” or “file picture” can be stored as a digital file in the counterparty communication device database 50H. It is also envisioned that the counterparty or the user, individual, or account holder, or other authorized user or individual, can also record a voice message which can be digitized into a voice print which can be stored as a “file voiceprint”.

In processing a transaction involving a respective account, the counterparty communication device 50 can process a picture, a photograph, or a video clip, of the counterparty or other user, individual, or account holder, or other authorized user or individual, involved in the transaction, and/or can process audio information, a voice message or sample, and/or a voice print, obtained from the counterparty or from the user, individual, or account holder, or other authorized user or individual, involved in the transaction using the respective “file photograph”, “file picture”, and/or “file voice print”, and/or can provide the “file photograph”, “file picture”, and/or “file voice print”, to a user communication device 20 so that the operator of same can use same in verifying that the counterparty or other user, individual, or account holder, or other authorized user or individual, is associated with the account or is an authorized user or individual associated with the account.

In processing a transaction involving a respective account, the counterparty communication device 50 can process a picture, a photograph, or a video clip, of the counterpary or other user, individual, or account holder, or other authorized user or individual, involved in the transaction, and/or can process audio information, a voice message or sample, and/or a voice print, obtained from the counterparty, or from the user, individual, or account holder, or other authorized user or individual, involved in the transaction, using the respective “file photograph”, “file picture”, and/or “file voice print”, and/or can provide the “file photograph”, “file picture”, and/or “file voice print”, to the central processing computer 10A.

The database 50H can also contain or include, for each counterparty or for each user, individual, or account holder, or for each account, data and/or information regarding a barcode containing information regarding the account number associated with the respective counterparty or other user, individual, or account holder, or account, data and/or information regarding a two-dimensional barcode containing information regarding the account number associated with the respective counterparty or other user, individual, or account holder, or account, and/or data and/or information regarding a QR code or Quick Response code containing information regarding the account number associated with the respective counterparty or other user, individual, or account holder, or account. The database 50H can also contain data and/or information and/or software processing routines for scanning, reading, and/or deciphering, data and/or information contained in a barcode, a two-dimensional barcode, and/or a QR code or Quick Response code.

The database 50H can also contain or include any of the data and/or information described herein as being stored in the databases of any of the user communication devices 20 and/or any other counterparty communication devices 50 described herein. The database 50H can also contain or include any data and/or information stored in any of the counterparty bank/financial institution records computer(s) 70 and/or user bank/financial institution records computer(s) 40 described herein.

The database 50H can also contain or include any and/or all data and/or information and/or any software programs, routines, and/or software applications or “apps”, needed or desired for performing any and/or of the processing routines, functions, and/or functionality, described herein as being provided by or performed by the counterparty communication device 50, and/or by the apparatus 100 of the present invention and/or by the central processing computer(s) 10A, the central processing computer/distributed ledger technology system 10, and/or by any user communication device(s) 20, described herein.

The database 50H can also contain or include any and/or all data and/or information for or regarding the counterparty, or other authorized user or individual for facilitating using the counterparty communication device 50 as an electronic wallet or a counterparty wallet 60. In this regard, the database 50H can contain or include an electronic version of the respective counterparty's driver's license, identification information, social security card, any professional license(s), vehicle registration(s), automobile insurance card(s), passport(s), home insurance policy, malpractice insurance policy, health insurance policy, life insurance policy, disability insurance policy, and/or an electronic version or any account card(s) associated with any of the counterparty's various accounts, which can be any one or more, or any combination, of the various accounts described herein.

The database 50H can also contain or include any and/or all data and/or information and/or any software programs, routines, and/or software applications or “apps”, needed or desired for performing any and/or of the processing routines, functions, and/or functionality, described herein as being performed by or provided by the counterparty communication device 50, any other counterparty communication devices 50 associated with the counterparty, and/or any user communication devices 20, with such data and/or information and/or software applications or “apps”, being downloadable to the counterparty communication device 50 if and when needed or desired.

In a preferred embodiment, the database 50H can also contain or include any and/or all data and/or information needed, desired, or utilized, by the apparatus 100, or by the central processing computer(s) 10A and/or the central processing computer/distributed ledger technology system 10, and/or by any other counterparty communication device(s) 50 and/or user communication device(s) 20, described herein for or in performing any and/or all of the processing routines, operations, functions, and/or functionality, described herein as being performed by the counterparty communication device 50 and/or the apparatus 100 and method of the present invention.

The counterparty communication device 50 also includes an output device 50I, which is also connected to the CPU 50A, for outputting any data and/or information, described herein. In the preferred embodiment, the output device 50I can be a printer, a display, a transmitter, a modem, and/or any other device which can be used to output data or information.

The counterparty communication device 50 can also be equipped with a global positioning device 50J which can be connected to the CPU 50A and which can be utilized to calculate, determine, or ascertain, the position or location of the counterparty communication device 50.

The counterparty communication device 50 can also include a video and/or audio recording device 50K which, in a preferred embodiment, can include a camera, a video recoding device, a microphone, and/or an audio recording device. The video and/or audio recording device 50K can be utilized to take a picture, record video, record a video clip, record sound, record audio, or record an audio clip, of a user of the counterparty communication device 50 and/or to record any picture, a sound or voice, video information, or audio information at the counterparty communication device 50.

In a preferred embodiment, the counterparty communication device 50 can serve as a transaction authorization processing device which can communicate, in bi-directional manner, with any central processing computer 10A and/or user communication device 20 which can perform payment processing and/or transaction processing for any account described herein. In this regard, in a preferred embodiment, the counterparty communication device 50 can also be equipped with the needed hardware and/or software to function as a point of sale (POS) transaction device which can communicate, in a bi-directional manner, with any central processing computer 10A and/or any transaction processing computer or any transaction authorization processing computer.

In another preferred embodiment, the counterparty communication device 50 can include, and/or can be utilized in conjunction with, any of the herein-described user input devices 50D which can be separate and apart from the counterparty communication device 50. In such an embodiment, such user input device(s) 50D can be wirelessly linked to the CPU 50A or to the counterparty communication device 50 with, using, or via, a Wi-Fi connection or Wi-Fi equipment, or a Bluetooth connection or Bluetooth equipment, or any combination of same.

In another preferred embodiment, the counterparty communication device 50 can include, and/or can be utilized in conjunction with, any of the herein-described output devices 50I which can be separate and apart from the counterparty communication device 50. In such an embodiment, such output device(s) 50I can be wirelessly linked to the CPU 50A or to the counterparty communication device 50 with, using, or via, a Wi-Fi connection or Wi-Fi equipment, or a Bluetooth connection or Bluetooth equipment, or any combination of same.

In another preferred embodiment, the counterparty communication device 50 can include, and/or can be utilized in conjunction with, any of the herein-described camera/video/audio equipment systems 50K which can be separate and apart from the counterparty communication device 50. In such an embodiment, such camera/video/audio equipment system(s) 50K can be wirelessly linked to the CPU 50A or to the counterparty communication device 50 with, using, or via, a Wi-Fi connection or Wi-Fi equipment, or a Bluetooth connection or Bluetooth equipment, or any combination of same.

In a preferred embodiment, the apparatus 100 and method of the present invention can be utilized to in order to provide and/or to process real-time payments and/or real-time payment transactions and/or peer-to-peer payments and/or peer-to-peer payment transactions, and to provide for the security of the same. In a preferred embodiment, the apparatus 100 and method of the present invention can be utilized to in order to provide and/or to process real-time payments and/or real-time payment transactions and/or peer-to-peer payments and/or peer-to-peer payment transactions, and to provide for the security of the same in transactions involving any two or more individuals and/or entities.

In a preferred embodiment, the apparatus 100 and method of the present invention can be utilized to in order to provide and/or to process real-time payments and/or real-time payment transactions and/or peer-to-peer payments and/or peer-to-peer payment transactions, and to provide for the security of the same in transactions involving any number or types of accounts serviced by any bank, financial institution, or financial intermediary, or for any number or types of accounts serviced by any number of banks, financial institutions, or financial intermediaries.

In a preferred embodiment, the apparatus 100 and method of the present invention can also be utilized to in order to provide and/or to process real-time payments and/or real-time payment transactions and/or peer-to-peer payments and/or peer-to-peer payment transactions, and provide for the security of the same in transactions involving any number, or any type or kind, of an any, or any combination of, credit accounts, credit card accounts, debit accounts, debit card accounts, charge accounts, charge card accounts, bank accounts, checking accounts, savings accounts, electronic money accounts, gambling accounts, gaming accounts, sports betting accounts, lottery gaming accounts, brokerage accounts, healthcare insurance accounts, and/or any of the other accounts described herein, such as, but not limited to, pension accounts, individual retirement accounts (IRAs), or self-employed pension (SEP) accounts, “smart” card accounts, currency card accounts, healthcare accounts, Medicare accounts, Medicaid accounts, employee benefits accounts, cafeteria accounts, or spending accounts, subscription accounts for any goods, products, or services, or insurance accounts, healthcare spending accounts, life insurance accounts, or disability insurance accounts, or tuition accounts, pharmacy accounts, credit report accounts, cable television accounts, digital television accounts, or satellite television accounts, social security accounts, liability insurance accounts, or lease insurance accounts, ticket accounts, telephone calling card accounts, utility accounts, electrical utility accounts, gas utility accounts, or fuel oil utility accounts, accounts monitoring use of official seals, accounts monitoring use of private, individual, and/or organizational, seals or access codes, security access accounts, computer access code accounts, facility access accounts, or facility security accounts, financial accounts, or electronic cash accounts, communication accounts, telephone accounts, wireless communication device accounts, non-wireless communication device accounts, cellular communication device accounts, cellular telephone accounts, Internet accounts, or Internet service provider accounts, electronic signature accounts, e-mail accounts, membership accounts, club membership accounts, entertainment membership accounts, entertainment tickets accounts, sports tickets accounts, theatre tickets accounts, concert or opera tickets accounts, consumer or purchaser memberships accounts, sports club membership accounts, or health club membership accounts, merchant credit accounts for customers, merchant accounts, association membership accounts, professional association membership accounts, or trade association membership accounts, or any other accounts described herein, as well as any cards, devices, and/or other entities, which can be used with or which can be associated with any of the herein-described accounts.

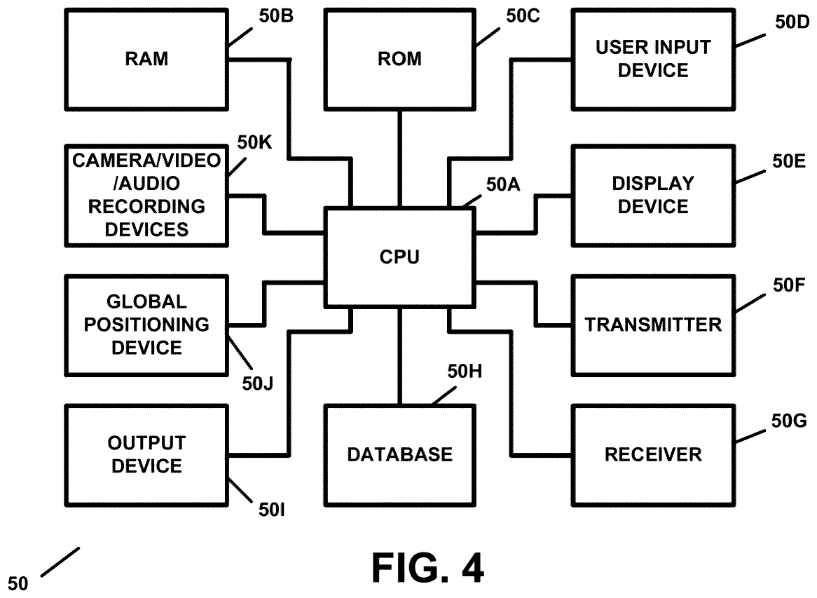

In another preferred embodiment, the apparatus 100 and method of the present invention can be utilized to in order to provide and/or to process real-time payments and/or real-time payment transactions and/or peer-to-peer payments and/or peer-to-peer payment transactions, in and pursuant to healthcare claims payments to healthcare providers and/or other providers. In another preferred embodiment, the apparatus 100 and/or any of the components thereof described and illustrated in FIGS. 1, 2, 3, and 4, herein, can also be a component of, or can be integrated with or within, any apparatus or platform, including any and/or all of those which are disclosed in all of the herein-identified patents and/or patent applications which are incorporated by reference into this application as well as any healthcare platforms, electronic healthcare records and/or any other records and/or telehealth platforms or systems the teachings of which are hereby incorporated by reference herein.

In another preferred embodiment, the apparatus 100 and method of the present invention can be utilized in conjunction with, and/or can be integrated with or within, an electronic health records (EHR) and/or an electronic health records system and/or platform.

In a preferred embodiment, the apparatus 100 and method of the present invention can be used to effectuate a real-time payment or a peer-to-peer payment from a user to a counterparty while providing for the verification of the parties involved and their respective wallet 30 or 60 an/or their respective bank account or financial account.

In a preferred embodiment, the apparatus 100 and method of the present invention can also be utilized so as to ensure, and to verify, before any payment is initiated from a payor's (or user's) wallet or a payor's (or user's) account, that the payment will be effectuated or made to a correct wallet or account of a payee (or counterparty). In another preferred embodiment, the apparatus 100 and method of the present invention can also utilize data and/or information regarding a position or location of the payor (or user) and/or a payee (or counterparty) and/or the respective communication devices 20 or 50 of the payor (or user) or the payee (or counterparty) during a transaction. In another preferred embodiment, the apparatus 100 and method of the present invention can also utilize a picture, a photograph, a video or a video clip, an audio recording or an audio clip, and/or biometric data and/or information, in order to verify the identities of the parties engaging in a real-time payment, a real-time payment transaction, and/or a peer-to-peer payment and/or a peer-to-peer payment transaction.

In a preferred embodiment, the apparatus 100 and method of the present invention can also be utilized to provide an escrow feature, which, in a preferred embodiment, can be effectuated and/or provided by and/or with the central processing computer/distributed ledger technology system 10, and/or by or with the central processing computer 10A and/or the distributed ledger technology system 10B, so as to provide for funds security for and/or during a real-time payment transaction or funds transfer transaction and/or a peer-to-peer payment transaction or funds transfer transaction.

In another preferred embodiment, the apparatus 100 and method of the present invention can also utilize one or any number of smart contracts in order to provide an escrow feature so as to provide for funds security for and/or during a real-time payment transaction or funds transfer transaction and/or a peer-to-peer payment transaction or funds transfer transaction.

In another preferred embodiment, the apparatus 100 and method of the present invention can also be utilized in and/or as a component of, an electronic healthcare record (EHR) system or platform in order to process healthcare insurance payments or healthcare payor payments to a healthcare provider upon the providing of healthcare services or healthcare-related services to an individual or patient.

In another preferred embodiment, the apparatus 100 and method of the present invention can also be utilized in connection with the real-time/peer-to-peer payment provider system 80 or with any one or more, or any number of, real-time/peer-to-peer payment provider system(s) 80, in order to effectuate a real-time payment or a peer-to-peer payment.

In another preferred embodiment, the central processing computer/distributed ledger technology system 10, and/or the central processing computer 10A and/or the distributed ledger technology system 10B, can also be utilized in order to effectuate a real-time payment or a peer-to-peer payment.

FIGS. 5A, 5B, and 5C illustrate a preferred embodiment method for utilizing the apparatus 100 of the present invention, in flow diagram form. In the preferred embodiment of FIGS. 5A, 5B, and 5C, the apparatus 100 of the present invention can be utilized in order to process and/or effectuate a real-time payment and/or a peer-to-peer payment between a user (hereinafter also referred to herein as the “payor”) and a counterparty (hereinafter also referred to herein as the “payee”) in order to ensure that the payment of funds is securely made or effectuated made from the account of the user (the payor) to the account of the counterparty (the payee).

In a preferred embodiment, as well as in any and/or all of the embodiments described herein, it is envisioned that the users and the counterparties will register with, and establish an account with, the apparatus 100, thereby creating respective user accounts and counterparty accounts. In a preferred embodiment, it is envisioned that each user and/or counterparty, who or which utilizes the apparatus 100 and method of the present invention, will register with the apparatus 100 and provide information including, but not limited to, his/her/its name, business name, a unique identifier, such as, but not limited to, an email address, a cellular telephone number, a user identification number, a counterparty identification number, information regarding and/or identifying a respective user communication device 20, user wallet 30, user bank, and/or user bank account number, and/or a respective counterparty communication device 50, counterparty wallet 60, counterparty bank, and/or counterparty bank account number.

In a preferred embodiment, the user or counterparty can also provide a file picture, a file photograph, a file video clip, a file voice clip, a file audio clip, retinal scan data and/or information, fingerprint data and/or information, thumbprint data and/or information, voiceprint data and/or information, and/or any other biometric data and/or information, and/or any other data and/or information, of the respective user or counterparty, which can be used for identify verification and/or for other security related reasons. In a preferred embodiment, the user or counterparty can also provide information regarding accounts, cryptocurrencies, and/or cryptocurrency accounts from which payments can be made or effectuated and/or to which payments can be received. In a preferred embodiment, the user can also provide information regarding restrictions or limitations regarding the use or his/her/its respective account or accounts, transaction amounts, transaction limitations, allowed days and/or times for transactions, allowed goods and/or services for which payments can be made, and/or any other information which can be used for restricting, limiting, allowing, or prohibiting, use of his/her/its respective account or accounts in real-time payments, real-time payment transactions, and/or peer-to-peer payments and/or peer-to-peer payment transactions. In a preferred embodiment, the counterparty can also provide information regarding restrictions or limitations regarding the use or his/her/its respective account or accounts, transaction amounts, transaction limitations, allowed days and/or times for transactions, allowed goods and/or services for which payments can be received, and/or any other information which can be used for restricting, limiting, allowing, or prohibiting, use of his/her/its respective account or accounts in real-time payments, real-time payment transactions, and/or peer-to-peer payments and/or peer-to-peer payment transactions. Any of the foregoing data and/or information can be stored in the database 11H of the central processing computer 10A and can be utilized in any and/or all of the embodiments described herein.

With reference to FIGS. 5A, 5B, and 5C, the operation of the apparatus 100 can commence at step 500. At step 501, in a preferred embodiment, the real-time payment or the peer-to-peer payment can be initiated by the counterparty, as the payee, in a transaction to request and receive a real-time payment or a peer-to-peer payment from a user, individual, or account holder (hereinafter collectively referred to as a “user” or “user payor”).

At step 501, the counterparty (also referred to as the “counterparty payee”) can access the central processing computer 10A with and using the counterparty's counterparty communication device 50. At step 501, the counterparty payee can enter, into the counterparty payee's counterparty communication device 50, information identifying the payment transaction or information for creating a transaction record for the payment transaction, information identifying the user as the payor (“user payor”), user payor identification information, such as, for example, but not limited to, the user payor name, which can be a unique name such as an email address or other unique identifier or unique identifying information regarding the user payor, the amount of the transaction and, as such, the payment amount to be paid to the counterparty payee.

At 501, the counterparty payee can also enter, into the counterparty's counterparty communication device 50, information regarding the reason for the requested payment, information regarding goods or services, which are being, have been, or which are to be, delivered to the user payor, which are the subject of the requested payment, and any other information. At step 501, the counterparty payee can enter, into the counterparty payee's counterparty communication device 50, information regarding and/or identifying the counterparty payee's bank or financial institution and information for identifying the counterparty payee's bank account or financial account to which, or into which, the requested payment is to be made. At step 501, the counterparty payee can enter, into the counterparty payee's counterparty communication device 50, information regarding and/or identifying the counterparty payee's wallet 60 to which, or into which, the requested payment is to be made.

At step 501, the counterparty payee can also transmit from the counterparty payee's counterparty communication device 60, any IP address information and/or location information obtained by the global positioning device 60J. At step 501, the counterparty payee can also transmit from the counterparty payee's counterparty communication device 60 any biometric information, including, but not limited to, data and/or information obtained via a retinal scan, a fingerprint, a thumbprint, and/or any other biometric data and/or measurement of, for, or regarding the counterparty payee.

At step 501, the counterparty payee can also enter, into the counterparty payee's counterparty communication device 50, a picture, photograph, or video clip, or audio clip, of the counterparty payee recorded at and during step 501. Thereafter, at step 501, the counterparty payee's communication device 50 can transmit any and/or all the above-described information, entered into the counterparty payee's communication device 50 at and during step 501, to the central processing computer 10A.

At step 502, the central processing computer 10A can receive and/or store all information transmitted from the counterparty payee's communication device 50 at step 501. In this regard, the central processing computer 10A can receive and store, in the database 11H, any information identifying the payment transaction or information for creating a transaction record for the payment transaction, information identifying the user payor, user payor identification information, such as, for example, but not limited to, the user payor's name, which can be a unique name such as an email address or other unique identifier or unique identifying information regarding the user payor, the amount of the transaction and, as such, the payment amount to be paid to the counterparty payee. At step 502, the central processing computer 10A can also receive and store, in the database 11H, the information regarding the reason for the requested payment, information regarding goods or services, which are being, have been, or which are to be, delivered to the user payor, which are the subject of the requested payment, and any other information. At step 502, the central processing computer 10A can also receive and store, in the database 11H, the information regarding and/or identifying the counterparty payee's bank or financial institution and information for identifying the counterparty payee's bank account or financial account to which, or into which, the requested payment is to be made and/or the information regarding and/or identifying the counterparty payee's wallet 60 to which, or into which, the requested payment is to be made.

At step 502, the central processing computer 10A will process the information received at step 501 and generate a transaction record for the requested payment. At step 502, the central processing computer 10A will process information to verify the counterparty payee's identity and/or to verify that the counterparty payee's bank account or financial account and/or the counterparty payee's wallet 60, identified and entered at step 501, is the same and/or correct bank account or financial account and/or the same and/or correct counterparty payee's wallet 60 which is associated with the counterparty payee which is registered with the apparatus 100.

At step 503, the central processing computer 10A will determine whether or not the counterparty payee's bank account or financial account and/or the counterparty payee's wallet 60, identified and entered at step 501, is verified as being the same and/or the correct bank account or financial account and/or the same and/or correct counterparty payee's wallet 60 which is associated with the counterparty payee which is registered with the apparatus 100. If, at step 503, the central processing computer 10A determines that the counterparty payee's bank account or financial account or that the counterparty payee's wallet 60, identified and entered at step 501, is not the same and/or not the correct bank account or financial account and/or the same and/or correct counterparty payee's wallet 60 registered with the apparatus 100, then requested payment will be declined and cancelled at step 504 and, thereafter, the, at step 505, any data and/or information regarding the attempted request for payment made by the counterparty payee can be stored in the database 11H of the central processing computer 10A. At step 505, any data and/or information regarding the attempted request for payment made by the counterparty payee can also be stored in the distributed ledger technology system 10B. Thereafter, the operation of the apparatus 100 will cease at step 506.

In another preferred embodiment, the central processing computer 10A can, at any of steps 502, 503, 504, 505, and/or 507, provide and/or transmit any information received and/or stored by the central processing computer 10A at any of steps 502, 503, 504, 505, and/or 507 to the distributed ledger technology system 10B and the distributed ledger technology system 10B can use a smart contract or any number of smart contracts in order to perform any and/or all of the processing steps described herein as being performed by the central processing computer 10A at and during steps 502, 503, 504, 505, and/or 507. In this regard any and/or all of the processing routines described herein as being provided by the central processing computer 10A at and during each of steps 502, 503, 504, 505, and/or 507 can be also, and/or instead, be performed by a smart contract or by any number of smart contracts with and using the distributed ledger technology system 10B.

If, however, at step 503, the central processing computer 10A determines that the counterparty payee's bank account or financial account and/or the counterparty payee's wallet 60, identified and entered at step 501, is verified as being the same and/or the correct counterparty payee's bank account or financial account and/or the same and/or correct counterparty payee's wallet 60 which is associated with the counterparty payee which is registered with the apparatus 100, then the operation of the apparatus 100 will proceed to step 507.

At step 507, the central processing computer 10A will process information regarding the requested payment and generate a payment request message. In a preferred embodiment, the payment request message can contain information regarding the identify of the counterparty payee, the amount of the requested payment, the reason for the requested payment, and/or information identifying the user payor and/or any other information which may be deemed helpful for the user payor to identify and/or verify the payment request, the requested payment, or the transaction involving and/or relating to the requested payment. At step 507, the central processing computer 10A will transmit the payment request message to the user payor's user communication device 20.

At step 508, the user payor's user communication device 20 will receive the payment request message and information contained therein can be displayed or otherwise provided to the user payor, or other authorized individual, in visual and/or audio form, via the display device 20E and/or output device 20I. At step 508, the user payor, or other authorized individual for the user payor, can either: 1) confirm that payment will be made; or 2) can decline payment.

At step 508, in the case of a confirmation that the payment will be made, the user payor, or the authorized individual, can enter information into the user payor's user communication device 20 containing information for effectuating, and/or an instruction to effectuate the, payment to the respective bank account or financial account and/or to the counterparty wallet 60 of the counterparty payee, and/or containing information for confirming the transaction and/or the payment request, and/or containing information confirming the transaction amount to be paid, and/or containing information for confirming and/or identifying the user payor's bank account, financial account, cryptocurrency account, or other account, and/or cryptocurrency, and/or combination of the same, from or with which the payment is to me made or effectuated to the counterparty payee's respective bank account, financial account, or the counterparty wallet 60 of the counterparty payee.

At step 508, in the case of the request for payment being declined or denied, the user payor, or the authorized individual, can enter information into the user payor's user communication device 20 in order to decline or deny the payment and/or any information regarding any reason for such declination or denial.

At step 508, the user communication device 20 of the user payor can generate a payment request response message containing any of the foregoing information described herein as being entered into the user communication device 20 of the user payor at step 508.

At step 508, the user payor, or authorized individual, can also transmit from the user payor's user communication device 20, any IP address information and/or location information obtained by the global positioning device 20J. At step 508, the user payor can also transmit from the user payor's user communication device 20 any biometric information, including, but not limited to, data and/or information obtained via a retinal scan, a fingerprint, a thumbprint, and/or any other biometric data and/or measurement of, for, or regarding the counterparty payee. At step 508, the user payor can also enter, into the user payor's user communication device 20, a picture, photograph, or video clip, or audio clip, of the user payor recorded at and during step 508.

Thereafter, at step 508, the user payor's user communication device 20 can transmit any and/or all the above-described information, entered into the user payor's user communication device 20 at and during step 508, and/or the payment request response message to the central processing computer 10A.

At step 509, the central processing computer 10A can receive and/or store the payment request response message and/or any information contained therein. At step 509, the central processing computer 10A can process any of the information contained in the payment request response message. In another preferred embodiment, at step 509, the central processing computer 10A can also process information in order to verify the identity of the user payor and/or for any other purpose.

At step 510, the central processing computer 10A will determine if the payment request has been declined or denied and, if the payment request has been declined or denied, then at step 511, any data and/or information regarding the attempted request for payment made by the counterparty payee can be stored in the database 11H of the central processing computer 10A. At step 511, any data and/or information regarding the attempted request for payment made by the counterparty payee can also be stored in the distributed ledger technology system 10B. At step 511, the central processing computer 10A will also generate a payment request declined message and the central processing computer 10A can, at step 511, transmit the payment request declined message to the counterparty payee's counterparty communication device 50. Thereafter, the operation of the apparatus 100 will cease at step 506.

If, at step 510, the central processing computer 10A determines that the payment request has not been declined or denied, then the operation of the apparatus 100 will proceed to step 512 and the central processing computer 10A will process information contained in the payment request response message in order to verify that the user payor's bank account, financial account, cryptocurrency account, or other account, and/or cryptocurrency, and/or combination of the same, are registered with the user payor.

If at step 512, the central processing computer 10A determines that the user payor's bank account, financial account, cryptocurrency account, or other account, and/or cryptocurrency, and/or combination of the same, identified in the payment request response message is/are not the account(s) or cryptocurrency which is/are registered by or for the user payor, then, at step 513, the payment will be declined or denied, any data and/or information regarding the attempted request for payment made by the user payor can be stored in the database 11H of the central processing computer 10A, and/or can also be stored in the distributed ledger technology system 10B. At step 513, the central processing computer 10A will also generate a payment request declined message and the central processing computer 10A can, at step 514, transmit the payment request declined message to the counterparty payee's counterparty communication device 50. Thereafter, the operation of the apparatus 100 will cease at step 506.

If at step 512, the central processing computer 10A determines that the user payor's bank account, financial account, cryptocurrency account, or other account, and/or cryptocurrency, and/or combination of the same, identified in the payment request response message is/are verified as being the account(s) or cryptocurrency which is/are registered by or for the user payor, then, the operation of the apparatus 100 will proceed to step 514. In a preferred embodiment, the central processing computer 10A, at this point in the operational process at step 514, has verified both the identity of the counterparty payee and the counterparty payee's respective bank account, financial account, and/or counterparty wallet 60, has verified both the identify of the user payor and the user payor's respective bank account, financial account, and/or user wallet 30, and has verified that the payment from the user payor or the counterparty payee is authorized and approved.

At step 514, the central processing computer 10A can process information for instructing and/or effectuating payment from the respective bank account, financial account, or user wallet 20, of the user payor to the respective bank account, financial account, or counterparty wallet 60, of the counterparty payee. At step 514, in a preferred embodiment, the central processing computer 10A can transmit a payment instruction to the real-time/peer-to-peer payment provider system 80 and the real-time/peer-to-peer payment provider system 80, at step 514, can process the necessary information, and/or perform any needed processing routines or tasks in order to transfer funds, and thereby effectuate and/or execute a real-time payment or a peer-to-peer payment from the user payor to the counterparty payee, from the respective bank account or financial account of the user payor to the respective bank account or financial account of the counterparty payee. At step 514, the real-time/peer-to-peer payment provider system 80 will effectuate and/or execute the real-time payment or the peer-to-peer payment from the respective bank account or financial account of the user payor to the respective bank account or financial account of the counterparty payee, and thereby effectuate and/or execute payment to the counterparty payee. At step 514, the real-time/peer-to-peer payment provider system 80 will generate a payment confirmation message and transmit the same to the central processing computer 10A.

At step 515, the central processing computer 10A will receive the payment confirmation message and will store information regarding the same in the database 11H and/or in the distributed ledger technology system 10B. At step 515, the central processing computer 10A can also generate a payment completed message and can transmit the payment completed message to the user communication device 20 of the user payor and to the counterparty communication device 50 of the counterparty payee.

In another preferred embodiment, the central processing computer 10A can, at step 514, process information for effectuating payment of the requested payment and the central processing computer processes the payment and effectuates and/or executes the real-time payment or the peer-to-peer payment from the user wallet 30 of the user payor to the counterparty wallet 60 of the counterparty payee. In this embodiment, wherein the central processing computer 10A processes the payment from the user wallet 30 of the user payor to the counterparty wallet 60 of the counterparty payee, the central processing computer 10A will, at step 515 generate a payment completed message and transmit the payment completed message to the user communication device 20 of the user payor and to the counterparty communication device 50 of the counterparty payee.

In another preferred embodiment, the central processing computer 10A can, at step 514, provide information to, and instruct the distributed ledger technology system 10B to process information for effectuating payment of the requested payment and the distributed ledger technology system 10B processes the payment and effectuates and/or executes the real-time payment or the peer-to-peer payment from the user wallet 30 of the user payor to the counterparty wallet 60 of the counterparty payee. In this embodiment, wherein the distributed ledger technology system 10B processes the payment from the user wallet 30 of the user payor to the counterparty wallet 60 of the counterparty payee, the central processing computer 10A will, upon receiving verification of the completed payment, at step 515 generate a payment completed message and transmit the payment completed message to the user communication device 20 of the user payor and to the counterparty communication device 50 of the counterparty payee.

Once the payment has been effectuated as described herein, the operation of the apparatus 100 will proceed to step 516, and the central processing computer 10A will store any and/or all information regarding the transaction record, and/or any data and/or information, and/or any of the various payment request messages, payment request response messages, payment confirmation messages, payment completed messages, and/or any other messages, described herein, as being transmitted to and/or from the central processing computer 10A from and/or to each of the user communication device 20, the counterparty communication device 60, and/or the real-time/peer-to-peer payment provider system 80, and/or the user wallet 30, and/or the counterparty wallet 60, in the database 11A. The central processing computer 10A will also, at step 516, store any and/or all information regarding the transaction record, and/or any data and/or information, and/or any message, described herein as being transmitted to and/or from the central processing computer 10A from and/or to each of the user communication device 20, the counterparty communication device 60, and/or the real-time/peer-to-peer payment provider system 80, and/or the user wallet 30, and/or the counterparty wallet 60, in the distributed ledger technology system 10B.

Thereafter, the operation of the apparatus 100 will cease at step 506.

In another preferred embodiment, the central processing computer 10A can, at any of steps 508, 509, 510, 511, 512, 513, 514, 515 and/or 516, provide and/or transmit any information received and/or stored by the central processing computer 10A at any of steps 508, 509, 510, 511, 512, 513, 514, 515 and/or 516 to the distributed ledger technology system 10B and the distributed ledger technology system 10B can use a smart contract or any number of smart contracts in order to perform any and/or all of the processing steps described herein as being performed by the central processing computer 10A at and during any of steps 508, 509, 510, 511, 512, 513, 514, 515 and/or 516. In this regard any and/or all of the processing routines described herein as being provided by the central processing computer 10A at and during each of steps 508, 509, 510, 511, 512, 513, 514, 515 and/or 516 can be also, and/or can instead, be performed by a smart contract or by any number of smart contracts with and using the distributed ledger technology system 10B.

In another preferred embodiment, the apparatus 100 can be utilized to process and to effectuate real-time payments and/or peer-to-peer payments involving payments requested by healthcare providers from healthcare insurers and/or healthcare payer pursuant to previously agreed upon payment allowances and/or payment schedules. It is envisioned that healthcare providers and healthcare insurers and/or healthcare payers will agree upon the information submission requirements needed for making claims for payments, and that the healthcare providers and healthcare insurers and/or healthcare payers will agree upon the payment allowances and/or payment schedules for various services rendered to individuals and patients.

In a preferred embodiment, the apparatus 100 and method of the present invention can be integrated into an electronic healthcare record (EHR), and/or any an electronic medical record, an electronic dental record, an electronic pharmacy record, an electronic behavioral health record, or a personal healthcare record, which can, in addition to generating insurance claims forms or messages or request for payments forms or messages, can also provided for the real-time payments and/or the peer-to-peer payments of and for the same.

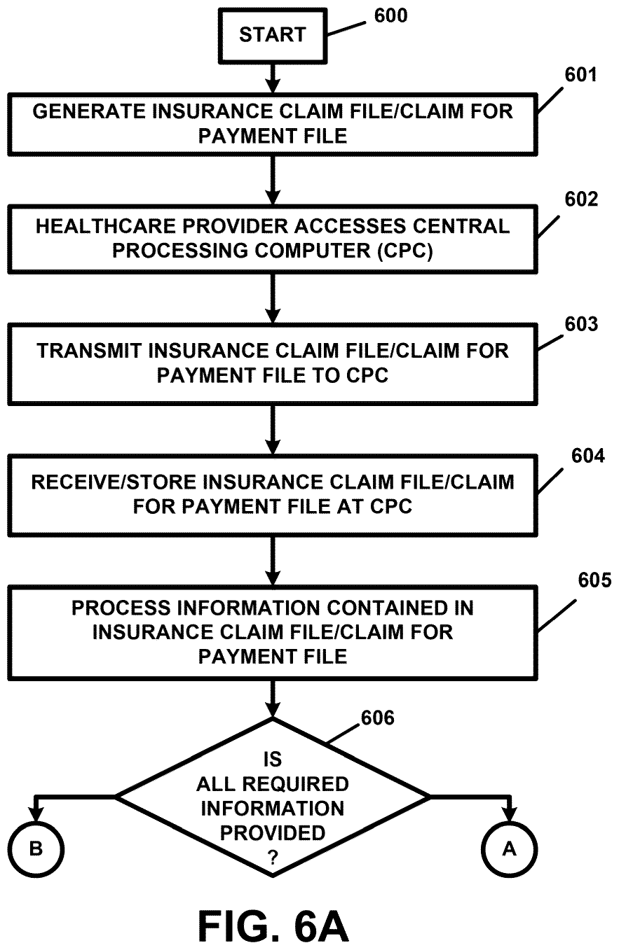

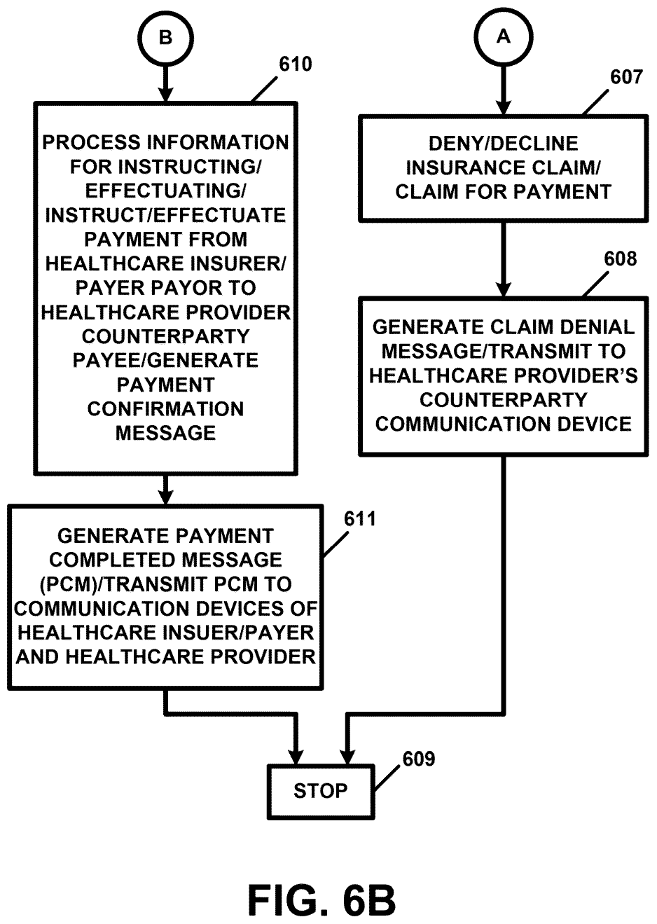

FIGS. 6A and 6B illustrate another preferred embodiment method for utilizing the apparatus and method of the present invention, in block diagram form. With reference to FIGS. 6A and 6B, the apparatus 100 and method of the present invention can be utilized to process and to effectuate real-time payments and/or peer-to-peer payments involving payments requested by healthcare providers from healthcare insurers and/or healthcare payer pursuant to previously agreed upon payment allowances and/or payment schedules, and to do so in a secured manner to ensure proper payments made to and from the correct parties.

With reference once again to FIGS. 6A and 6B, the operation of the apparatus 100 can commence at step 600. In the preferred embodiment of FIGS. 6A and 6B, the real-time payment or the peer-to-peer payment can be initiated by the healthcare provider as the counterparty and payee, when the healthcare provider submits an insurance claim or a claim for payment after providing a service and/or goods or products to and/or for an individual or a patient. In the preferred embodiment, the healthcare insurance company or healthcare payer is the user or payor. In the preferred embodiment, of FIGS. 6A and 6B, the insurance claim or the claim for payment which is generated by the electronic healthcare records systems or the electronic healthcare records platforms which have been incorporated by reference herein via the various U.S. Patent Applications which have been identified as being incorporated by reference herein into this patent application. In a preferred embodiment, the counterparty communication device 60 can be integrated with, and/or can be utilized in conjunction with the electronic healthcare records system, or in conjunction with the electronic healthcare records platform, of a healthcare provider for an individual or patient.

With reference once again the FIGS. 6A and 6B, at step 601, the counterparty communication device 60, using the electronic healthcare records system or the electronic healthcare records platform of the healthcare provider, who or which has provided a healthcare examination, service, diagnosis, or treatment or other service for the individual or patient, will generate an insurance claim file containing information regarding an insurance claim or a claim for payment file containing information regarding a claim for payment.

At step 601, in a preferred embodiment, the insurance claim file or the claim for payment file can contain any and/or all required and/or necessary data and/or information which was previously agreed upon by the healthcare provider and the respective healthcare insurer or healthcare payer as qualifying for automatic payment under the respective insurance claim or claim for payment. In a preferred embodiment, the required and/or necessary data and/or information which was previously agreed upon by the healthcare provider and the respective healthcare insurer or healthcare payer as qualifying for automatic payment under the respective insurance claim or claim for payment can be stored in the database 11A of the central processing computer 10A.

At step 602, the healthcare provider, or authorized user, can access the central processing computer 10A using his/her/its counterparty communication device 50. At step 603, the counterparty communication device 50 can transmit the insurance claim file, containing information regarding the insurance claim, or the claim for payment file, containing information regarding the claim for payment, to the central processing computer 10A. At step 604, the central processing computer 10A will receive and/or store the insurance claim file, containing the information regarding the insurance claim, or the claim for payment file, containing information regarding the claim for payment.

At step 605, the central processing computer will process the information contained in the insurance claim file or the claim for payment file to determine if all of the required and/or necessary data and/or information, which was previously agreed upon by the healthcare provider and the respective healthcare insurer or healthcare payer as qualifying for automatic payment under the respective insurance claim or claim for payment, has been provided in, and/or is contained in, the insurance claim file or the claim for payment file.

If, at step 606, it is determined by the central processing computer 10A that all of the required and/or necessary data and/or information, which was previously agreed upon by the healthcare provider and the respective healthcare insurer or healthcare payer as qualifying for automatic payment under the respective insurance claim or claim for payment, has not been provided in, and/or is not contained in, the insurance claim file or the claim for payment file, then the operation of the apparatus will proceed to step 607, and the central processing computer will decline or deny the insurance claim or the claim for payment. Thereafter, at step 608, the central processing computer 10A will generate a claim denial message and the central processing computer 10A will transmit the claim denial message to the healthcare provider's counterparty communication device 60. Thereafter, the operation of the apparatus 100 will cease at step 609.

If, however, at step 606, it is determined by the central processing computer 10A that all of the required and/or necessary data and/or information, which was previously agreed upon by the healthcare provider and the respective healthcare insurer or healthcare payer as qualifying for automatic payment under the respective insurance claim or claim for payment, has been provided in, and/or is contained in, the insurance claim file or the claim for payment file, then the operation of the apparatus will proceed to step 610.

At step 610, the central processing computer 10A can process information for instructing and/or effectuating payment from the respective bank account, financial account, or user wallet 20, of the healthcare insurer or healthcare payer to the respective bank account, financial account, or counterparty wallet 60, of the healthcare provider.

At step 610, in a preferred embodiment, the central processing computer 10A can transmit a payment instruction to the real-time/peer-to-peer payment provider system 80 and the real-time/peer-to-peer payment provider system 80, at step 610, can process the necessary information, and/or perform any needed processing routines or tasks in order to transfer funds, and thereby effectuate and/or execute a real-time payment or a peer-to-peer payment from the healthcare insurer or healthcare payer to the healthcare provider, from the respective bank account or financial account of the healthcare insurer or healthcare payer to the respective bank account or financial account of the healthcare provider. At step 610, the real-time/peer-to-peer payment provider system 80 will effectuate and/or execute the real-time payment or a peer-to-peer payment from the from the respective bank account or financial account of the healthcare insurer or healthcare payer to the respective bank account or financial account of the healthcare provider, and thereby effectuate payment to the healthcare provider. At step 610, the real-time/peer-to-peer payment provider system 80 will generate a payment confirmation message and transmit the same to the central processing computer 10A.

At step 611, the central processing computer 10A will receive the payment confirmation message and will store information regarding the same in the database 11H and/or in the distributed ledger technology system 10B. At step 611, the central processing computer 10A can also generate a payment completed message and can transmit the payment completed message to the user communication device 20 of the healthcare insurer or healthcare payer and to the counterparty communication device 50 of the healthcare provider.

In another preferred embodiment, the central processing computer 10A can, at step 610, process information for effectuating payment of the requested payment from the user wallet 30 of the healthcare insurer or healthcare payer to the counterparty wallet 60 of the healthcare provider. In this embodiment, wherein the central processing computer 10A processes the payment and effectuates and/or executes the real-time payment or the peer-to-peer payment from the user wallet 30 of the healthcare insurer or healthcare payer to the counterparty wallet 60 of the healthcare provider, the central processing computer 10A will, at step 610 generate a payment completed message and transmit the payment completed message to the user communication device 20 of the healthcare insurer or healthcare payer and to the counterparty communication device 50 of the healthcare provider.

In another preferred embodiment, the central processing computer 10A can, at step 610, provide information to, and instruct the distributed ledger technology system 10B to process information for effectuating payment of the requested payment and the distributed ledger technology system 10B processes the payment and effectuates and/or executes the real-time payment or the peer-to-peer payment from the user wallet 30 of the healthcare insurer or healthcare payer to the counterparty wallet 60 of the healthcare provider. In this embodiment, wherein the distributed ledger technology system 10B processes the payment from the user wallet 30 of the healthcare insurer or healthcare payer to the counterparty wallet 60 of the healthcare provider, the central processing computer 10A will, upon receiving verification of the completed payment, at step 610, generate a payment completed message and transmit the payment completed message to the user communication device 20 of the healthcare insurer or healthcare payer and to the counterparty communication device 50 of the healthcare provider.

Once the payment has been effectuated as described herein, the operation of the apparatus 100 will proceed to step 611, and the central processing computer 10A will store any and/or all information regarding, described herein, as being transmitted to and/or from the central processing computer 10A from and/or to each of the user communication device 20, the counterparty communication device 50, and/or the real-time/peer-to-peer payment provider system 80, and/or the user wallet 30, and/or the counterparty wallet 60, in the database 11A. The central processing computer 10A will also store any and/or all information regarding the insurance claim file or the claim for payment file, and/or any data and/or information contained therein, and/or any data and/or information, and/or any message(s), described herein as being transmitted to and/or from the central processing computer 10A from and/or to each of the user communication device 20, the counterparty communication device 60, and/or the real-time/peer-to-peer payment provider system 80, and/or the user wallet 30, and/or the counterparty wallet 60, in the distributed ledger technology system 10B.

Thereafter, the operation of the apparatus 100 will cease at step 609.

In another preferred embodiment of the embodiment of FIGS. 6A and 6B, the central processing computer 10A can, at any of steps 601, 602, 603, 604, 605, 606, 607, 608, 610, and/or 611, provide and/or transmit any information received and/or stored by the central processing computer 10A at any of steps 601, 602, 603, 604, 605, 606, 607, 608, 610, and/or 611 to the distributed ledger technology system 10B and the distributed ledger technology system 10B can use a smart contract or any number of smart contracts in order to perform any and/or all of the processing steps described herein as being performed by the central processing computer 10A at and during any of steps any of steps 601, 602, 603, 604, 605, 606, 607, 608, 610, and/or 611. In this regard any and/or all of the processing routines described herein as being provided by the central processing computer 10A at and during each of steps any of steps 601, 602, 603, 604, 605, 606, 607, 608, 610, and/or 611 can be also, and/or can instead, be performed by a smart contract or by any number of smart contracts with and using the distributed ledger technology system 10B.

In another preferred embodiment of the embodiments of FIGS. 5A, 5B, and 5C and FIGS. 6A and 6B, the apparatus 100 of the present invention also be utilized and can work in conjunction with, can be integrated into or with, and/or can perform all of the functions and functionalities disclosed and described as being performed by each of, the respective apparatuses and their various and respective central processing computers, computers, and/or communication devices, which are disclosed and described in, for example, U.S. patent application Ser. No. 17/224,838, filed Apr. 7, 2021, and entitled “APPARATUS AND METHOD FOR PROVIDING HEALTHCARE SERVICES REMOTELY OR VIRTUALLY WITH OR USING AN ELECTRONIC HEALTHCARE RECORD AND/OR A COMMUNICATION NETWORK”, U.S. Provisional Patent Application Ser. No. 63/634,841, filed Apr. 16, 2024, and entitled “APPARATUS AND METHOD FOR PROVIDING HEALTHCARE SERVICES REMOTELY OR VIRTUALLY WITH OR USING AN ELECTRONIC HEALTHCARE RECORD AND/OR A COMMUNICATION NETWORK AND/OR WITH OR USING A PERSONAL MONITORING DEVICE”, filed April 2024, and U.S. Provisional Patent Application Ser. No. 63/643,488, filed May 7, 2025, and entitled “APPARATUS AND METHOD FOR PROVIDING HEALTHCARE SERVICES REMOTELY OR VIRTUALLY WITH OR USING AN ELECTRONIC HEALTHCARE RECORD AND/OR A COMMUNICATION NETWORK AND/OR WITH OR USING A PERSONAL MONITORING DEVICE”, the subject matter and teachings of all of which are hereby incorporated by reference herein in their entirety, as well as described and disclosed in each of all of the other U.S. Patent Applications and U.S. provisional Patent Applications the subject matter and teachings of which are also incorporated by reference herein.

In another preferred embodiment, the embodiment of FIGS. 5A, 5B, and 5C can also be utilized by healthcare providers to request automatic payment upon automated approval of a healthcare insurance claim or a claim for payment from a healthcare insurer of a healthcare payer.

In another preferred embodiment, as well as in any and/or all of the preferred embodiments described herein, the central processing computer 10A can process IP address information or position or location information for a respective user communication device 20 and/or a counterparty communication device 50 in or order to verify an identify and/or an authority of a respective user payor and/or a counterparty payee at and during any appropriate point or step in the embodiments of FIGS. 5A, 5B, and 5C, in the embodiments of FIGS. 6A and 6B, and/or at any other time and/or for any other purpose.

In another preferred embodiment, as well as in any and/or all of the preferred embodiments described herein, a smart contract or any number of smart contracts utilized in the distributed ledger technology system 10B can process IP address information or position or location information for a respective user communication device 20 and/or a counterparty communication device 50 in or order to verify an identify and/or an authority of a respective user payor and/or a counterparty payee at and during any appropriate point or step in the embodiments of FIGS. 5A, 5B, and 5C, in the embodiments of FIGS. 6A and 6B, and/or at any other time and/or for any other purpose.

In another preferred embodiment, as well as in any and/or all of the preferred embodiments described herein, the central processing computer 10A can process information obtained from a fingerprint recognition device, a voice recognition device, a retinal scanner, a fingerprint device, a thumbprint device, a voice recognition device, a handprint recognition device, a handprint geometry recognition device, a facial feature recognition device, and/or any one or more of the biometric devices, in or order to verify an identify and/or an authority of a respective user payor and/or a counterparty payee at and during any appropriate point or step in the embodiments of FIGS. 5A, 5B, and 5C, in the embodiments of FIGS. 6A and 6B, and/or at any other time and/or for any other purpose.

In another preferred embodiment, as well as in any and/or all of the preferred embodiments described herein, a smart contract or any number of smart contracts utilized in the distributed ledger technology system 10B can process information obtained from a fingerprint recognition device, a voice recognition device, a retinal scanner, a fingerprint device, a thumbprint device, a voice recognition device, a handprint recognition device, a handprint geometry recognition device, a facial feature recognition device, and/or any one or more of the biometric devices, in or order to verify an identify and/or an authority of a respective user payor and/or a counterparty payee at and during any appropriate point or step in the embodiments of FIGS. 5A, 5B, and 5C, in the embodiments of FIGS. 6A and 6B, and/or at any other time and/or for any other purpose.

In another preferred embodiment, as well as in any and/or all of the preferred embodiments described herein, the central processing computer 10A can process information for verifying a bank account, financial account, or other account or cryptocurrency, and/or respective user wallet 30 or counterparty wallet 60 in or order to verify an identify or authority of a respective user payor and/or a counterparty payee at and during any appropriate point or step in the embodiments of FIGS. 5A, 5B, and 5C, in the embodiments of FIGS. 6A and 6B, and/or at any other time and/or for any other purpose.

In another preferred embodiment, as well as in any and/or all of the preferred embodiments described herein, a smart contract or any number of smart contracts utilized in the distributed ledger technology system 10B can process information for verifying a bank account, financial account, or other account or cryptocurrency, and/or respective user wallet 30 or counterparty wallet 60 in or order to verify an identify or authority of a respective user payor and/or a counterparty payee at and during any appropriate point or step in the embodiments of FIGS. 5A, 5B, and 5C, in the embodiments of FIGS. 6A and 6B, and/or at any other time and/or for any other purpose.

In another preferred embodiment, as well as in any and/or all of the preferred embodiments described herein, the central processing computer 10A can process information regarding an limitation(s) and/or restriction(s) placed by a user payor on or involving any bank account, financial account, and/or other account, and/or any cryptocurrency, in order to determine if a real-time payment or a peer-to-peer payment is allowed or authorized.

In another preferred embodiment, as well as in any and/or all of the preferred embodiments described herein, a smart contract or any number of smart contracts utilized in the distributed ledger technology system 10B can process information regarding an limitation(s) and/or restriction(s) placed by a user payor on or involving any bank account, financial account, and/or other account, and/or any cryptocurrency, in order to determine if a real-time payment or a peer-to-peer payment is allowed or authorized.

In another preferred embodiment of the embodiments of each of FIGS. 5A and 5B and 6A and 6B, as well as in any and/or all of the preferred embodiments described herein, the central processing computer 10A can process information regarding an limitation(s) and/or restriction(s) placed by a user payor on or involving any bank account, financial account, and/or other account, and/or any cryptocurrency, in order to determine if a real-time payment or a peer-to-peer payment is allowed or authorized.

In another preferred embodiment, as well as in any and/or all of the preferred embodiments described herein, a smart contract or any number of smart contracts utilized in the distributed ledger technology system 10B can process information regarding an limitation(s) and/or restriction(s) placed by a user payor on or involving any bank account, financial account, and/or other account, and/or any cryptocurrency, in order to determine if a real-time payment or a peer-to-peer payment is allowed or authorized.

In another preferred embodiment of the embodiments of each of FIGS. 5A and 5B and 6A and 6B, as well as in any and/or all of the preferred embodiments described herein, a smart contract or any number of smart contracts utilized in the distributed ledger technology system 10B can process information regarding an limitation(s) and/or restriction(s) placed by a user payor on or involving any bank account, financial account, and/or other account, and/or any cryptocurrency, in order to determine if a real-time payment or a peer-to-peer payment is allowed or authorized

In another preferred embodiment, as well as in any and/or all of the preferred embodiments described herein, the central processing computer 10A can process information for instructing, effectuating, and/or providing notification to a user payor and/or a counterparty payee, a real-time payment or a peer-to-peer payment.

In another preferred embodiment of the embodiments of each of FIGS. 5A and 5B and 6A and 6B, as well as in any and/or all of the preferred embodiments described herein, the central processing computer 10A can process information for instructing, effectuating, and/or providing notification to a user payor and/or a counterparty payee, a real-time payment or a peer-to-peer payment.

In another preferred embodiment, as well as in any and/or all of the preferred embodiments described herein, a smart contract or any number of smart contracts utilized in the distributed ledger technology system 10B can process information for instructing, effectuating, and/or providing notification to a user payor and/or a counterparty payee, a real-time payment or a peer-to-peer payment.

In another preferred embodiment of the embodiments of each of FIGS. 5A and 5B and 6A and 6B, as well as in any and/or all of the preferred embodiments described herein, a smart contract or any number of smart contracts utilized in the distributed ledger technology system 10B can process information for instructing, effectuating, and/or providing notification to a user payor and/or a counterparty payee, a real-time payment or a peer-to-peer payment.

In another preferred embodiment, the apparatus 100 and method of the present invention can be utilized in conjunction with, and/or can be integrated with or within, any apparatus, system, or platform, which can involve any processing of any real-time payment or peer-to-peer payment.

The apparatus 100 and method of the present invention provides for substantial technological improvements to and for real-time payment systems computers and/or platforms, and/or peer-to-peer payment systems computers and/or platforms. The apparatus 100 and method of the present invention also provides for substantial technological improvements to and for electronic healthcare records computers, systems, and/or platforms and/or telehealth computers, systems, and/or platforms, which can provide for the processing of real-time payments and/or peer-to-peer payments, via the same.

The apparatus 100 and method of the present invention provides for substantial technological improvements to and for real-time payment systems computers and/or platforms, and/or peer-to-peer payment systems computers and/or platforms which can provide enhanced security for and involving real-time payments and/or peer-to-peer payments. The apparatus 100 and method of the present invention also provides for substantial technological improvements to and for electronic healthcare records computers, systems, and/or platforms and/or telehealth computers, systems, and/or platforms, which can provide enhanced security for and involving real-time payments and/or peer-to-peer payments.

While the present invention has been described and illustrated in various preferred and alternate embodiments, such descriptions are merely illustrative of the present invention and are not to be construed to be limitations thereof. In this regard, the present invention encompasses all modifications, variations and/or alternate embodiments, with the scope of the present invention being limited only by the claims which follow.

What is claimed is:

1. An apparatus, comprising:
a central processing computer;
a distributed ledger technology system; and
a payment provider system, wherein the payment provider system is configured to process information for, and to execute, a real-time payment or a peer-to-peer payment from a user payor account, associated with a user payor, to a counterparty payee account, associated with a counterparty payee,
wherein the central processing computer receives information regarding a requested payment, wherein the information regarding the requested payment is transmitted from a counterparty communication device associated with the counterparty payee, wherein the central processing computer processes information regarding the requested payment and verifies the counterparty payee account, and further wherein the central processing computer generates a payment request message, and further wherein the central processing computer transmits the payment request message to a user communication device associated with the user payor, and further wherein the central processing computer receives a payment request response message transmitted from the user communication device,
wherein the central processing computer verifies the user payor account, and further wherein the central processing computer processes information for instructing, and instructs, the payment provider system to execute a real-time payment or a peer-to-peer payment from the user payor account to the counterparty payee account, or wherein the central processing computer processes information and executes a real-time payment or a peer-to-peer payment from the user payor account to the counterparty payee account, or wherein the central processing computer processes information for instructing, and instructs, the distributed ledger technology system to execute a real-time payment or a peer-to-peer payment from the user payor account to the counterparty payee account,
and further wherein, upon execution of, or upon confirmation of, the occurrence of the real-time payment or the peer-to-peer payment from the user payor account to the counterparty payee account, the central processing computer generates a payment completed message and transmits the payment completed message to the user communication device and to the counterparty communication device, and further wherein the central processing computer stores information regarding the real-time payment or the peer-to-peer payment from the user payor account to the counterparty payee account in a database, in the distributed ledger technology system, or in a database and the distributed ledger technology system.

2. The apparatus of claim 1, wherein the payment provider system executes the real-time payment or the peer-to-peer payment from the user payor account to the counterparty payee account.

3. The apparatus of claim 1, wherein the central processing computer executes the real-time payment or the peer-to-peer payment from the user payor account to the counterparty payee account.

4. The apparatus of claim 1, wherein the distributed ledger technology system executes the real-time payment or the peer-to-peer payment from the user payor account to the counterparty payee account.

5. The apparatus of claim 4, wherein the distributed ledger technology system executes the real-time payment or the peer-to-peer payment from the user payor account to the counterparty payee account utilizing a smart contract or a plurality of smart contracts, or wherein a smart contract executes or a plurality of smart contracts execute the real-time payment or the peer-to-peer payment from the user payor account to the counterparty payee account.

6. The apparatus of claim 1, wherein the central processing computer or the distributed ledger technology system processes information for verifying the counterparty payee account using IP address information or position or location information regarding the counterparty communication device.

7. The apparatus of claim 1, wherein the central processing computer or the distributed ledger technology system processes information for verifying the user payor account using IP address information or position or location information regarding the user communication device.

8. The apparatus of claim 1, wherein the central processing computer or the distributed ledger technology system processes information regarding the requested payment using information regarding a restriction or a limitation regarding a use of the user payor account.

9. The apparatus of claim 1, wherein the central processing computer or the distributed ledger technology system processes information for verifying the user payor account using biometric information regarding the user payor, or wherein the central processing computer or the distributed ledger technology system processes information for verifying the counterparty payee account using biometric information regarding the counterparty payee.

10. The apparatus of claim 1, wherein the apparatus effectuates the real-time payment or the peer-to-peer payment from the user payor account via a user wallet.

11. The apparatus of claim 1, wherein the information regarding a requested payment is, or in contained in, a healthcare insurance claim or a claim for payment.

12. The apparatus of claim 11, wherein the central processing computer or the distributed ledger technology system processes information regarding the healthcare insurance claim or the claim for payment and verifies that all required information is included in the healthcare insurance claim or the claim for payment.

13. The apparatus of claim 1, wherein the apparatus, the central processing computer, or the distributed ledger technology system, is, or is a component of, or is integrated with or within, an electronic healthcare record (EHR), an electronic healthcare records system, an electronic healthcare records computer, or an electronic healthcare records platform, or is, or is a component of, or is integrated with or within, a telehealth system, a telehealth computer, or a telehealth platform.

14. The apparatus of claim 1, wherein the payment provider system is, or is a component of, or is integrated with or within, an electronic healthcare record (EHR), an electronic healthcare records system, an electronic healthcare records computer, or an electronic healthcare records platform, or is, or is a component of, or is integrated with or within, a telehealth system, a telehealth computer, or a telehealth platform.

15. An apparatus, comprising:
a central processing computer;
a distributed ledger technology system; and
a payment provider system, wherein the payment provider system is configured to process information for, and to execute, pursuant to a healthcare insurance claim or a claim for payment, a real-time payment or a peer-to-peer payment from a payor account, associated with a healthcare insurer or a healthcare payer, to a counterparty payee account, associated with a healthcare provider,
wherein the central processing computer receives information regarding a healthcare insurance claim or a claim for payment, wherein the information regarding the healthcare insurance claim or the claim for payment is transmitted from a counterparty communication device associated with the healthcare provider, wherein the central processing computer processes information regarding the healthcare insurance claim or the claim for payment and verifies that all required information is included in the healthcare insurance claim or the claim for payment for a payment to be made, and further wherein the central processing computer processes information for instructing, and instructs, the payment provider system to execute a real-time payment or a peer-to-peer payment from the payor account associated with the healthcare insurer or the healthcare payer, to the counterparty payee account associated with the healthcare provider, or wherein the central processing computer processes information and executes a real-time payment or a peer-to-peer payment from the payor account associated with the healthcare insurer or the healthcare payer, to the counterparty payee account associated with the healthcare provider, or wherein the central processing computer processes information for instructing, and instructs, the distributed ledger technology system to execute a real-time payment or a peer-to-peer payment from the payor account associated with the healthcare insurer or the healthcare payer, to the counterparty payee account associated with the healthcare provider,
and further wherein, upon execution of, or upon confirmation of, the occurrence of the real-time payment or the peer-to-peer payment from the payor account associated with the healthcare insurer or the healthcare payer, to the counterparty payee account associated with the healthcare provider, the central processing computer generates a payment completed message and transmits the payment completed message to the user communication device and to the counterparty communication device, and further wherein the central processing computer stores information regarding the real-time payment or the peer-to-peer payment from the user payor account to the counterparty payee account in a database, in the distributed ledger technology system, or in a database and the distributed ledger technology system.

16. The apparatus of claim 15, wherein the apparatus, the central processing computer, or the distributed ledger technology system, is, or is a component of, or is integrated with or within, an electronic healthcare record (EHR), an electronic healthcare records system, an electronic healthcare records computer, or an electronic healthcare records platform, or is, or is a component of, or is integrated with or within, a telehealth system, a telehealth computer, or a telehealth platform.

17. The apparatus of claim 15, wherein the payment provider system is, or is a component of, or is integrated with or within, an electronic healthcare record (EHR), an electronic healthcare records system, an electronic healthcare records computer, or an electronic healthcare records platform, or is, or is a component of, or is integrated with or within, a telehealth system, a telehealth computer, or a telehealth platform.

18. The apparatus of claim 15, wherein the payment provider system executes the real-time payment or the peer-to-peer payment from the payor account to the counterparty payee account.

19. The apparatus of claim 15, wherein the central processing computer executes the real-time payment or the peer-to-peer payment from the payor account to the counterparty payee account.

20. The apparatus of claim 15, wherein the distributed ledger technology system executes the real-time payment or the peer-to-peer payment from the payor account to the counterparty payee account.
